(19) United States
(12) Patent Application Publication
(10) Pub. No.: US 2022/0298160 A1
(43) Pub. Date: Sep. 22, 2022

(54) COMPOUNDS USEFUL IN HIV THERAPY

(71) Applicants: GLAXOSMITHKLINE INTELLECTUAL PROPERTY (NO.2) LIMITED, Brentford, Middlesex (GB); VIIV HEALTHCARE COMPANY, Wilmington, DE (US)

(72) Inventors: Martha Alicia DE LA ROSA, Research Triangle Park, NC (US); John MILLER, Research Triangle Park, NC (US); Lita S. SUWANDI, Research Triangle Park, NC (US); David TEMELKOFF, Research Triangle Park, NC (US); Emile Johann VELTHUISEN, Collegeville, NC (US)

(73) Assignees: GLAXOSMITHKLINE INTELLECTUAL PROPERTY (NO.2) LIMITED, Brentford, Middlesex (GB); VIIV HEALTHCARE COMPANY, Wilmington, DE (US)

(21) Appl. No.: 17/635,776

(22) PCT Filed: Aug. 28, 2020
(86) PCT No.: PCT/IB2020/058046

§ 371 Date: Feb. 16, 2022

Related U.S. Application Data
(60) Provisional application No. 62/892,577, filed on Aug. 28, 2019.

(51) Int. Cl. C07D 473/34

(52) U.S. Cl. CPC C07D 473/34

(57) ABSTRACT

The invention relates to compounds of Formulae (I)-(II), salts thereof, pharmaceutical compositions thereof, as well as methods of treating or preventing HIV in subjects.

### FIELD OF THE INVENTION

The present invention relates to compounds, pharmaceutical compositions, and methods of use thereof in connection with individuals infected with HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/S235230181630087X?via%3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Hutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/guidelines

Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example, an individual on a regimen containing 3TC/FTC may select for the M184V mutation that reduces susceptibility to these drugs by >100 fold. See e g., https://hivdb.stanford.edu/dr-summary/resistance-notes/NRTI

Another way to potentially address preventing formation of mutations is to increase patient adherence to a drug regimen. One manner that may accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may the shortcomings set forth above.

### SUMMARY OF THE INVENTION

In one aspect, the invention provides a compound of the formula (I):

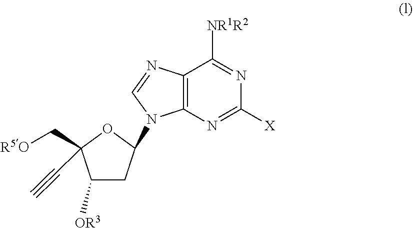

wherein:
X is selected from the group consisting of NH₂, F and Cl
R¹ and R² are each independently selected from the group consisting of H,

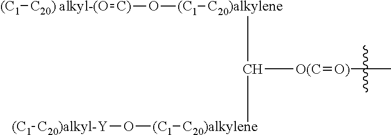

wherein Y is a bond or —C(═O)—
; and

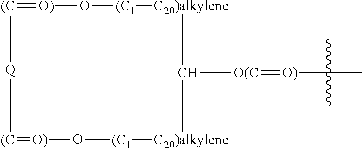

wherein Q is (C₃-C₁₅)alkylene;

R³ is selected from the group consisting of H and —C(═O)—Z—R⁴, wherein Z is a bond or —O—, and wherein R⁴ is selected from the group consisting of:

(1) NR⁵R⁶ wherein R⁵ is H or (C₁-C₆)alkyl and R⁶ is selected from the group consisting of (C₁-C₂₀)alkyl, (C₃-C₆)cycloalkyl; and —(CH₂)ₘ—(C═O)—O—(C₁-C₂₀)alkyl wherein m is an integer ranging from 1 to 10; or wherein R⁵ and R⁶ join to form a (C₃-C₆)heterocycle;

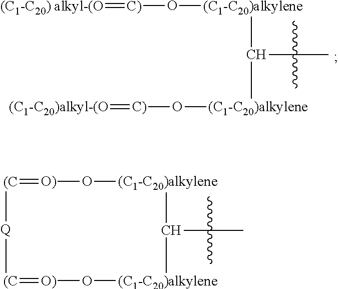

wherein Q is (C₃-C₁₅)alkylene; and

(4) —(C₁-C₆)alkylene-aryl-R¹¹, wherein R¹¹ is selected from H and —O(C═O)—(C₁-C₂₀)alkyl; and R^{5′} is selected from the group consisting of H,

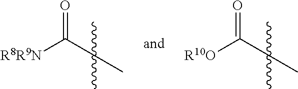

wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₀)alkyl;

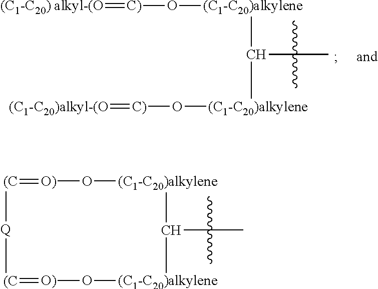

wherein Q is (C₃-C₁₅)alkylene;

wherein when R³ and R^{5′} are each H and X is F or Cl, both R¹ and R² cannot be H;

or a pharmaceutically acceptable salt thereof.

In another aspect, the invention provides a compound of the formula (II):

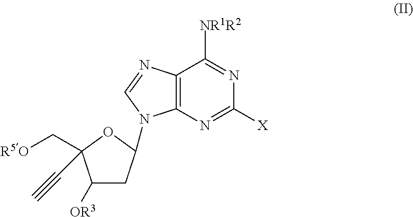

wherein:
X is selected from the group consisting of NH₂, F and Cl;
R¹ and R² are each independently selected from the group consisting of H,

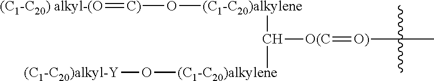

wherein Y is a bond or —C(═O)—
; and

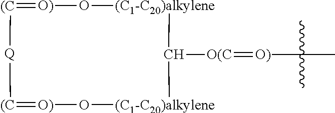

wherein Q is (C₃-C₁₅)alkylene;

R³ is selected from the group consisting of H and —C(═O)—Z—R⁴, wherein Z is a bond or —O—, and wherein R⁴ is selected from the group consisting of:

(1) NR⁵R⁶ wherein R⁵ is H or (C₁-C₆)alkyl and R⁶ is selected from the group consisting of (C₁-C₂₀)alkyl, (C₃-C₆)cycloalkyl; and —(CH₂)ₘ—(C═O)—O—(C₁-C₂₀)alkyl wherein m is an integer ranging from 1 to 10; or wherein R⁵ and R⁶ join to form a (C₃-C₆)heterocycle;

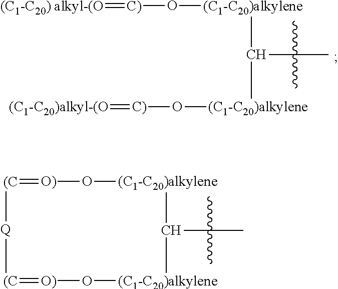

wherein Q is (C₃-C₁₅)alkylene; and

(4) —(C₁-C₆)alkylene-aryl-R¹¹, wherein R¹¹ is selected from H and —O(C═O)—(C₁-C₂₀)alkyl; and R^{5′} is selected from the group consisting of H,

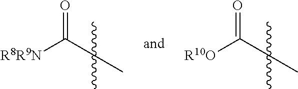

wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₀)alkyl;

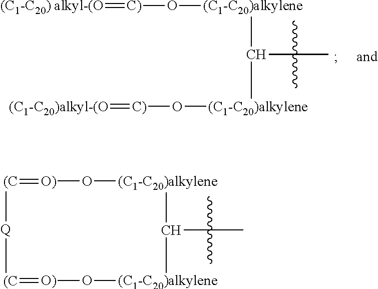

wherein Q is (C₃-C₁₅)alkylene;

wherein when R³ and R^{5′} are each H and X is F or Cl, both R¹ and R² cannot be H;

or a pharmaceutically acceptable salt thereof.

In another aspect, the invention provides pharmaceutical compositions comprising a compound of Formulas (I)-(II) a pharmaceutically acceptable salt thereof and an excipient

In another aspect, the invention provides a method of treating or preventing an HIV infection in a subject at risk for developing an HIV infection, comprising administering to the subject a compound of Formulas (I-(II), or a pharmaceutically acceptable salt thereof.

In another aspect, there is provided a compound of Formulas (I)-(II) or a pharmaceutically acceptable salt thereof for use in therapy.

In another aspect, there is provided a compound of Formulas (I)-(II) or a pharmaceutically acceptable salt thereof for use in treating or preventing an HIV infection.

In another aspect, there is provided the use of a compound of Formulas (I)-(II) or a pharmaceutically acceptable salt thereof in the manufacture of a medicament for treating or preventing an HIV infection. These and other aspects are encompassed by the invention as set forth herein.

### BRIEF DESCRIPTION OF THE DRAWINGS

FIG. 1 illustrates the mean whole blood concentration-time profiles of Compound 9 and EFdA after single intramuscular injection of Compound 9 at 20 mg/kg in male Wistar Han rats (N=3/time point); and

FIG. 2 illustrates the mean whole blood concentration-time profiles of Compound 30 and EFdA after single intramuscular injection of Compound 30 at 20 mg/kg in male Wistar Han rats (N=3/time point)

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

Where used herein the terms such as “a compound of formula (I)-(II)” and “compounds of formulae (I)-(II)” are intended to refer to each and all of the compounds defined herein, i.e., the compounds of formulae (I) and (II).

“Alkyl” refers to a monovalent saturated aliphatic hydrocarbon group having from, e.g., 1 to 25 carbon, e.g., 1 to 10 carbon atoms atoms and, in some embodiments, from 1 to 6 carbon atoms. “(Cₓ-C_{y}) alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). For the purposes of the invention, alkyl may encompass alkylene groups as defined below.

“Alkylene” refers to divalent saturated aliphatic hydrocarbon groups that may having e.g., from 1 to 25 carbon atoms. The alkylene groups include branched and straight chain hydrocarbyl groups. For example, “(C₁-C₆)alkylene” is meant to include methylene, ethylene, propylene, 2-methypropylene, dimethylethylene, pentylene, and so forth. As such, the term “propylene” could be exemplified by the following structure:

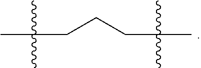 [SMILES: *CCC*]

Likewise, the term “dimethylbutylene” could be exemplified by any of the following three structures or more:

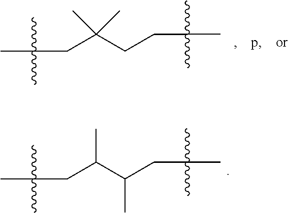

Furthermore, the term “(C₁-C₆)alkylene” is meant to include such branched chain hydrocarbyl groups as cyclopropylmethylene, which could be exemplified by the following structure:

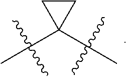 [SMILES: *C1(*)CC1]

“Alkenyl” refers to a linear or branched hydrocarbon group having, e.g., from 2 to 25, e.g., 2 to 20, e.g., 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropylene, 1,3-butadienyl, and the like Polyalkenyl substituents are also encompassed by this definition.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₂₅), (C₂-C₂₀), or (C₂-C₆)alkynyl is meant to include ethynyl, propynyl, and the like. Polyalkynyl substituents are also encompassed by this definition.

“AUC” refers to the area under the plot of plasma concentration of drug (not logarithm of the concentration) against time after drug administration.

“EC₅₀” refers to the concentration of a drug that gives half-maximal response.

“IC₅₀” refers to the half-maximal inhibitory concentration of a drug. Sometimes, it is also converted to the pIC₅₀ scale (−log IC₅₀), in which higher values indicate exponentially greater potency.

“Haloalkyl” refers to substitution of an alkyl group with 1 to 3 halo groups (e.g., bifluoromethyl or trifluoromethyl).

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

“Polymorphism” refers to when two or more clearly different phenotypes exist in the same population of a species where the occurrence of more than one form or morph. In order to be classified as such, morphs must occupy the same habitat at the same time and belong to a panmictic population (one with random mating).

“Racemates” refers to a mixture of enantiomers. In an embodiment of the invention, the compounds of Formulas I or II or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N-moiety such as pyrazoles, imidazoles, benzimidazoles, triazoles, and tetrazoles.

The term ‘atropisomer’ refers to a stereoisomer resulting from an axis of asymmetry. This can result from restricted rotation about a single bond where the rotational barrier is high enough to allow differentiation of the isomeric species up to and including complete isolation of stable non-interconverting diastereomer or enantiomeric species. One skilled in the art will recognize that upon installing a nonsymmetrical R^{x} to core, the formation of atropisomers is possible. In addition, once a second chiral center is installed in a given molecule containing an atropisomer, the two chiral elements taken together can create diastereomeric and enantiomeric stereochemical species. Depending upon the substitution about the Cx axis, interconversion between the atropisomers may or may not be possible and may depend on temperature. In some instances, the atropisomers may interconvert rapidly at room temperature and not resolve under ambient conditions. Other situations may allow for resolution and isolation but interconversion can occur over a period of seconds to hours or even days or months such that optical purity is degraded measurably over time. Yet other species may be completely restricted from interconversion under ambient and/or elevated temperatures such that resolution and isolation is possible and yields stable species. When known, the resolved atropisomers were named using the helical nomenclature. For this designation, only the two ligands of highest priority in front and behind the axis are considered. When the turn priority from the front ligand 1 to the rear ligand 1 is clockwise, the configuration is P, if counterclockwise it is M.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” or “subject” refers to mammals and includes humans and non-human mammals.

Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Where specific compounds or generic formulas are drawn that have aromatic rings, such as aryl or heteroaryl rings, then it will be understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

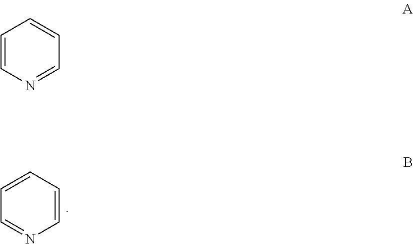 [SMILES: c1ccncc1 ; c1ccncc1]

The present invention includes compounds as well as their pharmaceutically acceptable salts. Accordingly, the word “or” in the context of “a compound or a pharmaceutically acceptable salt thereof” is understood to refer to either: 1) a compound alone or a compound and a pharmaceutically acceptable salt thereof (alternative), or 2) a compound and a pharmaceutically acceptable salt thereof (in combination).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “—C(R^{x})₂”, it should be understood that the two R^{x} groups can be the same, or they can be different if R^{x} is defined as having more than one possible identity. In addition, certain substituents are drawn as —R^{x}R^{y}, where the “—” indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

In one aspect, the invention provides a compound of the formula (I):

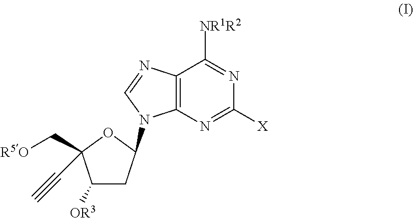

wherein:
X is selected from the group consisting of NH₂, F and Cl;
R¹ and R² are each independently selected from the group consisting of H,

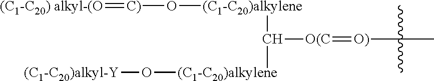

wherein Y is a bond or —C(═O)—
; and

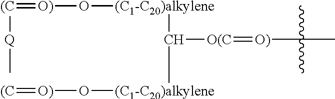

wherein Q is (C₃-C₁₅)alkylene;

R³ is selected from the group consisting of H and —C(═O)—Z—R⁴, wherein Z is a bond or —O—, and wherein R⁴ is selected from the group consisting of:

(1) NR⁵R⁶ wherein R⁵ is H or (C₁-C₆)alkyl and R⁶ is selected from the group consisting of (C₁-C₂₀)alkyl, (C₃-C₆)cycloalkyl; and —(CH₂)ₘ—(C═O)—O—(C₁-C₂₀)alkyl wherein m is an integer ranging from 1 to 10; or wherein R⁵ and R⁶ join to form a (C₃-C₆)heterocycle;

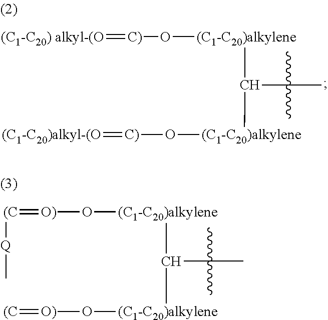

wherein Q is (C₃-C₁₅)alkylene; and

(4) —(C₁-C₆)alkylene-aryl-R¹¹, wherein R¹¹ is selected from H and —O(C═O)—(C₁-C₂₀)alkyl; and R^{5′} is selected from the group consisting of H,

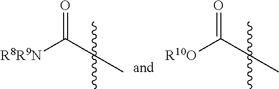

wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₅)alkyl;

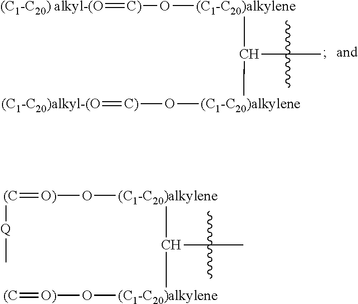

wherein Q is (C₃-C₁₅)alkylene;

wherein when R³ and R^{5′} are each H and X is F or Cl, both R¹ and R² cannot be H; or a pharmaceutically acceptable salt thereof.

In another aspect, the invention provides a compound of the formula (II):

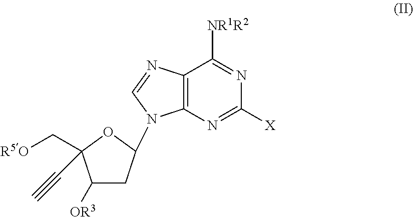

wherein:
X is selected from the group consisting of NH₂, F and Cl;
R¹ and R² are each independently selected from the group consisting of H,

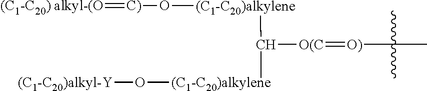

wherein Y is a bond or —C(═O)—
; and

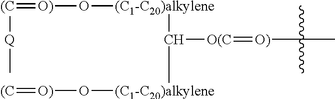

wherein Q is (C₃-C₁₅)alkylene;

R³ is selected from the group consisting of H and —C(═O)—Z—R⁴, wherein Z is a bond or —O—, and wherein R⁴ is selected from the group consisting of:

(1) NR⁵R⁶ wherein R⁵ is H or (C₁-C₆)alkyl and R⁶ is selected from the group consisting of (C₁-C₂₀)alkyl, (C₃-C₆)cycloalkyl; —(CH₂)ₘ—(C═O)—O—(C₁-C₂₀)alkyl wherein m is an integer ranging from 1 to 10; or wherein R⁵ and R⁶ join to form a (C₃-C₆)heterocycle;

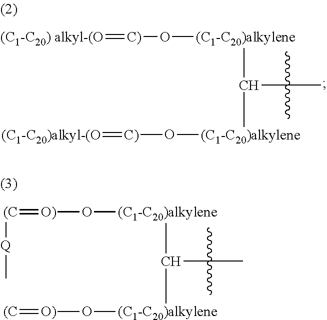

wherein Q is (C₃-C₁₅)alkylene; and

(4) —(C₁-C₆)alkylene-aryl-R¹¹, wherein R¹¹ is selected from H and —O(C═O)—(C₁-C₂₀)alkyl; and R^{5′} is selected from the group consisting of H,

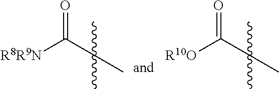

wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₅)alkyl;

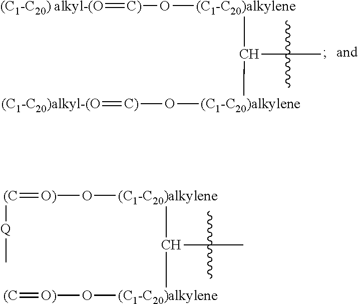

wherein Q is (C₃-C₁₅)alkylene;

wherein when R³ and R^{5′} are each H and X is F or Cl, both R¹ and R² cannot be H;

or a pharmaceutically acceptable salt thereof.

or Cl, both R¹ and R² cannot be H;

or a pharmaceutically acceptable salt thereof.

Preferably in embodiments of formulae (I)-(II), X is F.

Preferably in embodiments of formulae (I)-(II), R¹ and R² are each H.

Preferably in embodiments of formulae (I)-(II), R¹ is H and R² is selected from the group consisting of

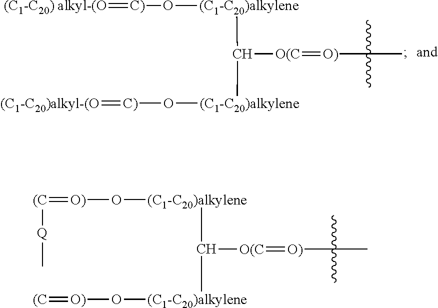

wherein Q is (C₃-C₁₅)alkylene;

Preferably, in one embodiment, R¹ is H and R² is:

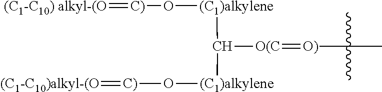

Preferably, in one embodiment, R¹ is H and R² is:

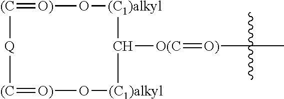

wherein Q is (C₃-C₁₅)alkylene.

Preferably in embodiments of formulae (I)-(II), R³ is H.

Preferably in embodiments of formulae (I)-(II), R³ is C(═O)—Z—R⁴, Z is a bond and R⁴ is NR⁵R⁶ wherein R⁵ is H or (C₁-C₆)alkyl and R⁶ is selected from the group consisting of (C₁-C₂₀)alkyl, (C₃-C₆)cycloalkyl; —(CH₂)ₘ—(C═O)—O—(C₁-C₂₀)alkyl wherein m is an integer ranging from 1 to 10; or wherein R⁵ and R⁶ join to form a (C₃-C₆)heterocycle. In various embodiments, R⁵ is H and R⁶ is (C₁-C₂₀)alkyl. In various embodiments, R⁵ is H and R⁶ is —(CH₂)ₘ—(C═O)—O—(C₁-C₂₀)alkyl, more preferably —(CH₂)—(C═O)—O—(C₁-C₆)alkyl. In various embodiments, R⁵ and R⁶ form a (C₃-C₆)heterocycle which may be optionally substituted by (C₁-C₆)alkyl. More preferably, R⁵ and R⁶ form a (C₆)heterocycle substituted by (C₁)alkyl. In one embodiment, R⁵ and R⁶ are each (C₁-C₆) alkyl, more preferably each (C₁-C₄)alkyl). In one embodiment, R⁵ is H and R⁶ is (C₃-C₆)cycloalkyl, more preferably (C₃)cycloalkyl.

In one embodiment, Z is —O— and R⁴ is:

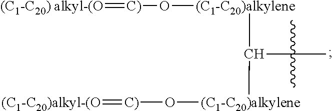

More preferably, Z is —O— and R⁴ is:

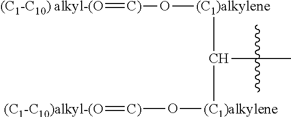

In one embodiment, Z is —O— and R⁴ is:

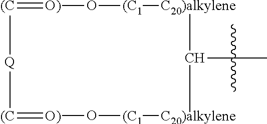

wherein Q is (C₃-C₁₅)alkylene;

More preferably, Z is —O— and R⁴ is:

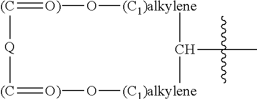

wherein Q is (C₁₀-C₁₅)alkylene.

In one embodiment, Z is —O— and R⁴ is —(C₁-C₆)alkylene-aryl-R¹¹, wherein R¹¹ is selected from H and —O(C═O)—(C₁-C₂₅)alkyl, preferably, aryl is (C₆-C₁₄)aryl. More preferably, R⁴ is —(C₁)alkylene-(C₆)aryl)(C₁₀-C₂₅)alkyl.

Preferably in embodiments of formulae (I)-(II), R^{5′} is H.

Preferably in embodiments of formulae (I)-(II), R^{5′} is:

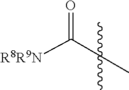

wherein R⁸ is H or (C₁-C₆)alkyl and R⁹ is (C₁-C₂₀)alkyl. In one embodiment, R⁸ is H and R⁹ is (C₁-C₂₀)alkyl. In one embodiment, R⁸ is (C₁-C₄)alkyl and R⁹ is (C₁-C₄)alkyl.

Preferably in embodiments of formulae (I)-(II), R^{5′} is:

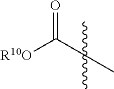

wherein R¹⁰ is selected from —(C₁-C₆)alkylene-aryl-R¹¹, (preferably aryl is (C₆-C₁₄)aryl, more preferably (C₆) wherein R¹¹ is selected from H and —O(C═O)—(C₁-C₂₀)alkyl;

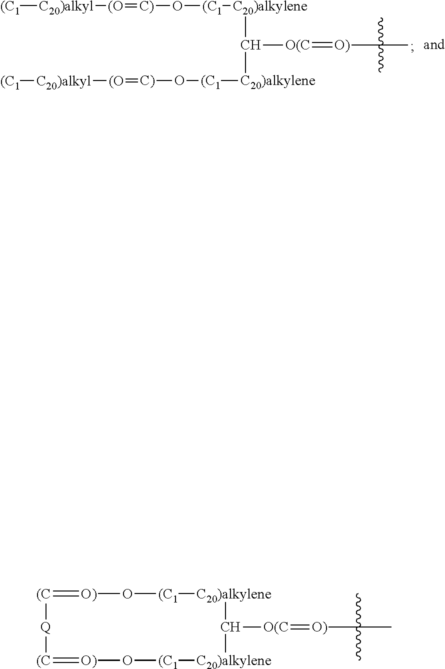

wherein Q is (C₃-C₁₅)alkylene;

In various embodiments, and in another aspect of the present invention, the invention may encompass various individual compounds. As an example, such specific compounds may be selected from the group consisting of (Table 1):

TABLE 1

| Example | Structure | Chemical Name |
|---|---|---|
| 1 | 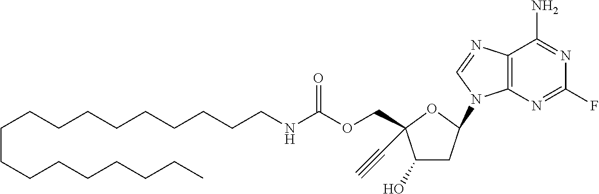 [SMILES: C#C[C@]1(COC(=O)NCCCCCCCCCCCCCCCCCC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl octadecylcarbamate |
| 2 | 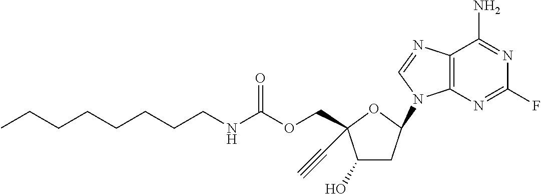 [SMILES: C#C[C@]1(COC(=O)NCCCCCCCC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl octylcarbamate |
| 3 | 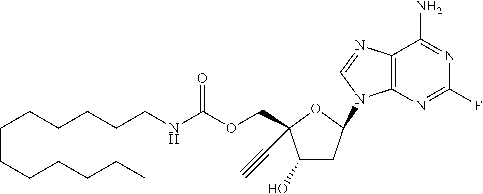 [SMILES: C#C[C@]1(COC(=O)NCCCCCCCCCCCC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl dodecylcarbamate |
| 4 | 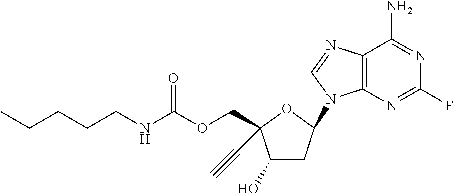 [SMILES: C#C[C@]1(COC(=O)NCCCCC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl pentylcarbamate |
| 5 | 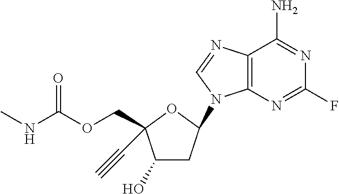 [SMILES: C#C[C@]1(COC(=O)NC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl methylcarbamate |
| 6 | 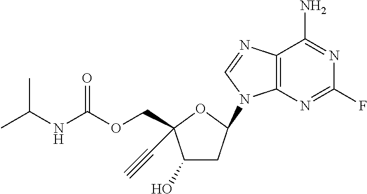 [SMILES: C#C[C@]1(COC(=O)NC(C)C)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl isopropylcarbamate |
| 7 | 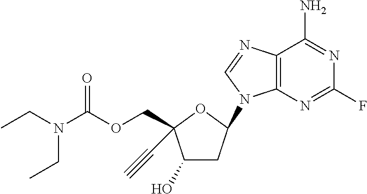 [SMILES: C#C[C@]1(COC(=O)N(CC)CC)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl diethylcarbamate |
| 8 | 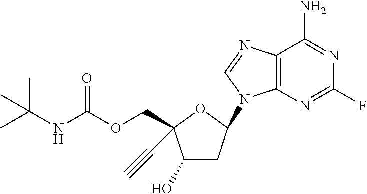 [SMILES: C#C[C@]1(COC(=O)NC(C)(C)C)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl tert-butylcarbamate |
| 9 | 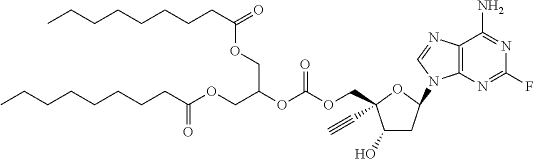 [SMILES: C#C[C@]1(COC(=O)OC(COC(=O)CCCCCCCC)COC(=O)CCCCCCCC)O[C@@H](n2cnc3c(N)nc(F)nc32)C[C@@H]1O] | 2-(((((2R,3S,5R)-5-(6-amino-2-fluoro- 9H-purin-9-yl)-2-ethynyl-3- hydroxytetrahydrofuran-2- yl)methoxy)carbonyl)oxy)propane-1,3- diyl dinonanoate |
| 10 | 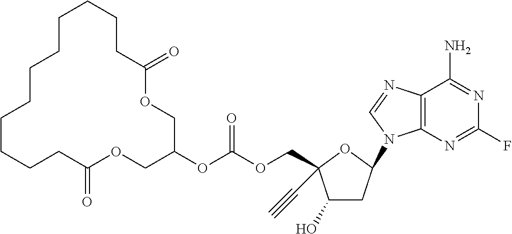 [SMILES: C#C[C@]1(COC(=O)OC2COC(=O)CCCCCCCCCCCC(=O)OC2)O[C@@H](n2cnc3c(N)nc(F)nc32)C[C@@H]1O] | ((2R,3S,5R)-5-(6-amino-2-fluoro-9H- purin-9-yl)-2-ethynyl-3- hydroxytetrahydrofuran-2-yl)methyl (6,18-dioxo-1,5-dioxacyclooctadecan- 3-yl)carbonate |
| 11 | 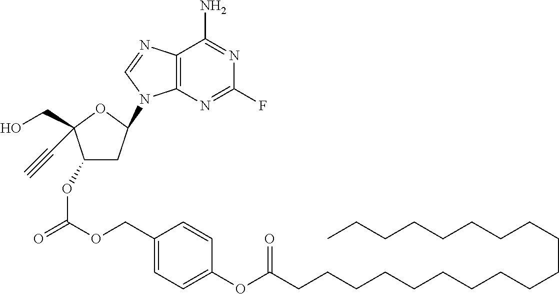 [SMILES: C#C[C@]1(CO)O[C@@H](n2cnc3c(N)nc(F)nc32)C[C@@H]1OC(=O)OCc1ccc(OC(=O)CCCCCCCCCCCCCCCCCCC)cc1] | 4-((((((2R,3S,5R)-5-(6-amino-2-fluoro- 9H-purin-9-yl)-2-ethynyl-2- (hydroxymethyl)tetrahydrofuran-3- yl)oxy)carbonyl)oxy)methyl)phenyl icosanoate |
| 12 | 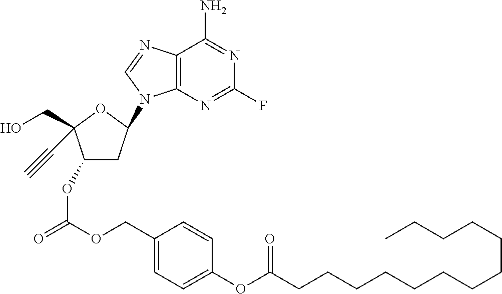 [SMILES: C#C[C@]1(CO)O[C@@H](n2cnc3c(N)nc(F)nc32)C[C@@H]1OC(=O)OCc1ccc(OC(=O)CCCCCCCCCCCCC)cc1] | 4-((((((2R,3S,5R)-5-(6-amino-2-fluoro- 9H-purin-9-yl)-2-ethynyl-2- (hydroxymethyl)tetrahydrofuran-3- yl)oxy)carbonyl)oxy)methyl)phenyl tetradecanoate |
| 13 | 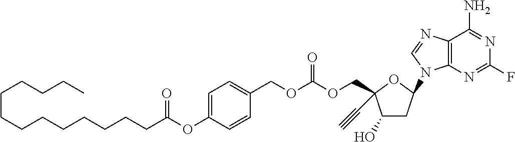 [SMILES: C#C[C@]1(COC(=O)OCc2ccc(OC(=O)CCCCCCCCCCCCC)cc2)O[C@@H](n2cnc3c(N)nc(F)nc32)C[C@@H]1O] | 4-((((((2R,3S,5R)-5-(6-amino-2-fluoro- 9H-purin-9-yl)-2-ethynyl-3- hydroxytetrahydrofuran-2- yl)methoxy)carbonyl)oxy)methyl)phenyl tetradecanoate |
| 14 | 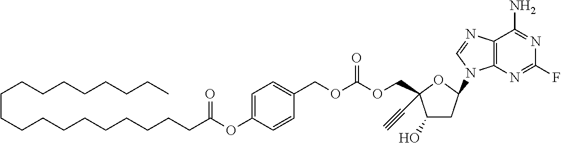 [SMILES: C#C[C@]1(COC(=O)OCc2ccc(OC(=O)CCCCCCCCCCCCCCCCCCC)cc2)O[C@@H](n2cnc3c(N)nc(F)nc32)C[C@@H]1O] | 4-((((((2R,3S,5R)-5-(6-amino-2-fluoro- 9H-purin-9-yl)-2-ethynyl-3- hydroxytetrahydrofuran-2- yl)methoxy)carbonyl)oxy)methyl)phenyl icosanoate |
| 15 | 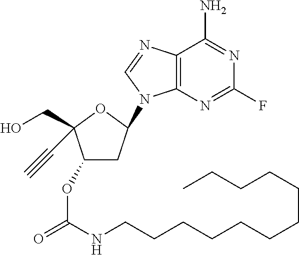 [SMILES: C#C[C@]1(CO)O[C@@H](n2cnc3c(N)nc(F)nc32)C[C@@H]1OC(=O)NCCCCCCCCCCCC] | (2R,3S,5R)-5-(6-amino-2-fluoro-9H- purin-9-yl)-2-ethynyl-2- (hydroxymethyl)tetrahydrofuran-3-yl dodecylcarbamate |
| 16 | 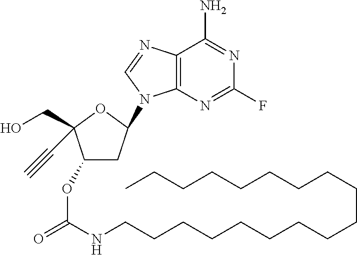 [SMILES: C#C[C@]1(CO)O[C@@H](n2cnc3c(N)nc(F)nc32)C[C@@H]1OC(=O)NCCCCCCCCCCCCCCCCCC] | (2R,3S,5R)-5-(6-amino-2-fluoro-9H- purin-9-yl)-2-ethynyl-2- (hydroxymethyl)tetrahydrofuran-3-yl octadecylcarbamate |
| 17 | 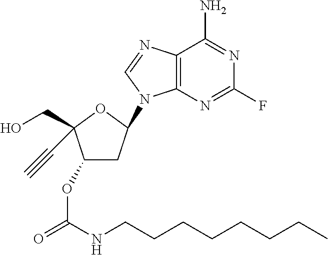 [SMILES: C#C[C@]1(CO)O[C@@H](n2cnc3c(N)nc(F)nc32)C[C@@H]1OC(=O)NCCCCCCCC] | (2R,3S,5R)-5-(6-amino-2-fluoro-9H- purin-9-yl)-2-ethynyl-2- (hydroxymethyl)tetrahydrofuran-3-yl octylcarbamate |
| 18 | 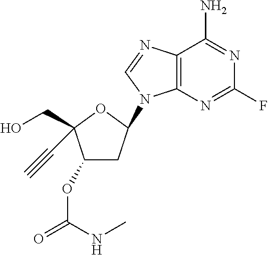 [SMILES: C#C[C@]1(CO)O[C@@H](n2cnc3c(N)nc(F)nc32)C[C@@H]1OC(=O)NC] | (2R,3S,5R)-5-(6-amino-2-fluoro-9H- purin-9-yl)-2-ethynyl-2- (hydroxymethyl)tetrahydrofuran-3-yl methylcarbamate |
| 19 | 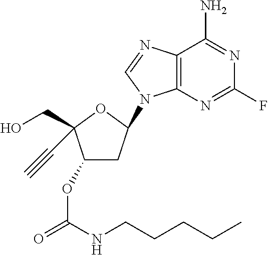 [SMILES: C#C[C@]1(CO)O[C@@H](n2cnc3c(N)nc(F)nc32)C[C@@H]1OC(=O)NCCCCC] | (2R,3S,5R)-5-(6-amino-2-fluoro-9H- purin-9-yl)-2-ethynyl-2- (hydroxymethyl)tetrahydrofuran-3-yl pentylcarbamate |
| 20 | 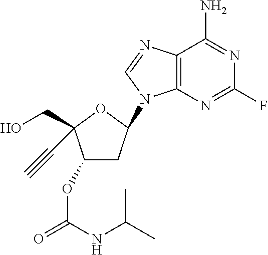 [SMILES: C#C[C@]1(CO)O[C@@H](n2cnc3c(N)nc(F)nc32)C[C@@H]1OC(=O)NC(C)C] | (2R,3S,5R)-5-(6-amino-2-fluoro-9H- purin-9-yl)-2-ethynyl-2- (hydroxymethyl)tetrahydrofuran-3-yl isopropylcarbamate |
| 21 | 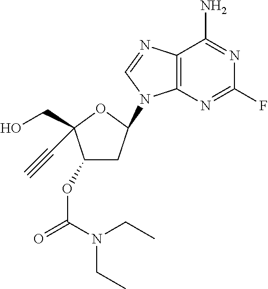 [SMILES: C#C[C@]1(CO)O[C@@H](n2cnc3c(N)nc(F)nc32)C[C@@H]1OC(=O)N(CC)CC] | (2R,3S,5R)-5-(6-amino-2-fluoro-9H- purin-9-yl)-2-ethynyl-2- (hydroxymethyl)tetrahydrofuran-3-yl diethylcarbamate |
| 22 | 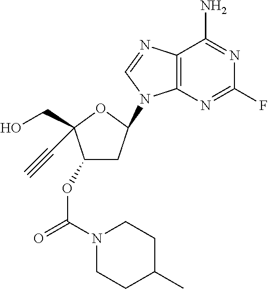 [SMILES: C#C[C@]1(CO)O[C@@H](n2cnc3c(N)nc(F)nc32)C[C@@H]1OC(=O)N1CCC(C)CC1] | (2R,3S,5R)-5-(6-amino-2-fluoro-9H- purin-9-yl)-2-ethynyl-2- (hydroxymethyl)tetrahydrofuran-3-yl 4- methylpiperidine-1-carboxylate |
| 23 | 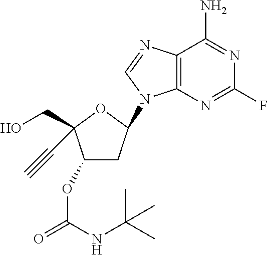 [SMILES: C#C[C@]1(CO)O[C@@H](n2cnc3c(N)nc(F)nc32)C[C@@H]1OC(=O)NC(C)(C)C] | (2R,3S,5R)-5-(6-amino-2-fluoro-9H- purin-9-yl)-2-ethynyl-2- (hydroxymethyl)tetrahydrofuran-3-yl tert-butylcarbamate |
| 24 | 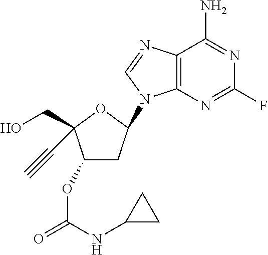 [SMILES: C#C[C@]1(CO)O[C@@H](n2cnc3c(N)nc(F)nc32)C[C@@H]1OC(=O)NC1CC1] | (2R,3S,5R)-5-(6-amino-2-fluoro-9H- purin-9-yl)-2-ethynyl-2- (hydroxymethyl)tetrahydrofuran-3-yl cyclopropylcarbamate |
| 25 | 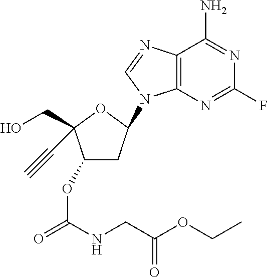 [SMILES: C#C[C@]1(CO)O[C@@H](n2cnc3c(N)nc(F)nc32)C[C@@H]1OC(=O)NCC(=O)OCC] | ethyl((((2R,3S,5R)-5-(6-amino-2- fluoro-9H-purin-9-yl)-2-ethynyl-2- (hydroxymethyl)tetrahydrofuran-3- yl)oxy)carbonyl)glycinate |
| 26 | 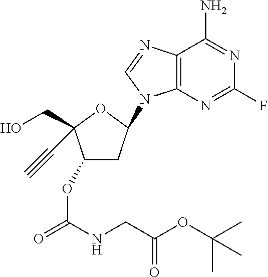 [SMILES: C#C[C@]1(CO)O[C@@H](n2cnc3c(N)nc(F)nc32)C[C@@H]1OC(=O)NCC(=O)OC(C)(C)C] | tert-butyl((((2R,3S,5R)-5-(6-amino-2- fluoro-9H-purin-9-yl)-2-ethynyl-2- (hydroxymethyl)tetrahydrofuran-3- yl)oxy)carbonyl)glycinate |
| 27 | 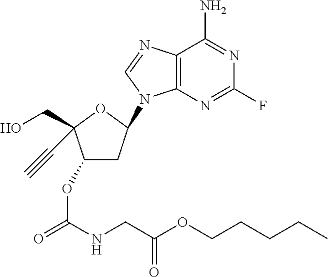 [SMILES: C#C[C@]1(CO)O[C@@H](n2cnc3c(N)nc(F)nc32)C[C@@H]1OC(=O)NCC(=O)OCCCCC] | pentyl((((2R,3S,5R)-5-(6-amino-2- fluoro-9H-purin-9-yl)-2-ethynyl-2- (hydroxymethyl)tetrahydrofuran-3- yl)oxy)carbonyl)glycinate |
| 28 | 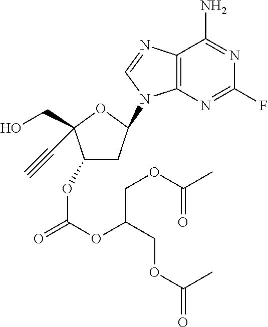 [SMILES: C#C[C@]1(CO)O[C@@H](n2cnc3c(N)nc(F)nc32)C[C@@H]1OC(=O)OC(COC(C)=O)COC(C)=O] | 2-(((((2R,3S,5R)-5-(6-amino-2-fluoro- 9H-purin-9-yl)-2-ethynyl-2- (hydroxymethyl)tetrahydrofuran-3- yl)oxy)carbonyl)oxy)propane-1,3-diyl diacetate |
| 29 | 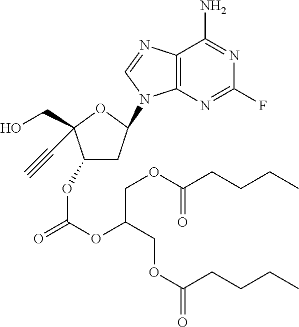 [SMILES: C#C[C@]1(CO)O[C@@H](n2cnc3c(N)nc(F)nc32)C[C@@H]1OC(=O)OC(COC(=O)CCCC)COC(=O)CCCC] | 2-(((((2R,3S,5R)-5-(6-amino-2-fluoro- 9H-purin-9-yl)-2-ethynyl-2- (hydroxymethyl)tetrahydrofuran-3- yl)oxy)carbonyl)oxy)propane-1,3-diyl dipentanoate |
| 30 | 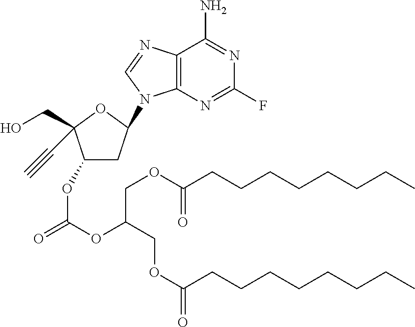 [SMILES: C#C[C@]1(CO)O[C@@H](n2cnc3c(N)nc(F)nc32)C[C@@H]1OC(=O)OC(COC(=O)CCCCCCCC)COC(=O)CCCCCCCC] | 2-(((((2R,3S,5R)-5-(6-amino-2-fluoro- 9H-purin-9-yl)-2-ethynyl-2- (hydroxymethyl)tetrahydrofuran-3- yl)oxy)carbonyl)oxy)propane-1,3-diyl dinonanoate |
| 31 | 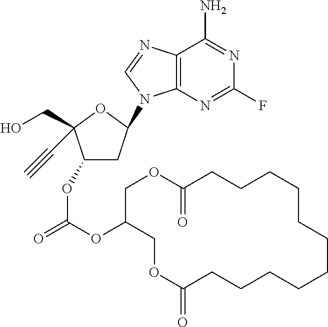 [SMILES: C#C[C@]1(CO)O[C@@H](n2cnc3c(N)nc(F)nc32)C[C@@H]1OC(=O)OC1COC(=O)CCCCCCCCCCCC(=O)OC1] | (2R,3S,5R)-5-(6-amino-2-fluoro-9H- purin-9-yl)-2-ethynyl-2- (hydroxymethyl)tetrahydrofuran-3-yl (6,18-dioxo-1,5-dioxacyclooctadecan- 3-yl)carbonate |
| 32 | 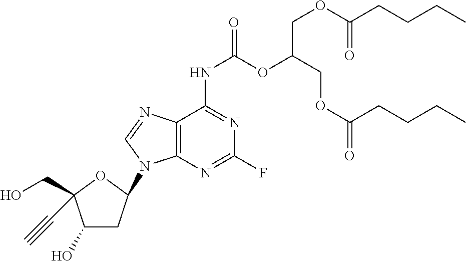 [SMILES: C#C[C@]1(CO)O[C@@H](n2cnc3c(NC(=O)OC(COC(=O)CCCC)COC(=O)CCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6- yl)carbamoyl)oxy)propane-1,3-diyl dipentanoate |
| 33 | 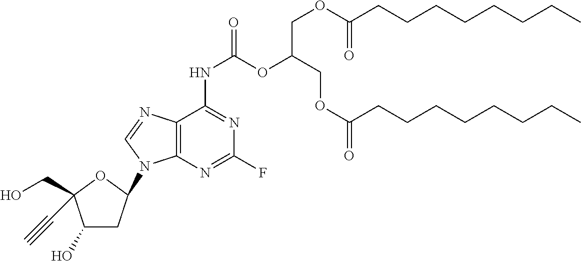 [SMILES: C#C[C@]1(CO)O[C@@H](n2cnc3c(NC(=O)OC(COC(=O)CCCCCCCC)COC(=O)CCCCCCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6- yl)carbamoyl)oxy)propane-1,3-diyl dinonanoate |
| 34 | 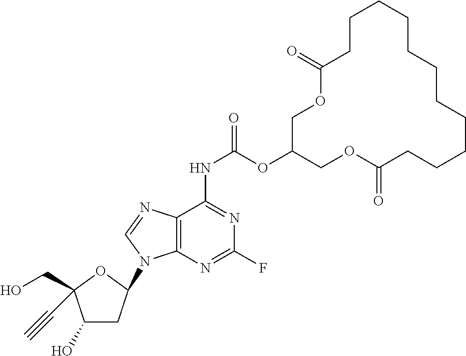 [SMILES: C#C[C@]1(CO)O[C@@H](n2cnc3c(NC(=O)OC4COC(=O)CCCCCCCCCCCC(=O)OC4)nc(F)nc32)C[C@@H]1O] | 6,18-dioxo-1,5-dioxacyclooctadecan-3- yl(9-((2R,4S,5R)-5-ethynyl-4-hydroxy- 5-(hydroxymethyl)tetrahydrofuran-2-yl)- 2-fluoro-9H-purin-6-yl)carbamate |
| 35 | 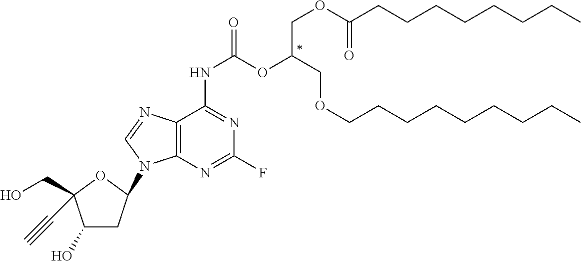 [SMILES: C#C[C@]1(CO)O[C@@H](n2cnc3c(NC(=O)OC(COCCCCCCCCC)COC(=O)CCCCCCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6-yl)carbamoyl)oxy)-3- (nonyloxy)propyl nonanoate diastereomer 1 |
| 36 | 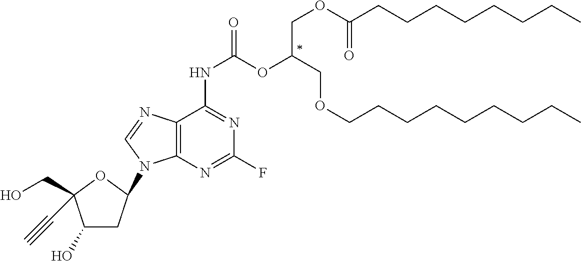 [SMILES: C#C[C@]1(CO)O[C@@H](n2cnc3c(NC(=O)OC(COCCCCCCCCC)COC(=O)CCCCCCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6-yl)carbamoyl)oxy)-3- (nonyloxy)propyl nonanoate diastereomer 2 |
| 37 | 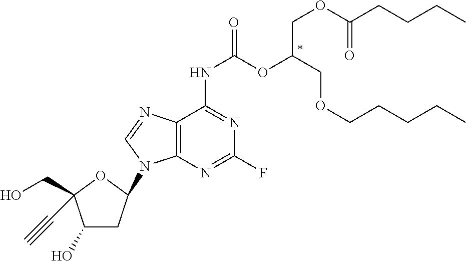 [SMILES: C#C[C@]1(CO)O[C@@H](n2cnc3c(NC(=O)OC(COCCCCC)COC(=O)CCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6-yl)carbamoyl)oxy)-3- (pentyloxy)propyl pentanoate diastereomer 1 |
| 38 | 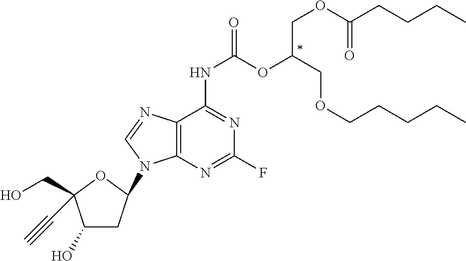 [SMILES: C#C[C@]1(CO)O[C@@H](n2cnc3c(NC(=O)OC(COCCCCC)COC(=O)CCCC)nc(F)nc32)C[C@@H]1O] | 2-(((9-((2R,4S,5R)-5-ethynyl-4- hydroxy-5- (hydroxymethyl)tetrahydrofuran-2-yl)-2- fluoro-9H-purin-6-yl)carbamoyl)oxy)-3- (pentyloxy)propyl pentanoate diastereomer 2 |

and pharmaceutically acceptable salts thereof.

In one embodiment, the present invention encompasses each individual compound listed in the above Table 1, or a pharmaceutically acceptable salt thereof.

In various embodiments, prodrugs of any of the compounds of formulae (I)-(II) set forth herein are also within the scope of the present invention.

In accordance with one embodiment of the present invention, there is provided a pharmaceutical composition comprising a compound of Formulae (I)-(II) or a pharmaceutically acceptable salt thereof, and a pharmaceutically acceptable excipient. In a further embodiment, the compound is present in amorphous form. In a further embodiment, the pharmaceutical composition is in a tablet form. In a further embodiment, the pharmaceutical composition is in parenteral form. In a further embodiment, the compound is present as a spray dried dispersion.

In accordance with one embodiment of the present invention, there is provided a method of treating an HIV infection in a subject comprising administering to the subject a compound of Formulae (I)-(II) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulae (I)-(II) or a pharmaceutically acceptable salt thereof.

In accordance with one embodiment of the present invention, there is provided the use of a compound of Formulae (I)-(II) in the manufacture of a medicament for treating an HIV infection.

In accordance with one embodiment of the present invention, there is provided the use of a compound of Formulae (I)-(II) in the manufacture of a medicament for preventing an HIV infection.

In accordance with one embodiment of the present invention, there is provided a compound according to Formulae (I)-(II) for use in treating an HIV infection.

In accordance with one embodiment of the present invention, there is provided a compound according to Formulae (I)-(II) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formulae (I)-(II) wherein the compound or salt of the compound is used in the manufacture of a medicament for use in the treatment of an HIV infection in a human.

In another embodiment of the invention, there is provided a compound of Formulae (I)-(II) wherein the compound or salt of the compound is used in the manufacture of a medicament for use in the prevention of an HIV infection in a human.

In one embodiment, the pharmaceutical formulation containing a compound of Formulae (I)-(II) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formulae (I)-(II) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, vicriviroc (Sch-C), Sch-D, TAK779, maraviroc, TAK449, didanosine, tenofovir, lopinavir, and darunavir.

As such, the compounds of the present invention of Formulae (I)-(II) and any other pharmaceutically active agent(s) may be administered together or separately and, when administered separately, administration may occur simultaneously or sequentially, in any order. The amounts of the compounds of Formulae (I)-(II) of the present invention and the other pharmaceutically active agent(s) and the relative timings of administration will be selected in order to achieve the desired combined therapeutic effect. The administration in combination of a compound of the present invention of Formulae (I)-(II) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formulae (I)-(II) or salts thereof and the other pharmaceutically active agent(s) and the relative timings of administration will be selected in order to achieve the desired combined therapeutic effect.

In addition, the compounds of the present invention of Formulae (I)-((I) may be used in combination with one or more other agents that may be useful in the prevention or treatment of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068, BMS-626529, 5-Helix and similar agents;
Integrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/USO3/39619, PCT/USO3/39618, PCT/US03/39740, and PCT/US03/39732, and similar agents.

Other combinations may be used in conjunction with the compounds of the present invention, e.g., Biktarvy (Bictegravir/Emtricitabine/Tenofovir/Alafenamide) made commercially available by Gilead Sciences

Further examples where the compounds of the present invention may be used in combination with one or more agents useful in the prevention or treatment of HIV are found in Table 2.

TABLE 2

| | | | |
|---|---|---|---|
| | Brand | | |
| FDA Approval | Name | Generic Name | Manufacturer |

Nucleoside Reverse Transcriptase Inhibitors (NRTIs)

| | | | |
|---|---|---|---|
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, | Squibb |
| | | ddl | |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddl | Squibb |
| | | EC | |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |

Non-Nucleosides Reverse Transcriptase Inhibitors (NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |

Protease Inhibitors (Pls)

| | | | |
|---|---|---|---|
| 1995 | Invirase | saquinavir | Roche |
| | | mesylate, SQV | Pharmaceuticals |
| 1996 | Norvir | ritonavir, RTV | Abbott |
| | | | Laboratories |
| 1996 | Crixivan | indinavir, IDV | Merck |
| 1997 | Viracept | nelfinavir | Pfizer |
| | | mesylate, NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + | Abbott |
| | | ritonavir, LPV/RTV | Laboratories |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals & |
| | | | Trimeris |

Entry Inhibitors

| | | | |
|---|---|---|---|
| 2007 | Selzentry | Maraviroc | Pfizer |

Integrase Inhibitors

| | | | |
|---|---|---|---|
| 2007 | Isentress | Raltegravir | Merck |
| 2013 | Tivicay | Dolutegravir | ViiV Healthcare |
| — | — | Cabotegravir | |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.
SPI-452 is a compound being developed by Sequoia Pharmaceuticals of Gaithersburg, Md., as a pharmacological enhancer.

In one embodiment of the present invention, a compound of Formulae (I)-(II) is used in combination with ritonavir. In one embodiment, the combination is an oral fixed dose combination. In another embodiment, the compound of Formulae (I)-(II) is formulated as a long acting parenteral injection and ritonavir is formulated as an oral composition. In one embodiment, a kit containing the compound of Formulae (I)-(II) is formulated as a long acting parenteral injection and ritonavir formulated as an oral composition. In another embodiment, the compound of Formulae (I)-(II) is formulated as a long acting parenteral injection and ritonavir is formulated as an injectable composition. In one embodiment, a kit containing the compound of Formulae (I)-(II) is formulated as a long acting parenteral injection and ritonavir formulated as an injectable composition.

In another embodiment of the present invention, a compound of Formula (I)-(II) is used in combination with GS-9350. In one embodiment, the combination is an oral fixed dose combination. In another embodiment, the compound of Formulae (I)-(II) is formulated as a long acting parenteral injection and GS-9350 is formulated as an oral composition. In one embodiment, there is provided a kit containing the compound of Formulae (I)-(II) is formulated as a long acting parenteral injection and GS-9350 formulated as an oral composition. In another embodiment, the compound of Formulae (I)-(II) is formulated as a long acting parenteral injection and GS-9350 is formulated as an injectable composition. In one embodiment, is a kit containing the compound of Formulae (I)-(II) is formulated as a long acting parenteral injection and GS-9350 formulated as an injectable composition.

In one embodiment of the present invention, a compound of Formulae (I)-(II) is used in combination with SPI-452. In one embodiment, the combination is an oral fixed dose combination. In another embodiment, the compound of Formulae (I)-(II) is formulated as a long acting parenteral injection and SPI-452 is formulated as an oral composition. In one embodiment, there is provided a kit containing the compound of Formulae (I)-(II). formulated as a long acting parenteral injection and SPI-452 formulated as an oral composition. In another embodiment, the compound of Formulae (I)-(II) is formulated as a long acting parenteral injection and SPI-452 is formulated as an injectable composition. In one embodiment, there is provided a kit containing the compound of Formulae (I)-(II) formulated as a long acting parenteral injection and SPI-452 formulated as an injectable composition.

In one embodiment of the present invention, a compound of Formulae (I)-(II) is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I)-(II).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I)-(II), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I)-(II) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I)-(II).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I)-(II), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I)-(II), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I)-(II) further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In further embodiments, the compound of the present invention of Formulae (I)-(II) or a pharmaceutically acceptable salt thereof, is selected from the group of compounds set forth in Table 1 above.

The compounds of Table 1 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formulae (I)-(II) or a pharmaceutically acceptable salt thereof.

In certain embodiments, the compound(s) of the present invention, or a pharmaceutically acceptable salt thereof, is chosen from the compounds set forth in Table 1. The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formulae (I)-(II) may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, p-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

The compounds of Formulae (I)-(II) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formulae (I)-(II) containing one or more asymmetric carbon atoms can exist as two or more stereoisomers. Where a compound of Formulae (I)-(II) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formulae (I)-(II), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formulae (I)-(II) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formulae (I)-(II) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formulae (I)-(II), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formulae (I)-(II) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. In one embodiment, the compounds of the invention are prodrugs of 4′-ethylnyl-2-fluoro-2′-deoxyadenosine (EFdA) disclosed e.g., in U.S. Pat. No. 7,339,053, which is a nucleoside reverse transcriptase inhibitor of the formula:

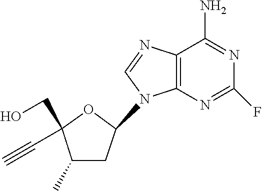 [SMILES: C#C[C@]1(CO)O[C@@H](n2cnc3c(N)nc(F)nc32)C[C@@H]1C]

The prodrugs are useful in that they are capable of modulating physicochemical properties, facilitating multiple dosing paradigms and improving pharmacokinetic and/or pharmacodynamic profiles of the active parent (EfdA). More specifically, EFdA has a relatively high aqueous solubility, rendering it unsuitable for slow release, long acting, parenteral dosing. Advantageously, prodrugs of EFdA of the invention are capable of having substantially reduced aqueous solubilities, that in some cases, may facilitate a slow release, parenteral dosing modality. Additionally, prodrugs of EFdA, of the invention may also reduce or eliminate undesirable injection site reactions associated with high localized concentrations of EFdA that occur upon parenteral dosing of EFdA itself. Moreover, prodrugs of EFdA of the invention may also, in some cases, confer an enhancement in antiviral persistence as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formulae (I)-(II), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may or take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates Described herein can be affected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### Examples and General Synthesis

The following examples and prophetic synthesis methods serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=aqueous
μL=microliters
μM=micromolar
NMR=nuclear magnetic resonance
Boc=tert-butoxycarbonyl
br=broad
Cbz=benzyloxycarbonyl
d=doublet
° C.=degrees Celsius
DCM=dichloromethane
dd=doublet of doublets
DIEA=N,N-diisopropylethylamine
DMAP=N,N-dimethylaminopyridine
DMEM=Dulbeco's Modified Eagle's Medium
DMF=N,N-dimethylformamide
DMSO=dimethylsufoxide
EDC=N-(3-Dimethylaminopropyl)-N′-ethylcarbodiimide hydrochloride
EtOAc=ethyl acetate
g=gram
h or hr=hour(s)
HPLC=high performance liquid chromatography
Hz=Hertz
IU=International Units
IC₅₀=50% inhibitory concentration
J=coupling constant in Hz
LCMS=liquid chromatography mass spectrometry
m=multiplet
M=molar concentration
M+H⁺=parent mass spectrum peak plus H⁺
mg=miligram
min=minute(s)
mL=milliliter
mM=millimolar
mmol=millimole
MMTr=monomethoxytrityl
MS=mass spectrum
MTBE=methyl tert-butyl ether
nM=nanomolar
PE=petroleum ether
ppm=parts per million
q.s.=sufficient amount
s=singlet
RT=room temperature
sat.=saturated
t=triplet
TBDMS=tert-butyldimethylsilyl
TBDPS=tert-butyldiphenylsilyl
TEA=triethylamine
THF=tetrahydrofuran
TMS=trimethylsilyl

Additionally, various compounds of the invention may be made, in one embodiment, by way of the general synthesis routes set forth in Schemes 1-6 below:

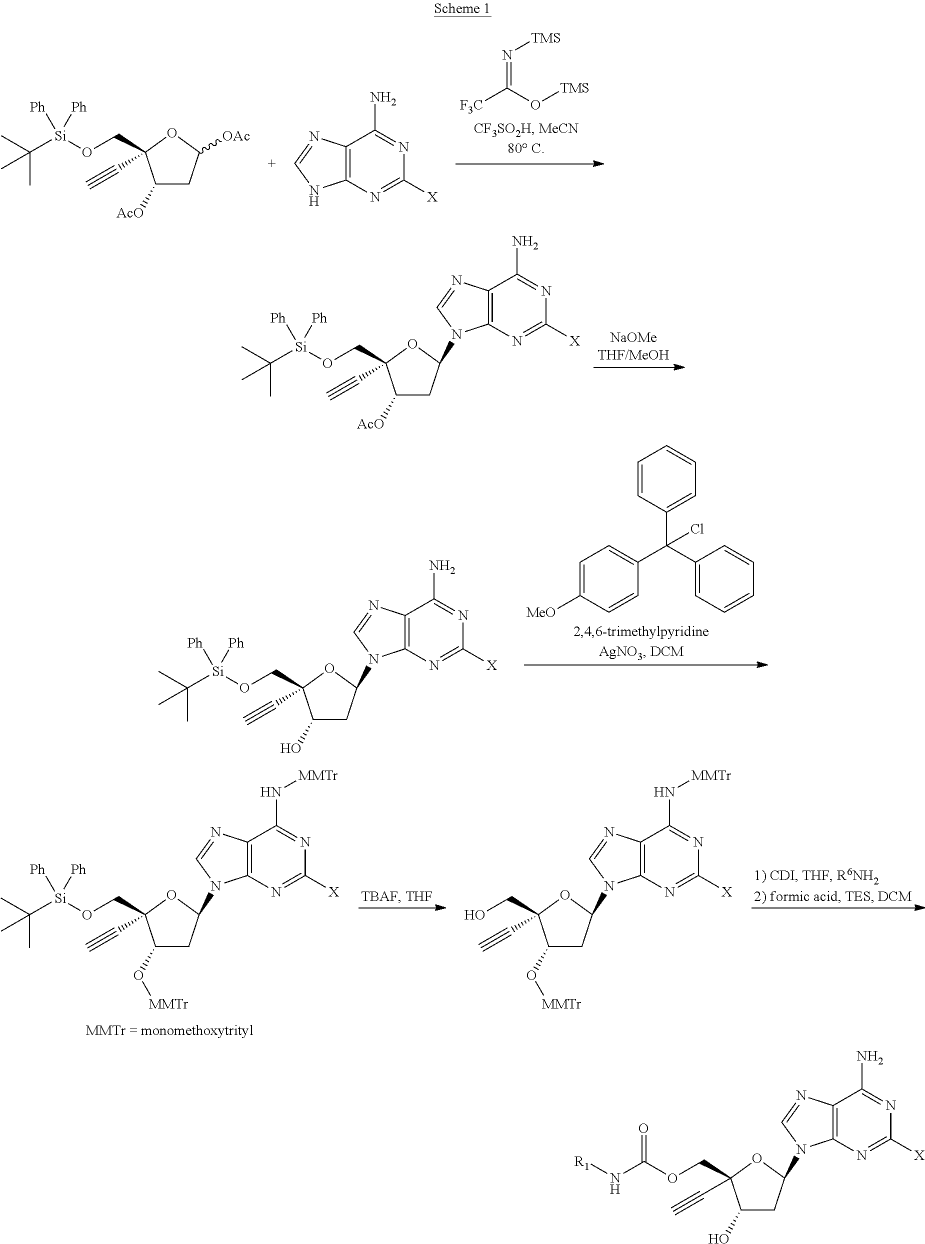

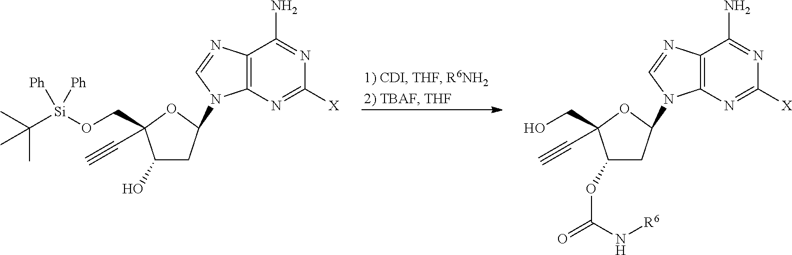

wherein X and R⁶ are defined herein.

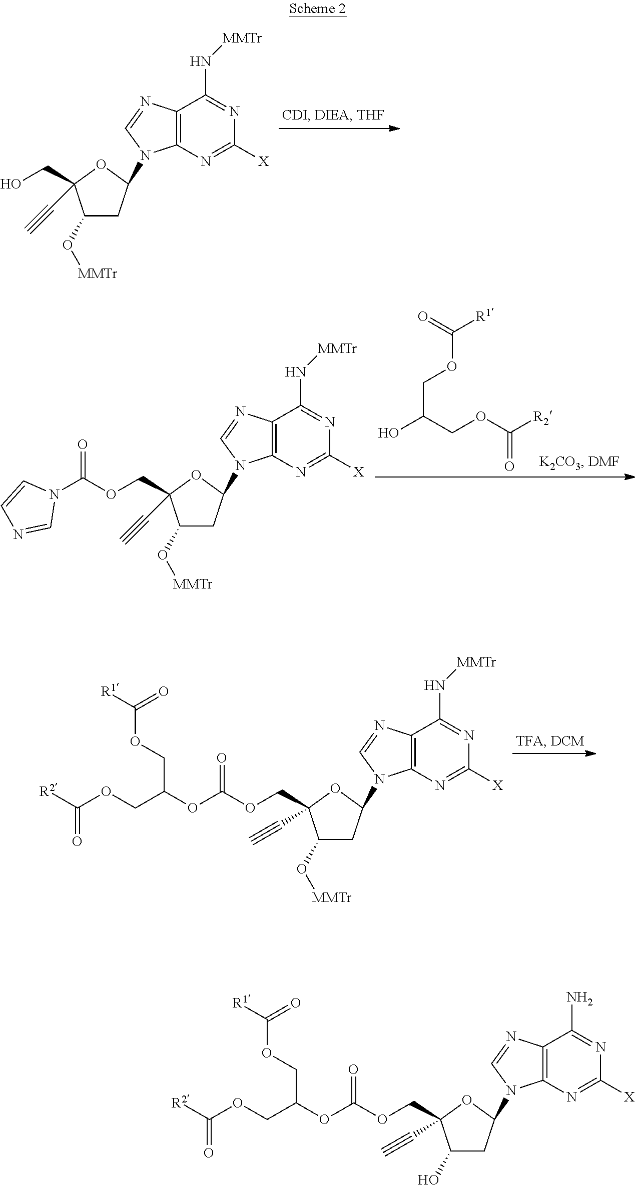

wherein X is defined herein, and R^{1′} and R^{2′} are independently selected from C₁ to C₂₀ alkyl.

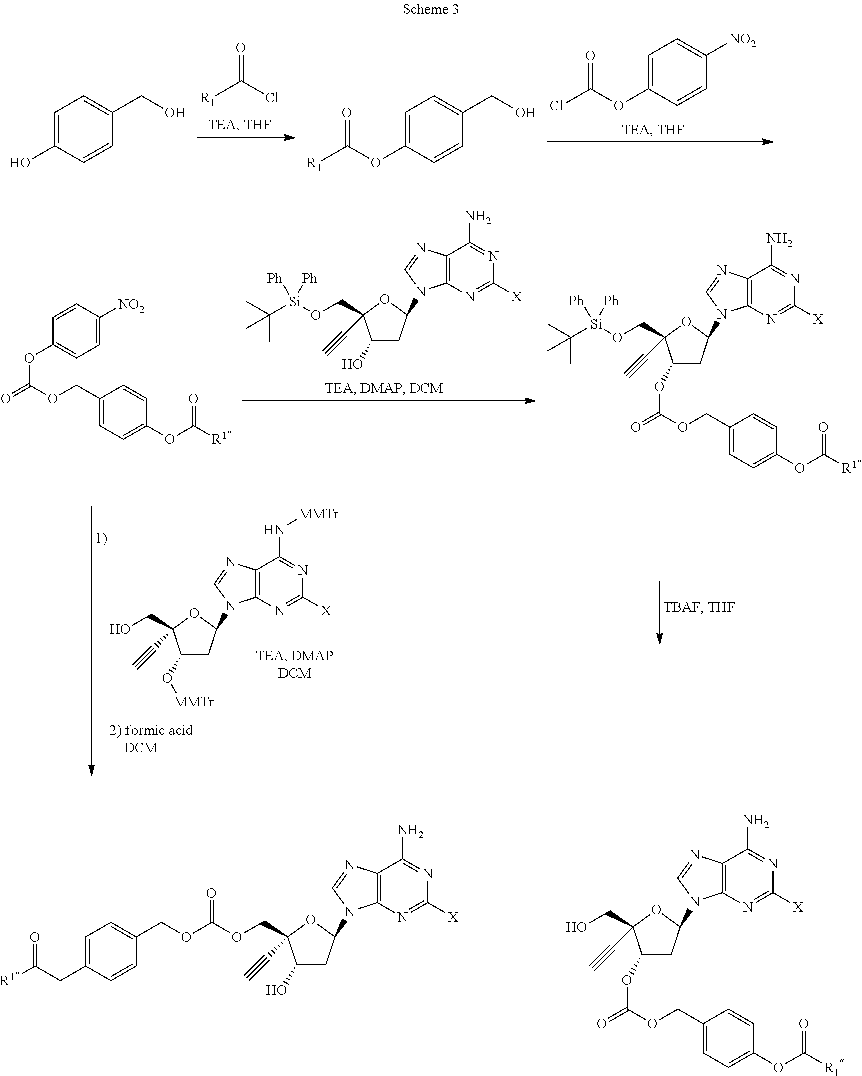

wherein X is defined herein, and R^{1″} is (C₁-C₂₀)alkyl.

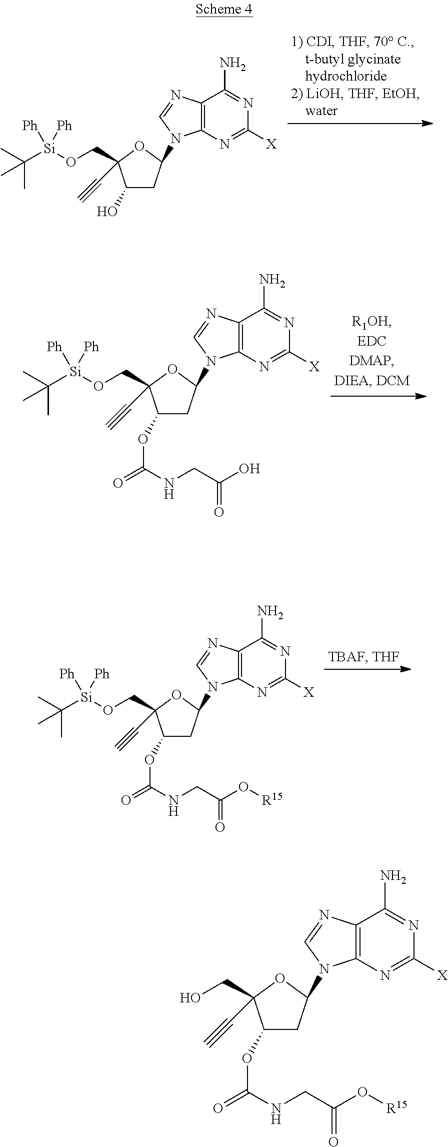

wherein X is defined hereinabove, and R¹⁵ is (C₁-C₂₀)alkyl.

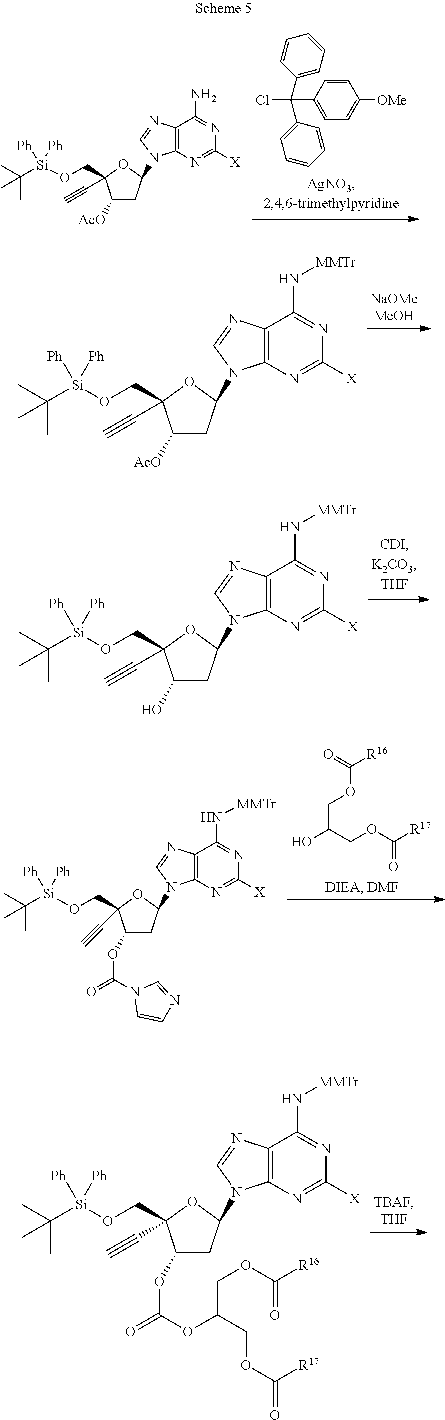

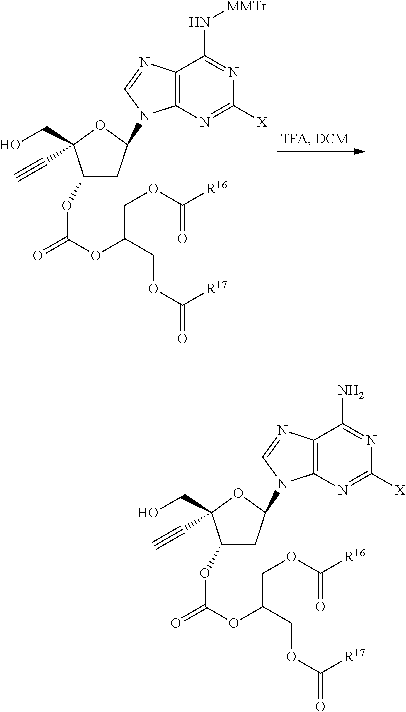

wherein X is defined hereinabove, and R¹⁶ and R¹⁷ are independently selected from (C₁-C₂₀) alkyl.

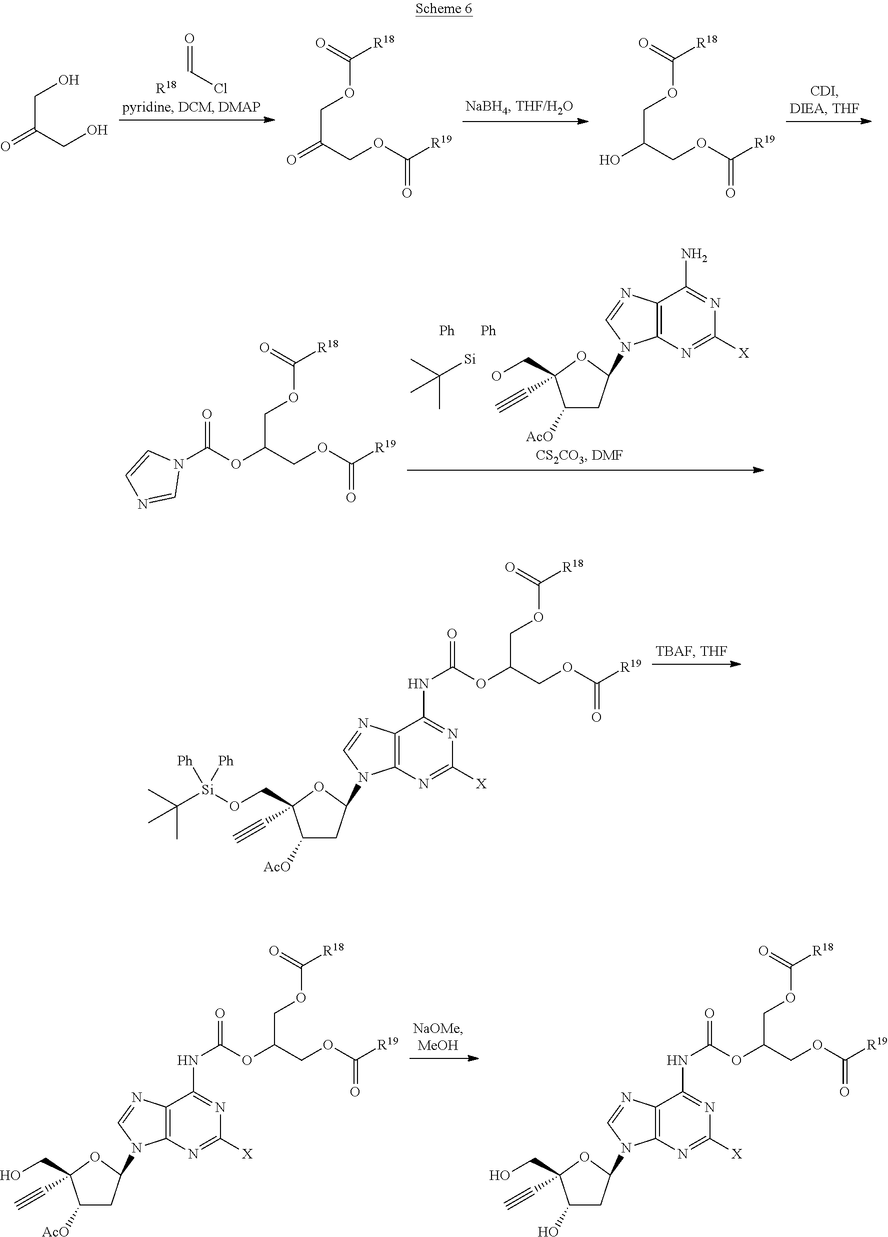

wherein X is defined hereinabove, and R¹⁸ and R¹⁹ are independently selected from (C₁-C₂₀) alkyl.
wherein:
Ac=acetyl
AcO=acetate
AgNO₃=silver nitrate
Cs₂CO₃=cesium carbonate
CDI=carbonyl diimidazole
DCM=dichloromethane

### DIEA=N,N-diisopropylethylamine

DMAP=4-dimethylaminopyridine

### DMF=N,N-dimethylformamide

EDC=1-ethyl-3-(3-dimethylaminopropyl) carbodiimide hydrochloride
EtOH—ethanol
K₂CO₃=potassium carbonate
LiOH=lithium hydroxide
MeCN=acetonitrile
MeOH=methanol
NaBH₄=sodium borohydride
NaOMe=sodium methoxide
Ph=phenyl
TEA=triethylamine
TES=triethylsilane
TBAF=tetra-n-butyl ammonium fluoride
TFA=trifluoroacetic acid
THF=tetrahydrofuran
and wherein X and R-groups are defined hereinabove

### Equipment Description

¹H NMR spectra were recorded on Varian or Bruker spectrometers. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Representative equipment and conditions for acquiring analytical low resolution LCMS are described below.

### Instrumentation:

Waters Acquity UPLC-MS system with SQ detectors

### MS Conditions:

Scan Mode: Alternating positive/negative electrospray

### Scan Range: 125-1200 amu

Scan Time: 150 msec
Interscan Delay: 50 msec

### LC Conditions:

The UPLC analysis was conducted on a Phenomenex Kinetex 1.7 um
2.1×50 mm XB-C18 column at 4° C.
0.2 uL of sample was injected using PLNO (partial loop with needle overfill) injection mode.
The gradient employed was:

### Mobile Phase A: Water+0.2% v/v Formic Acid

### Mobile Phase B: Acetonitrile+0.15% v/v Formic Acid

| Time | % A | % B | Flow Rate |
|---|---|---|---|
| 0.00 min | 95 | 5 | 1 ml/min |
| 1.1 min | 1 | 99 | 1 ml/min |
| 1.5 min | 1 | 99 | 1 ml/min |

UV detection provided by summed absorbance signal from 210 to 350 nm scanning at 40 Hz

### Example 1: ((2R,3S,5R)-5-(6-amino-2-fluoro-9H-purin-9-yl)-2-ethynyl-3-hydroxytetrahydrofuran-2-yl)methyl octadecylcarbamate

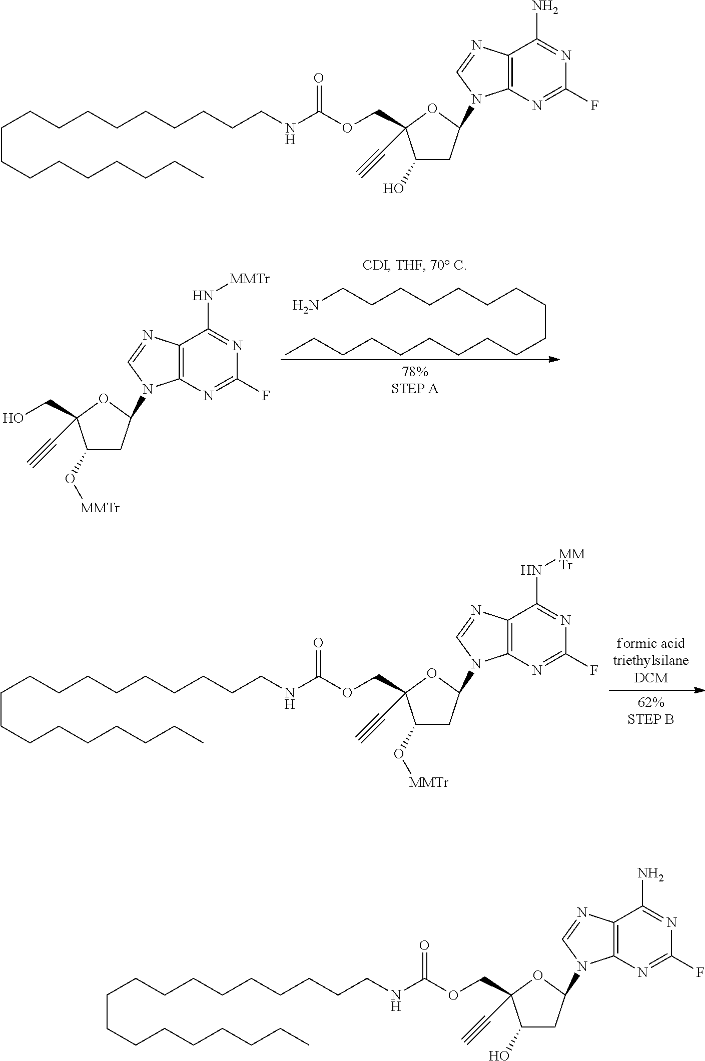

Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl octadecylcarbamate. To a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (71 mg, 0.085 mmol) in THF (1.0 mL) was added CDI (34.3 mg, 0.212 mmol) and the mixture was allowed to stir at 70° C. for 1 h. The mixture was cooled slightly, treated with octadecan-1-amine (91 mg, 0.339 mmol) and then stirred at ambient temperature for overnight. The mixture was concentrated and purified on silica gel (0-50% EtOAc/DCM) to afford the title compound (75 mg, 78%) as a white solid.

Step B: ((2R,3S,5R)-5-(6-amino-2-fluoro-9H-purin-9-yl)-2-ethynyl-3-hydroxytetrahydrofuran-2-yl)methyl octadecylcarbamate. To a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl octadecylcarbamate (75 mg, 0.066 mmol) in DCM (0.8 mL) was added formic acid (0.2 mL), followed by triethylsilane (0.021 mL, 0.132 mmol). The resulting orange solution was stirred for 60 minutes and then concentrated. The residue was purified on silica gel (0-10% MeOH/DCM) to afford the title compound (24 mg, 62%) as a white solid. LCMS (ESI) m/z calcd for C₃₁H₄₉FN₆O₄: 588.4. Found: 589.6 (M+1)⁺. ¹H NMR (400 MHz, METHANOL-d₄) δ 8.14 (s, 1H), 6.32 (dd, J=4.3, 7.4 Hz, 1H), 4.78-4.72 (m, 1H), 4.47 (d, J=11.7 Hz, 1H), 4.23 (d, J=11.9 Hz, 1H), 3.14 (s, 1H), 3.06-3.00 (m, 2H), 2.86-2.74 (m, 1H), 2.69-2.58 (m, 1H), 1.49-1.15 (m, 32H), 0.95-0.83 (m, 3H).

### Example 2: ((2R,3S,5R)-5-(6-amino-2-fluoro-9H-purin-9-yl)-2-ethynyl-3-hydroxytetrahydrofuran-2-yl)methyl octylcarbamate

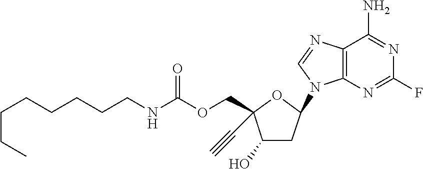 [SMILES: C#C[C@]1(COC(=O)NCCCCCCCC)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting octan-1-amine for octadecan-1-amine is step A. LCMS (ESI) m/z calcd for C₂₁H₂₉FN₆O₄: 448.2. Found: 449.3 (M+1)⁺. ¹H NMR (400 MHz, DMSO-d₆) δ 8.19-8.33 (m, 1H), 7.69-7.91 (m, 2H), 7.18 (br t, J=5.5 Hz, 1H), 6.25 (dd, J=7.6, 5.0 Hz, 1H), 5.71 (d, J=5.3 Hz, 1H), 4.51-4.61 (m, 1H), 4.35 (d, J=11.4 Hz, 1H), 4.04 (d, J=11.4 Hz, 1H), 3.57 (s, 1H), 2.85-3.00 (m, 2H), 2.71-2.81 (m, 1H), 2.56-2.41 (m, 1H, overlapping DMSO peak), 1.10-1.46 (m, 12H), 0.76-0.91 (m, 3H).

### Example 3: ((2R,3S,5R)-5-(6-amino-2-fluoro-9H-purin-9-yl)-2-ethynyl-3-hydroxytetrahydrofuran-2-yl)methyl dodecylcarbamate

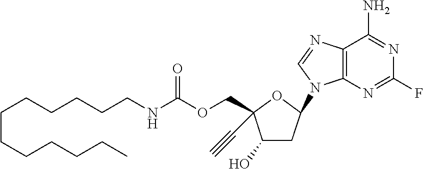 [SMILES: C#C[C@]1(COC(=O)NCCCCCCCCCCCC)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting dodecan-1-amine for octadecan-1-amine is step A. LCMS (ESI) m/z calcd for C₂₅H₃₇FN₆O₄: 504.3. Found: 505.4 (M+1)⁺. ¹H NMR (400 MHz, DMSO-d₆) δ 8.17-8.33 (m, 1H), 7.81 (br s, 2H), 7.18 (br t, J=5.7 Hz, 1H), 6.25 (dd, J=7.5, 4.9 Hz, 1H), 5.72 (d, J=5.5 Hz, 1H), 4.49-4.61 (m, 1H), 4.36 (d, J=11.7 Hz, 1H), 4.04 (d, J=11.7 Hz, 1H), 3.58 (s, 1H), 2.93 (q, J=6.4 Hz, 2H), 2.69-2.79 (m, 1H), 2.57-2.40 (m, 1H, overlapping DMSO peak), 1.08-1.44 (m, 20H), 0.78-0.91 (m, 3H).

### Example 4: ((2R,3S,5R)-5-(6-amino-2-fluoro-9H-purin-9-yl)-2-ethynyl-3-hydroxytetrahydrofuran-2-yl)methyl pentylcarbamate

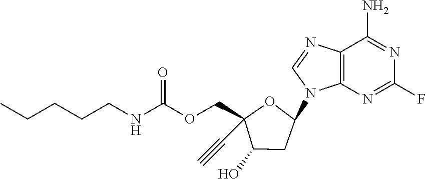 [SMILES: C#C[C@]1(COC(=O)NCCCCC)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting pentan-1-amine for octadecan-1-amine is step A. LCMS (ESI) m/z calcd for C₁₈H₂₃FN₆O₄: 406.2. Found: 407.3 (M+1)⁺. ¹H NMR (400 MHz, DMSO-d₆) δ 8.25 (s, 1H), 7.81 (br s, 2H), 7.20-7.15 (m, 1H), 6.24 (dd, J=4.9, 7.5 Hz, 1H), 5.71 (d, J=5.5 Hz, 1H), 4.60-4.49 (m, 1H), 4.35 (d, J=11.7 Hz, 1H), 4.03 (d, J=11.4 Hz, 1H), 3.57 (s, 1H), 2.96-2.89 (m, 2H), 2.81-2.70 (m, 1H), 2.53-2.41 (m, 1H, overlapping DMSO peak), 1.43-1.09 (m, 6H), 0.83 (t, J=7.0 Hz, 3H).

### Example 5: ((2R,3S,5R)-5-(6-amino-2-fluoro-9H-purin-9-yl)-2-ethynyl-3-hydroxytetrahydrofuran-2-yl)methyl methylcarbamate

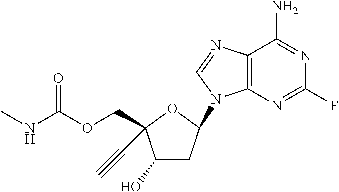 [SMILES: C#C[C@]1(COC(=O)NC)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting 40% aqueous methylamine for octadecan-1-amine is step A. LCMS (ESI) m/z calcd for C₁₄H₁₅FN₆O₄: 350.1. Found: 351.2 (M+1)⁺. ¹H NMR (400 MHz, METHANOL-d₄) δ 8.12 (s, 1H), 6.32 (dd, J=4.3, 7.6 Hz, 1H), 4.77-4.72 (m, 1H), 4.46 (d, J=11.7 Hz, 1H), 4.23 (d, J=11.7 Hz, 1H), 3.14 (s, 1H), 2.86-2.78 (m, 1H), 2.71-2.57 (m, 4H).

### Example 6: ((2R,3S,5R)-5-(6-amino-2-fluoro-9H-purin-9-yl)-2-ethynyl-3-hydroxytetrahydrofuran-2-yl)methyl isopropylcarbamate

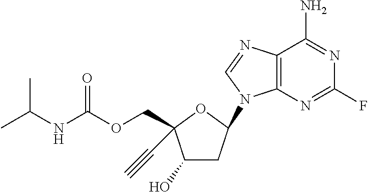 [SMILES: C#C[C@]1(COC(=O)NC(C)C)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting isopropylamine for octadecan-1-amine is step A. LCMS (ESI) m/z calcd for C₁₆H₁₉FN₆O₄: 378.2. Found: 379.2 (M+1)⁺. ¹H NMR (400 MHz, DMSO-d₆) δ 8.25 (s, 1H), 7.81 (br s, 2H), 7.12-7.02 (m, 1H), 6.24 (dd, J=4.9, 7.5 Hz, 1H), 5.70 (d, J=5.2 Hz, 1H), 4.61-4.51 (m, 1H), 4.39-4.29 (m, 1H), 4.05-3.94 (m, 1H), 3.65-3.45 (m, 2H), 2.81-2.70 (m, 1H), 2.57-2.40 (m, 1H, overlapping DMSO peak), 1.10-0.93 (m, 6H).

### Example 7: ((2R,3S,5R)-5-(6-amino-2-fluoro-9H-purin-9-yl)-2-ethynyl-3-hydroxytetrahydrofuran-2-yl)methyl diethylcarbamate

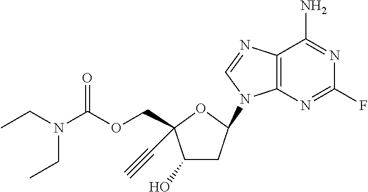 [SMILES: C#C[C@]1(COC(=O)N(CC)CC)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting diethylamine for octadecan-1-amine is step A. LCMS (ESI) m/z calcd for C₁₇H₂₁FN₆O₄: 392.2. Found: 393.2 (M+1)⁺. ¹H NMR (400 MHz, DMSO-d₆) δ 8.25 (s, 1H), 7.80 (br s, 2H), 6.24 (dd, J=4.1, 8.1 Hz, 1H), 5.74 (d, J=5.5 Hz, 1H), 4.71-4.63 (m, 1H), 4.31 (d, J=11.4 Hz, 1H), 4.08 (d, J=11.7 Hz, 1H), 3.59 (s, 1H), 3.21-3.08 (m, 4H), 2.86-2.78 (m, 1H), 2.55-2.43 (m, 1H, overlapping DMSO peak), 0.96 (t, J=7.2 Hz, 6H).

### Example 8: ((2R,3S,5R)-5-(6-amino-2-fluoro-9H-purin-9-yl)-2-ethynyl-3-hydroxytetrahydrofuran-2-yl)methyl tert-butylcarbamate

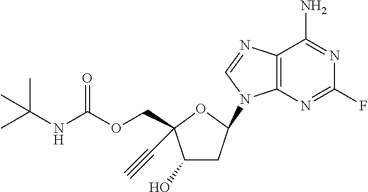 [SMILES: C#C[C@]1(COC(=O)NC(C)(C)C)O[C@@H](n2cnc3c(N)nc(F)nc32)C[C@@H]1O]

The title compound was prepared as described herein for the synthesis of ((2R,3S,5R)-5-(6-amino-2-fluoro-9H-purin-9-yl)-2-ethynyl-3-hydroxytetrahydrofuran-2-yl)methyl octadecylcarbamate, substituting t-butylamine for octadecan-1-amine is step A. LCMS (ESI) m/z calcd for C₁₇H₂₁FN₆O₄: 392.2. Found: 393.3 (M+1)⁺. ¹H NMR (400 MHz, DMSO-d₆) δ 8.27 (s, 1H), 7.82 (br s, 2H), 6.88-6.80 (m, 1H), 6.25 (dd, J=5.0, 7.6 Hz, 1H), 5.69 (d, J=5.2 Hz, 1H), 4.65-4.54 (m, 1H), 4.35 (d, J=11.7 Hz, 1H), 4.01-3.92 (m, 1H), 3.59 (s, 1H), 2.83-2.70 (m, 1H), 2.54-2.41 (m, 1H, overlapping DMSO peak), 1.18 (s, 9H).

### Example 9: 2-(((((2R,3S,5R)-5-(6-amino-2-fluoro-9H-purin-9-yl)-2-ethynyl-3-hydroxytetrahydrofuran-2-yl)methoxy)carbonyl)oxy)propane-1,3-diyl dinonanoate

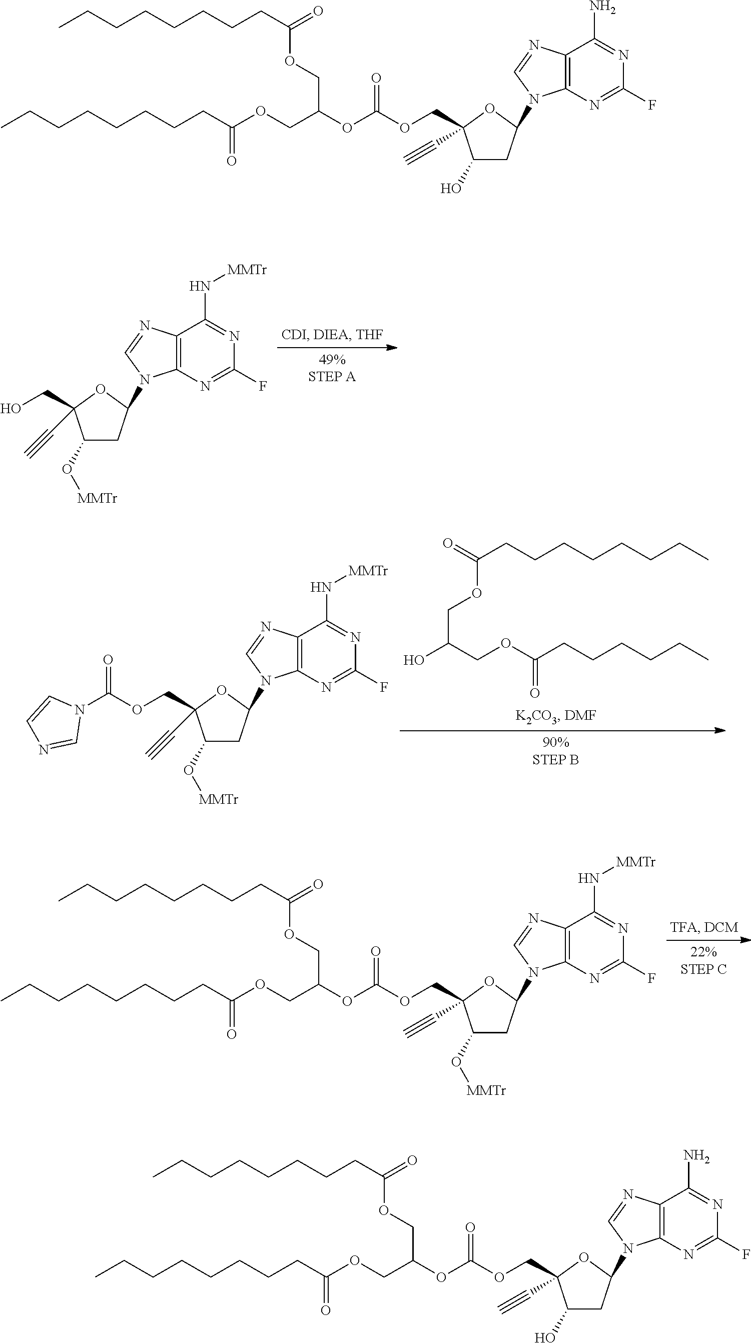

Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate. To a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (300 mg, 0.358 mmol), in THF (4 mL) stirred at RT was added DIEA (0.125 mL, 0.716 mmol) and CDI (116 mg, 0.716 mmol). The reaction mixture was stirred at RT for overnight. LCMS indicated complete reaction. The solvent was removed in vacuo. The residue was subjected to preparative TLC (silica gel, 30:1 DCM/MeOH) to give the desired product (170 mg, 48%) as a white solid. LCMS (ESI) m/z calcd for C₅₆H₄₆FN₇O₆: 931. Found: 932 (M+1)⁺. 1 H NMR (300 MHz, CDCl₃) δ 7.90 (s, 1H), 7.69 (s, 2H), 7.53 (t, J=15.0 Hz, 4H), 7.40 (d, J=9.0 Hz, 2H), 7.29-7.19 (m, 10H), 7.15-7.10 (m, 8H), 7.01 (s, 1H), 6.88 (s, 1H), 6.77 (t, J=15.0 Hz, 4H), 6.07-6.03 (m, 1H), 4.90 (t, J=18.0 Hz, 1H), 4.45 (t, J=12.0 Hz, 1H), 4.24 (t, J=12.0 Hz, 1H), 3.78 (s, 3H), 3.68 (s, 3H), 2.86 (s, 1H), 2.46-2.36 (m, 1H), 2.11-2.06 (m, 1H).

Step B: 2-(((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)carbonyl)oxy)propane-1,3-diyl dinonanoate. To a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate (130 mg, 0.139 mmol), in DMF (1 mL) stirred at RT was added K₂CO₃ (38.6 mg, 0.279 mmol) followed by 2-hydroxypropane-1,3-diyl dinonanoate (312 mg, 0.837 mmol, prepared as described herein for the synthesis of 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dinonanoate, step B). The reaction mixture was stirred at RT for 3 h. LCMS indicated complete reaction. The mixture was diluted with EtOAc (50 mL), washed with brine, dried over Na₂SO₄ and concentrated to dryness at reduced pressure. The residue was subjected to preparative TLC (silica gel, 50:1 DCM/MeOH) to give the desired product (200 mg, 90%) as a white solid. LCMS (ESI) m/z calcd for C₇₄H₈₂FN₅O₁₁: 1235. Found: 1236 (M+1)⁺. 1H NMR (300 MHz, DMSO-d₆) δ 8.25 (s, 1H), 7.91 (s, 1H), 7.50 (d, J=6.0 Hz, 4H), 7.37-7.17 (m, 20H), 6.86-6.82 (m, 4H), 6.15 (t, J=9.0 Hz, 1H), 4.96 (t, J=12.0 Hz, 1H), 4.64 (t, J=15.0 Hz, 1H), 4.28-4.10 (m, 1H), 4.06-3.98 (m, 1H), 3.94 (s, 1H), 3.82 (d, J=12.0 Hz, 1H), 3.69 (d, J=9.0 Hz, 6H), 3.48 (t, J=9.0 Hz, 1H), 2.31-2.16 (m, 4H), 1.51-1.40 (m, 4H), 1.20 (m, 24H), 0.87-0.78 (m, 6H).

Step C: 2-(((((2R,3S,5R)-5-(6-amino-2-fluoro-9H-purin-9-yl)-2-ethynyl-3-hydroxytetrahydrofuran-2-yl)methoxy)carbonyl)oxy) Propane-1,3-diyl dinonanoate. To a solution of 2-(((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)carbonyl)oxy)propane-1,3-diyl dinonanoate (45 mg, 0.036 mmol) in DCM (3 mL) was added TFA (0.308 mL, 4.00 mmol) and the resulting solution was stirred for 1 h at RT. LCMS indicated complete reaction. The solution was diluted with MeOH (3 mL) and concentrated to dryness at reduced pressure. The mixture was subjected to RP-HPLC purification (C18, MeCN/water, 0.1% formic acid) to give the desired product (5.6 mg, 22%) as a white solid. LCMS (ESI) m/z calcd for C₃₄H₅₀FN₅O₉: 691. Found: 692 (M+1)⁺. 1H NMR (400 MHz, METHANOL-d₄) δ 8.15 (s, 1H), 6.35-6.33 (m, 1H), 5.09-5.05 (m, 1H), 4.87-4.84 (m, 1H), 4.55 (d, J=12.0 Hz, 1H), 4.39-4.30 (m, 3H), 4.17-4.05 (m, 2H), 3.20 (s, 1H), 2.80-2.77 (m, 1H), 2.71-2.64 (m, 1H), 2.30 (m, 2H), 2.22 (t, J=16.0 Hz, 2H), 1.52 (t, J=12.0 Hz, 4H), 1.32-1.21 (m, 20H), 0.88 (t, J=12.0 Hz, 6H).

### Example 10: ((2R,3S,5R)-5-(6-amino-2-fluoro-9H-purin-9-yl)-2-ethynyl-3-hydroxytetrahydrofuran-2-yl)methyl (6,18-dioxo-1,5-dioxacyclooctadecan-3-yl) carbonate

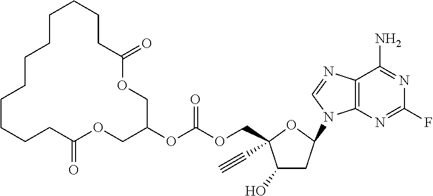 [SMILES: C#C[C@]1(COC(=O)OC2COC(=O)CCCCCCCCCCCC(=O)OC2)O[C@@H](n2cnc3c(N)nc(F)nc32)C[C@@H]1O]

The title compound was prepared as described herein for the synthesis of 2-(((((2R,3S,5R)-5-(6-amino-2-fluoro-9H-purin-9-yl)-2-ethynyl-3-hydroxytetrahydrofuran-2-yl)methoxy)carbonyl)oxy)propane-1,3-diyl dinonanoate, substituting 3-hydroxy-1,5-dioxacyclooctadecane-6,18-dione for 2-hydroxypropane-1,3-diyl dinonanoate in step B. LCMS (ESI) m/z calcd for C₂₉H₃₈FN₅O₉: 619. Found: 620 (M+1)⁺. 1H NMR (400 MHz, METHANOL-d₄)) δ 8.14 (s, 1H), 6.35-6.32 (m, 1H), 5.02-4.99 (m, 1H), 4.80 (t, J=12 Hz, 1H), 4.56 (d, J=12 Hz, 1H), 4.42-4.37 (m, 3H), 4.15-4.06 (m, 2H), 3.17 (s, 1H), 2.89-2.83 (m, 1H), 2.68-2.60 (m, 1H), 2.32-2.29 (m, 4H), 1.60-1.54 (m, 4H), 1.31-1.20 (m, 14H).

### Example 11: 4-((((((2R,3S,5R)-5-(6-amino-2-fluoro-9H-purin-9-yl)-2-ethynyl-2-(hydroxymethyl)tetrahydrofuran-3-yl)oxy)carbonyl)oxy)methyl)phenyl icosanoate

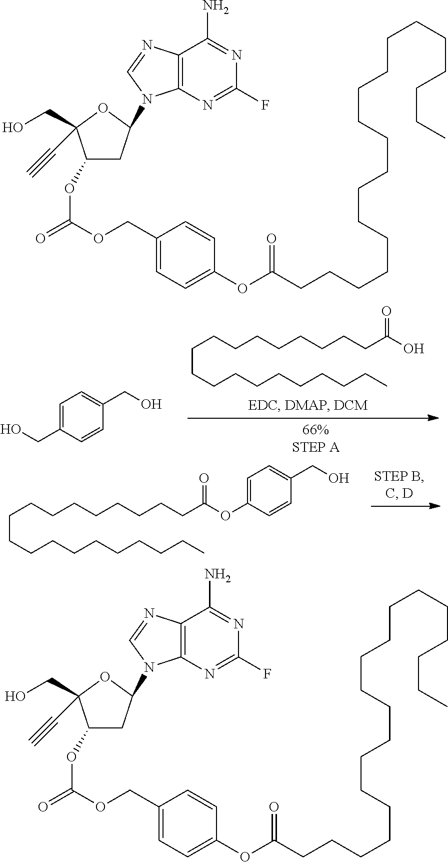

Step A: 4-(hydroxymethyl)phenyl icosanoate. A suspension of icosanoic acid (4.00 g, 12.8 mmol) in DCM (105 ml) was treated with 4-(hydroxymethyl)phenol (2.22 g, 17.9 mmol) and DMAP (2.19 g, 17.9 mmol). The mixture was cooled to 0° C., treated with EDC (3.43 g, 17.9 mmol), and then stirred over 18 h, letting the bath warm slowly to RT. The reaction was diluted with water and extracted with DCM. The combined organics were washed with saturated aqueous NaHCO₃, brine, dried over Na₂SO₄, filtered, and concentrated. Purification by flash chromatography (silica gel, 0-60% EtOAc/DCM) afforded 4-(hydroxymethyl)phenyl icosanoate (3.59 g, 65%) as white solid. ¹H NMR (400 MHz, CDCl3) δ 7.42-7.35 (m, 2H), 7.11-7.04 (m, 2H), 4.70 (d, J=6.0 Hz, 2H), 2.56 (t, J=7.5 Hz, 2H), 1.76 (quin, J=7.5 Hz, 2H), 1.61 (t, J=6.1 Hz, 1H), 1.46-1.23 (m, 32H), 0.93-0.86 (m, 3H).

Steps B, C, and D: 4-((((((2R,3S,5R)-5-(6-amino-2-fluoro-9H-purin-9-yl)-2-ethynyl-2-(hydroxymethyl)tetrahydrofuran-3-yl)oxy)carbon y)oxy)methyl)phenyl icosanoate. The title compound was prepared as described herein for the synthesis of 4-((((((2R,3S,5R)-5-(6-amino-2-fluoro-9H-purin-9-yl)-2-ethynyl-2-(hydroxymethyl)tetrahydrofuran-3-yl)oxy)carbonyl)oxy)methyl)phenyl tetradecanoate, steps B, C, and D. LCMS (ESI) m/z calcd for C₄₀H₅₆FN₅O₇: 737.4. Found: 738.5 (M+1)⁺. ¹H NMR (400 MHz, CDCl₃) δ 7.76 (s, 1H), 7.50-7.42 (m, 2H), 7.15-7.03 (m, 2H), 6.31 (dd, J=5.2, 9.8 Hz, 1H), 5.79 (br s, 2H), 5.67-5.57 (m, 2H), 5.30-5.24 (m, 1H), 5.19-5.11 (m, 1H), 4.07-3.88 (m, 2H), 3.25 (ddd, J=6.2, 9.8, 13.8 Hz, 1H), 2.60-2.46 (m, 3H), 2.39 (s, 1H), 1.82-1.69 (m, 2H), 1.51-1.20 (m, 32H), 0.95-0.84 (m, 3H).

### Example 12: 4-((((((2R,3S,5R)-5-(6-amino-2-fluoro-9H-purin-9-yl)-2-ethynyl-2-(hydroxymethyl)tetrahydrofuran-3-yl)oxy)carbonyl)oxy)methyl)phenyl tetradecanoate

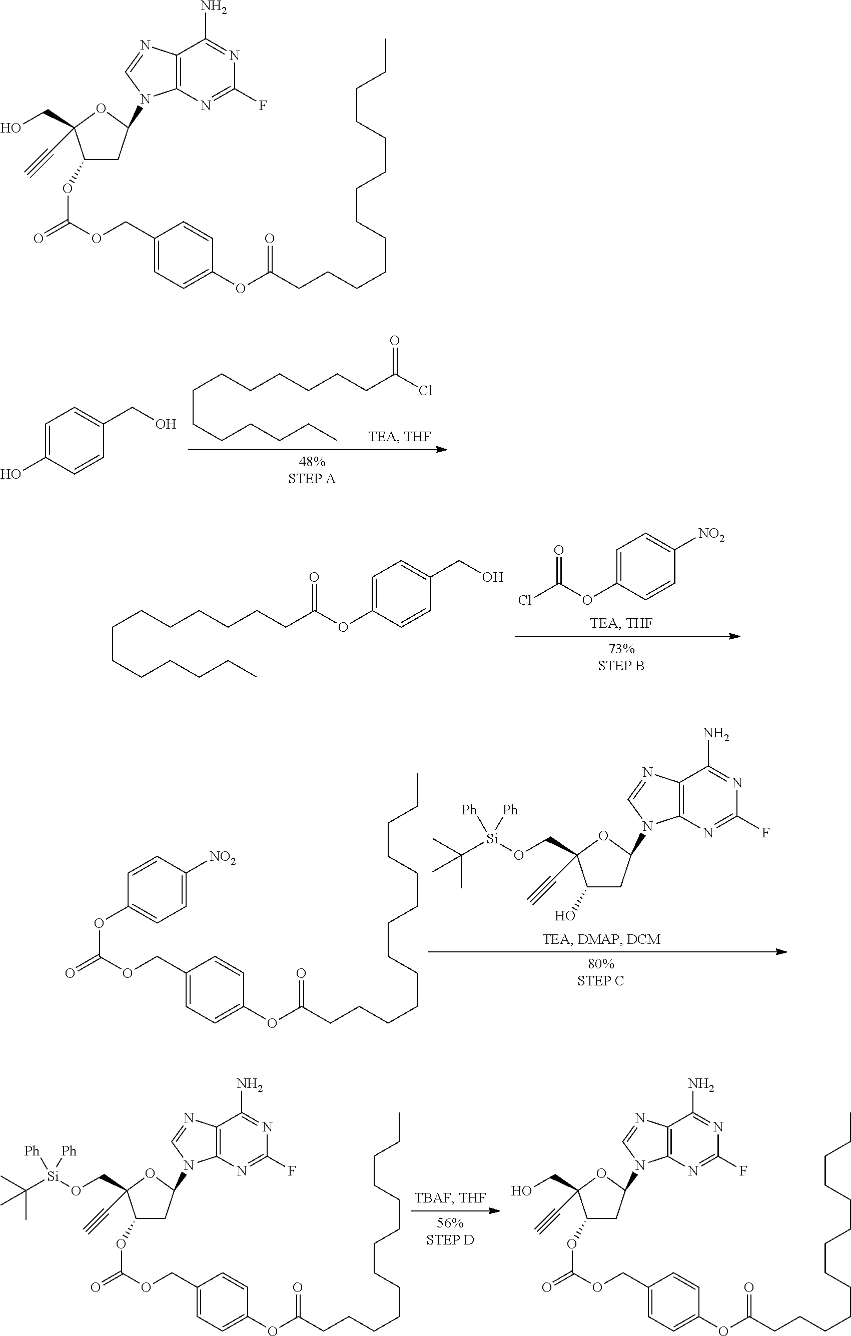

Step A: 4-(hydroxymethyl)phenyl tetradecanoate. An ice cold solution of 4-(hydroxymethyl)phenol (500 mg, 4.03 mmol) and TEA (0.561 mL, 4.03 mmol) in THF (7.5 mL) was treated dropwise with a solution of tetradecanoyl chloride (1.10 mL, 4.03 mmol) in THF (2.5 mL) over 25 min. The reaction was stirred at 0° C. for 3 h, and then warmed to RT for 18 h. The reaction was diluted with THF (2.5 mL), treated with additional TEA (190 uL), cooled to 0° C., and then treated with tetradecanoyl chloride (360 uL). The reaction was stirred at RT for 5.5 h, and the suspension was filtered. The filtrate was diluted with EtOAc, washed with saturated aqueous NaHCO₃, water, brine, dried over Na₂SO₄, filtered, and concentrated. Purification by flash chromatography (silica gel, 0-60% EtOAc/hexanes) afforded 4-(hydroxymethyl)phenyl tetradecanoate (665 mg, 48%) as white solid. ¹H NMR (400 MHz, CDCl₃) δ 7.42-7.34 (m, 2H), 7.11-7.04 (m, 2H), 4.70 (d, J=6.0 Hz, 2H), 2.56 (t, J=7.5 Hz, 2H), 1.76 (quin, J=7.5 Hz, 2H), 1.62 (t, J=6.0 Hz, 1H), 1.47-1.22 (m, 20H), 0.94-0.85 (m, 3H).

Step B: 4-((((4-nitrophenoxy)carbonyl)oxy)methyl)phenyl tetradecanoate. An ice cold solution of 4-(hydroxymethyl)phenyl tetradecanoate (500 mg, 1.50 mmol) and TEA (0.313 mL, 2.24 mmol) in THF (15 mL) was treated with 4-nitrophenyl carbonochloridate (362 mg, 1.79 mmol), stirred at 0° C. for 10 min, and then at RT for 3.5 h. The reaction was diluted with EtOAc, washed with 1N HCl, saturated aqueous NaHCO₃, brine, dried over Na₂SO₄, filtered, and concentrated. Purification by flash chromatography (silica gel, 0-40% EtOAc/hexanes) afforded 4-((((4-nitrophenoxy)carbonyl)oxy)methyl)phenyl tetradecanoate (553 mg, 73%) as white solid. ¹H NMR (400 MHz, CDCl₃) δ 8.33-8.25 (m, 2H), 7.51-7.44 (m, 2H), 7.42-7.36 (m, 2H), 7.17-7.10 (m, 2H), 5.29 (s, 2H), 2.57 (t, J=7.5 Hz, 2H), 1.77 (quin, J=7.5 Hz, 2H), 1.47-1.22 (m, 20H), 0.95-0.85 (m, 3H).

Step C: 4-((((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)oxy)methyl)phenyl tetradecanoate. A mixture of (2R,3S,5R)-5-(6-amino-2-fluoro-9H-purin-9-yl)-2-(((tert-butyldiphenylsilyl)oxy)methyl)-2-ethynyltetrahydrofuran-3-ol (150 mg, 0.282 mmol) and 4-((((4-nitrophenoxy)carbonyl)oxy)methyl)phenyl tetradecanoate (282 mg, 0.564 mmol) in DCM (2821 μl) was treated with DMAP (34.5 mg, 0.282 mmol), TEA (118 μl, 0.846 mmol), and stirred at RT for 18 h. The reaction was diluted with saturated aqueous NaHCO₃, extracted with DCM, washed with saturated aqueous NaHCO₃, brine, dried over Na₂SO₄, filtered, and concentrated. Purification by flash chromatography (silica gel, 0-100% EtOAc/hexanes) afforded 4-((((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)oxy)methyl)phenyl tetradecanoate (201 mg, 80%) as clear film. LCMS (ESI) m/z calcd for C₅₀H₆₂FN₅O₇Si: 891.4. Found: 892.5 (M+1)⁺. ¹H NMR (400 MHz, CDCl₃) δ 7.91 (s, 1H), 7.70-7.62 (m, 4H), 7.49-7.32 (m, 8H), 7.13-7.06 (m, 2H), 6.45 (dd, J=6.1, 7.5 Hz, 1H), 5.75-5.59 (m, 3H), 5.29-5.22 (m, 1H), 5.21-5.13 (m, 1H), 4.09-4.02 (m, 1H), 3.99-3.92 (m, 1H), 2.97 (td, J=7.1, 14.1 Hz, 1H), 2.74 (ddd, J=3.7, 6.1, 13.8 Hz, 1H), 2.56 (t, J=7.5 Hz, 2H), 2.43 (s, 1H), 1.83-1.70 (m, 2H), 1.48-1.21 (m, 20H), 1.09 (s, 9H), 0.94-0.85 (m, 3H).

Step D: 4-((((((2R,3S,5R)-5-(6-amino-2-fluoro-9H-purin-9-yl)-2-ethynyl-2-(hydroxymethyl)tetrahydrofuran-3-yl)oxy)carbonyl)oxy)methyl)phenyl tetradecanoate. An ice cold solution of 4-((((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)oxy)methyl)phenyl tetradecanoate (265 mg, 0.297 mmol) in THF (6 mL) was treated with TBAF (1 M in THF) (0.446 mL, 0.446 mmol), and stirred at 0° C. for 1.5 h. The reaction was quenched with AcOH (˜0.5 mL), diluted with water, and extracted with EtOAc. The combined organics were washed with brine, dried over Na₂SO₄, filtered, and concentrated. Purification by flash chromatography (silica gel, 0-100% EtOAc/hexanes) afforded 4-((((((2R,3S,5R)-5-(6-amino-2-fluoro-9H-purin-9-yl)-2-ethynyl-2-(hydroxymethyl)tetrahydrofuran-3-yl)oxy)carbonyl)oxy)methyl)phenyl tetradecanoate (111 mg, 56%) as white solid. LCMS (ESI) m/z calcd for C₃₄H₄₄FN₅O₇: 653.3. Found: 654.4 (M+1)⁺. ¹H NMR (400 MHz, CDCl₃) δ 7.76 (s, 1H), 7.50-7.43 (m, 2H), 7.12-7.04 (m, 2H), 6.31 (dd, J=5.2, 9.8 Hz, 1H), 5.84 (br s, 2H), 5.67-5.58 (m, 2H), 5.31-5.24 (m, 1H), 5.20-5.11 (m, 1H), 4.06-3.86 (m, 2H), 3.25 (ddd, J=6.2, 9.7, 13.9 Hz, 1H), 2.62-2.47 (m, 3H), 2.40 (s, 1H), 1.75 (quin, J=7.5 Hz, 2H), 1.48-1.21 (m, 20H), 0.93-0.84 (m, 3H).

### Example 13: 4-((((((2R,3S,5R)-5-(6-amino-2-fluoro-9H-purin-9-yl)-2-ethynyl-3-hydroxytetrahydrofuran-2-yl)methoxy)carbonyl)oxy)methyl)phenyl tetradecanoate

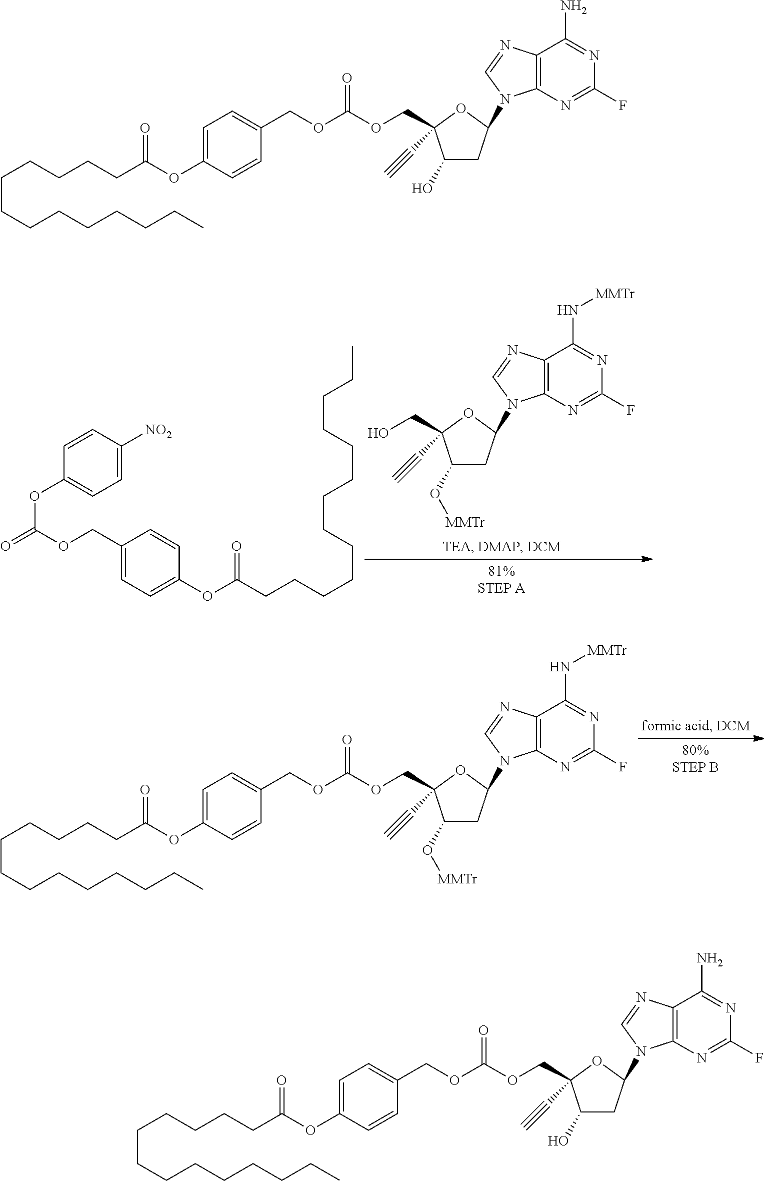

Step A: 4-((((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)carbonyl)oxy)methyl)phenyl tetradecanoate. A solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (350 mg, 0.418 mmol) and 4-((((4-nitrophenoxy)carbonyl)oxy)methyl)phenyl tetradecanoate (417 mg, 0.835 mmol) in DCM (4.2 mL) was treated with DMAP (51.0 mg, 0.418 mmol), TEA (0.175 mL, 1.25 mmol), and stirred at RT for 18 h. The reaction was diluted with saturated aqueous NaHCO₃, and extracted with DCM. The combined organics were washed with saturated aqueous NaHCO₃, brine, dried over Na₂SO₄, filtered, and concentrated. Purification by flash chromatography (silica gel, 0-70% EtOAc/hexanes) afforded 4-((((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)carbonyl)oxy)methyl)phenyl tetradecanoate (407 mg, 81%) as white solid. ¹H NMR (400 MHz, CDCl₃) δ 7.62 (s, 1H), 7.57-7.49 (m, 4H), 7.43-7.16 (m, 22H), 7.10-7.04 (m, 2H), 7.01 (s, 1H), 6.84-6.76 (m, 4H), 6.16 (dd, J=3.9, 7.7 Hz, 1H), 5.12-4.96 (m, 2H), 4.60 (t, J=7.3 Hz, 1H), 4.34 (d, J=11.7 Hz, 1H), 3.97 (d, J=11.7 Hz, 1H), 3.78 (s, 3H), 3.74 (s, 3H), 2.82 (s, 1H), 2.55 (t, J=7.5 Hz, 2H), 2.29-2.15 (m, 1H), 1.89-1.70 (m, 3H), 1.50-1.19 (m, 20H), 0.96-0.83 (m, 3H).

Step B: 4-((((((2R,3S,5R)-5-(6-amino-2-fluoro-9H-purin-9-yl)-2-ethynyl-3-hydroxytetrahydrofuran-2-yl)methoxy)carbonyl)oxy)methyl)phenyl tetradecanoate. A solution of 4-((((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)carbonyl)oxy)methyl)phenyl tetradecanoate (350 mg, 0.292 mmol) in DCM (6 mL) was treated with formic Acid (1.5 mL) and stirred at RT for 2 h. The reaction was quenched with MeOH and concentrated. Purification by flash chromatography (silica gel, 0-100% EtOAc/DCM) afforded 4-((((((2R,3S,5R)-5-(6-amino-2-fluoro-9H-purin-9-yl)-2-ethynyl-3-hydroxytetrahydrofuran-2-yl)methoxy)carbonyl)oxy)methyl)phenyl tetradecanoate (156 mg, 80%) as white solid. LCMS (ESI) m/z calcd for C₃₄H₄₄FN₅O₇: 653.3. Found: 654.5 (M+1)⁺. ¹H NMR (400 MHz, CDCl₃) δ 7.89 (s, 1H), 7.45-7.34 (m, 2H), 7.12-7.03 (m, 2H), 6.35 (dd, J=5.0, 7.2 Hz, 1H), 5.80 (br s, 2H), 5.20-5.08 (m, 2H), 4.78 (q, J=6.4 Hz, 1H), 4.59-4.39 (m, 2H), 2.96 (ddd, J=5.1, 6.5, 13.6 Hz, 1H), 2.78 (s, 1H), 2.69-2.46 (m, 4H), 1.75 (quin, J=7.5 Hz, 2H), 1.47-1.22 (m, 20H), 0.95-0.82 (m, 3H).

### Example 14: 4-((((((2R,3S,5R)-5-(6-amino-2-fluoro-9H-purin-9-yl)-2-ethynyl-3-hydroxytetrahydrofuran-2-yl)methoxy)carbonyl)oxy)methyl)phenyl icosanoate

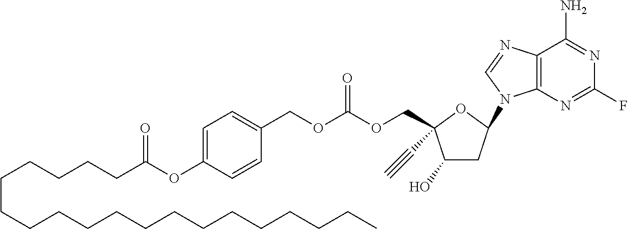 [SMILES: C#C[C@]1(COC(=O)OCc2ccc(OC(=O)CCCCCCCCCCCCCCCCCCC)cc2)O[C@@H](n2cnc3c(N)nc(F)nc32)C[C@@H]1O]

The title compound was prepared as described herein for the synthesis of 4-((((((2R,3S,5R)-5-(6-amino-2-fluoro-9H-purin-9-yl)-2-ethynyl-3-hydroxytetrahydrofuran-2-yl)methoxy)carbonyl)oxy)methyl)phenyl tetradecanoate, substituting 4-((((4-nitrophenoxy)carbonyl)oxy)methyl)phenyl icosanoate for 4-((((4-nitrophenoxy)carbonyl)oxy)methyl)phenyl tetradecanoate is step A. LCMS (ESI) m/z calcd for C₄₀H₅₆FN₅O₇: 737.4. Found: 738.6 (M+1)⁺. ¹H NMR (400 MHz, CDCl₃) δ 7.89 (s, 1H), 7.43-7.35 (m, 2H), 7.11-7.03 (m, 2H), 6.36 (dd, J=5.0, 7.2 Hz, 1H), 5.70 (br s, 2H), 5.19-5.09 (m, 2H), 4.78 (q, J=6.5 Hz, 1H), 4.59-4.52 (m, 1H), 4.49-4.42 (m, 1H), 2.96 (ddd, J=5.4, 6.4, 13.5 Hz, 1H), 2.78 (s, 1H), 2.69-2.50 (m, 3H), 2.38 (d, J=6.2 Hz, 1H), 1.83-1.70 (m, 2H), 1.48-1.19 (m, 32H), 0.92-0.84 (m, 3H).

### Example 15: (2R,3S,5R)-5-(6-amino-2-fluoro-9H-purin-9-yl)-2-ethynyl-2-(hydroxymethyl)tetrahydrofuran-3-yl dodecylcarbamate

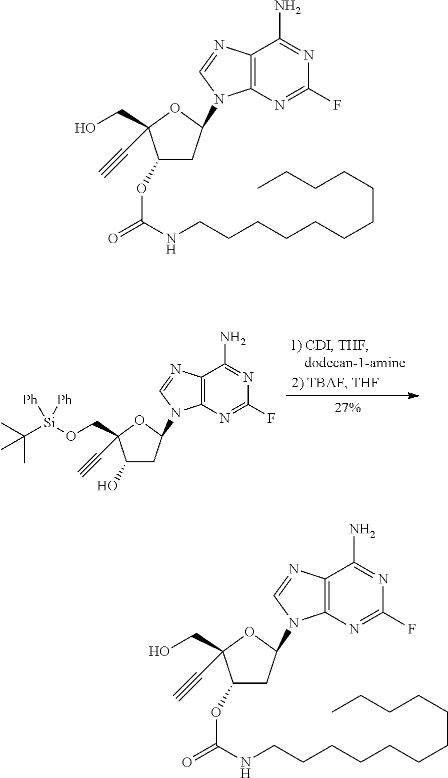 [SMILES: C#C[C@]1(CO)O[C@@H](n2cnc3c(N)nc(F)nc32)C[C@@H]1OC(=O)NCCCCCCCCCCCC ; C#C[C@]1(CO[Si](c2ccccc2)(c2ccccc2)C(C)(C)C)O[C@@H](n2cnc3c(N)nc(F)nc32)C[C@@H]1O ; C#C[C@]1(CO)O[C@@H](n2cnc3c(N)nc(F)nc32)C[C@@H]1OC(=O)NCCCCCCCCCCCC]

A solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (40 mg, 0.075 mmol) in THF 1.5 mL) was treated with CDI (18.3 mg, 0.113 mmol), and stirred at RT for 2 h. The reaction was then heated to 50° C. for 7.5 hours, and cooled to RT overnight. The reaction was treated with additional CDI (10.2 mg), stirred at 70° C. for 1.5 hours, treated with additional CDI (32 mg) and stirred at 70° C. for 1.5 hours until complete conversion of starting material to the active carbamate intermediate. The mixture was cooled to RT and treated with dodecan-1-amine (41.8 mg, 0.226 mmol). After 1.5 h the reaction was treated with an additional 97 mg portion of dodecan-1-amine and stirred at RT fo 4 days. The solution was concentrated at reduced pressure and the residue dissolved in THF (1.5 mL). The solution was cooled to O ° C. and treated with 1M TBAF/THF (0.113 mL, 0.113 mmol). After 1 h at 0° C. and 1 h at RT, the solution was quenched with AcOH (2 mL), diluted with water and extracted with EtOAc. The EtOAc solution was washed with brine, dried over Na₂SO₄, and concentrated to dryness at reduced pressure. Purification by flash chromatography (silica gel, 0-20% MeOH/EtOAc) followed by RP-HPLC (C18, MeCN/water with 0.1% formic acid) afforded the title compound (10.6 mg, 27%) as a white solid. LCMS (ESI) m/z calcd for C₂₅H₃₇FN₆O₄: 504.3. Found: 505.4 (M+1)⁺. ¹H NMR (400 MHz, METHANOL-d₄) δ 8.28 (s, 1H), 6.41 (t, J=6.9 Hz, 1H), 5.58 (br dd, J=3.9, 6.8 Hz, 1H), 3.92-3.79 (m, 2H), 3.12 (br t, J=6.9 Hz, 2H), 3.07 (s, 1H), 3.03-2.90 (m, 1H), 2.62 (ddd, J=4.1, 6.2, 13.8 Hz, 1H), 1.61-1.44 (m, 2H), 1.42-1.17 (m, 18H), 0.96-0.81 (m, 3H).

### Example 16: (2R,3S,5R)-5-(6-amino-2-fluoro-9H-purin-9-yl)-2-ethynyl-2-(hydroxymethyl)tetrahydrofuran-3-yl octadecylcarbamate

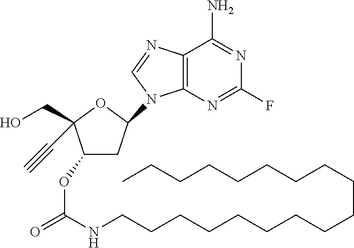 [SMILES: C#C[C@]1(CO)O[C@@H](n2cnc3c(N)nc(F)nc32)C[C@@H]1OC(=O)NCCCCCCCCCCCCCCCCCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting octadecan-1-amine for dodecan-1-amine. LCMS (ESI) m/z calcd for C₃₁H₄₉FN₆O₄: 588.4. Found: 589.5 (M+1)⁺. ¹H NMR (400 MHz, CDCl₃) δ 7.86 (s, 1H), 6.32 (dd, J=5.8, 8.5 Hz, 1H), 5.86 (br s, 2H), 5.74-5.61 (m, 1H), 5.18 (dd, J=3.8, 10.7 Hz, 1H), 4.93-4.74 (m, 1H), 4.08-3.85 (m, 2H), 3.32-3.04 (m, 3H), 2.62 (s, 1H), 2.58-2.45 (m, 1H), 1.57-1.48 (m, 2H), 1.45-1.06 (m, 30H), 0.96-0.82 (m, 3H).

### Example 17: (2R,3S,5R)-5-(6-amino-2-fluoro-9H-purin-9-yl)-2-ethynyl-2-(hydroxymethyl)tetrahydrofuran-3-yl octylcarbamate

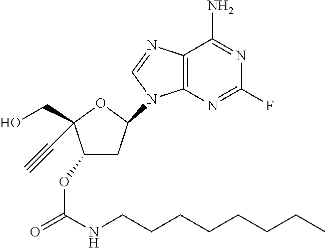 [SMILES: C#C[C@]1(CO)O[C@@H](n2cnc3c(N)nc(F)nc32)C[C@@H]1OC(=O)NCCCCCCCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting octan-1-amine for dodecan-1-amine. LCMS (ESI) m/z calcd for C₂₁H₂₉FN₆O₄: 448.2. Found: 449.3 (M+1)⁺. ¹H NMR (400 MHz, METHANOL-d₄) δ 8.28 (s, 1H), 7.12 (br t, J=5.6 Hz, 1H), 6.41 (t, J=6.8 Hz, 1H), 5.58 (dd, J=3.8, 6.7 Hz, 1H), 3.97-3.73 (m, 2H), 3.23-3.03 (m, 3H), 2.95 (td, J=7.1, 13.9 Hz, 1H), 2.62 (ddd, J=4.1, 6.3, 13.8 Hz, 1H), 1.64-1.44 (m, 2H), 1.43-1.21 (m, 10H), 0.98-0.78 (m, 3H).

### Example 18: (2R,3S,5R)-5-(6-amino-2-fluoro-9H-purin-9-yl)-2-ethynyl-2-(hydroxymethyl)tetrahydrofuran-3-yl methylcarbamate

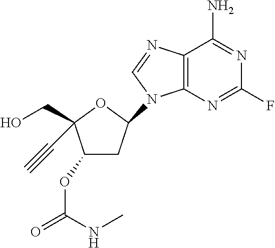 [SMILES: C#C[C@]1(CO)O[C@@H](n2cnc3c(N)nc(F)nc32)C[C@@H]1OC(=O)NC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting 40% aqueous methylamine for dodecan-1-amine. LCMS (ESI) m/z calcd for C₁₄H₁₅FN₆O₄: 350.1. Found: 351.2 (M+1)⁺. ¹H NMR (400 MHz, DMSO-d₆) δ 8.33 (s, 1H), 7.85 (br s, 2H), 7.33-7.15 (m, 1H), 6.28 (t, J=6.9 Hz, 1H), 5.56-5.39 (m, 2H), 3.77-3.51 (m, 3H), 2.92 (td, J=6.9, 14.0 Hz, 1H), 2.65-2.55 (m, 3H), 2.49-2.42 (m, 1H).

### Example 19: (2R,3S,5R)-5-(6-amino-2-fluoro-9H-purin-9-yl)-2-ethynyl-2-(hydroxymethyl)tetrahydrofuran-3-yl pentylcarbamate

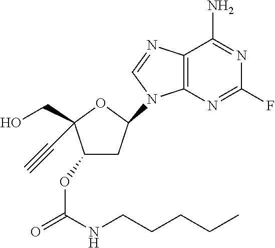 [SMILES: C#C[C@]1(CO)O[C@@H](n2cnc3c(N)nc(F)nc32)C[C@@H]1OC(=O)NCCCCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting pentan-1-amine for dodecan-1-amine. LCMS (ESI) m/z calcd for C₁₈H₂₃FN₆O₄: 406.2. Found: 407.3 (M+1)⁺. ¹H NMR (400 MHz, METHANOL-d₄) δ 8.28 (s, 1H), 6.41 (br t, J=6.7 Hz, 1H), 5.58 (br dd, J=3.7, 6.1 Hz, 1H), 3.96-3.73 (m, 2H), 3.24-3.03 (m, 3H), 3.02-2.85 (m, 1H), 2.72-2.52 (m, 1H), 1.62-1.44 (m, 2H), 1.44-1.22 (m, 4H), 1.05-0.78 (m, 3H).

### Example 20: (2R,3S,5R)-5-(6-amino-2-fluoro-9H-purin-9-yl)-2-ethynyl-2-(hydroxymethyl)tetrahydrofuran-3-yl isopropylcarbamate

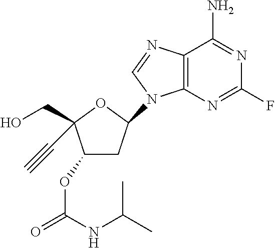 [SMILES: C#C[C@]1(CO)O[C@@H](n2cnc3c(N)nc(F)nc32)C[C@@H]1OC(=O)NC(C)C]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting isopropylamine for dodecan-1-amine. LCMS (ESI) m/z calcd for C₁₆H₁₉FN₆O₄: 378.2. Found: 379.3 (M+1)⁺. ¹H NMR (400 MHz, METHANOL-d₄) b 8.28 (s, 1H), 6.41 (t, J=6.9 Hz, 1H), 5.68-5.49 (m, 1H), 3.95-3.79 (m, 2H), 3.78-3.66 (m, 1H), 3.09 (s, 1H), 3.02-2.85 (m, 1H), 2.70-2.52 (m, 1H), 1.30-1.00 (m, 6H).

### Example 21: (2R,3S,5R)-5-(6-amino-2-fluoro-9H-purin-9-yl)-2-ethynyl-2-(hydroxymethyl)tetrahydrofuran-3-yl diethylcarbamate

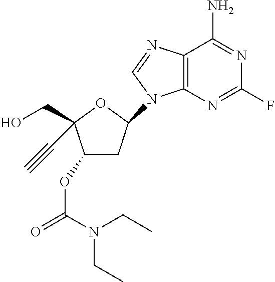 [SMILES: C#C[C@]1(CO)O[C@@H](n2cnc3c(N)nc(F)nc32)C[C@@H]1OC(=O)N(CC)CC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting diethylamine for dodecan-1-amine. LCMS (ESI) m/z calcd for C₁₇H₂₁FN₆O₄: 392.2. Found: 393.3 (M+1)⁺. ¹H NMR (400 MHz, METHANOL-d₄) δ 8.28 (s, 1H), 6.43 (t, J=6.8 Hz, 1H), 5.61 (dd, J=4.1, 6.9 Hz, 1H), 3.93-3.79 (m, 2H), 3.52-3.32 (m, 4H), 3.13 (s, 1H), 3.00 (td, J=7.1, 13.9 Hz, 1H), 2.66 (ddd, J=4.1, 6.4, 13.8 Hz, 1H), 1.30-1.07 (m, 6H).

### Example 22: (2R,3S,5R)-5-(6-amino-2-fluoro-9H-purin-9-yl)-2-ethynyl-2-(hydroxymethyl)tetrahydrofuran-3-yl 4-methylpiperidine-1-carboxylate

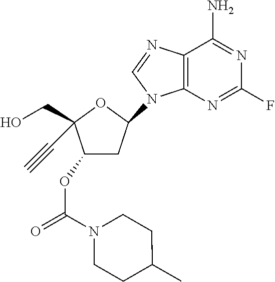 [SMILES: C#C[C@]1(CO)O[C@@H](n2cnc3c(N)nc(F)nc32)C[C@@H]1OC(=O)N1CCC(C)CC1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting 4-methylpiperidine for dodecan-1-amine. LCMS (ESI) m/z calcd for C₁₉H₂₃FN₆O₄: 418.2. Found: 419.3 (M+1)⁺. ¹H NMR (400 MHz, METHANOL-d₄) b 8.27 (s, 1H), 6.44 (dd, J=6.4, 7.4 Hz, 1H), 5.60 (dd, J=3.8, 6.7 Hz, 1H), 4.39-4.01 (m, 2H), 3.93-3.77 (m, 2H), 3.15 (br s, 1H), 3.05-2.73 (m, 3H), 2.70-2.55 (m, 1H), 1.79-1.51 (m, 3H), 1.27-1.06 (m, 2H), 0.97 (d, J=6.4 Hz, 3H).

### Example 23: (2R,3S,5R)-5-(6-amino-2-fluoro-9H-purin-9-yl)-2-ethynyl-2-(hydroxymethyl)tetrahydrofuran-3-yl tert-butylcarbamate

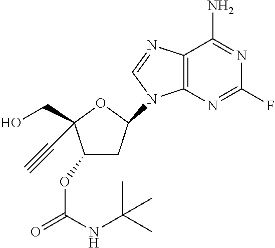 [SMILES: C#C[C@]1(CO)O[C@@H](n2cnc3c(N)nc(F)nc32)C[C@@H]1OC(=O)NC(C)(C)C]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting t-butylamine for dodecan-1-amine. LCMS (ESI) m/z calcd for C₁₇H₂₁FN₆O₄: 392.2. Found: 393.2 (M+1)⁺. ¹H NMR (400 MHz, METHANOL-d₄) δ 8.28 (s, 1H), 6.41 (t, J=6.9 Hz, 1H), 5.65-5.43 (m, 1H), 3.86 (s, 2H), 3.09 (s, 1H), 3.02-2.84 (m, 1H), 2.67-2.50 (m, 1H), 1.33 (s, 9H).

### Example 24: (2R,3S,5R)-5-(6-amino-2-fluoro-9H-purin-9-yl)-2-ethynyl-2-(hydroxymethyl)tetrahydrofuran-3-yl cyclopropylcarbamate

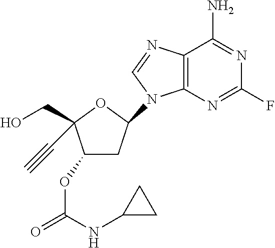 [SMILES: C#C[C@]1(CO)O[C@@H](n2cnc3c(N)nc(F)nc32)C[C@@H]1OC(=O)NC1CC1]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting cyclopropylamine for dodecan-1-amine. LCMS (ESI) m/z calcd for C₁₆H₁₇FN₆O₄: 376.1. Found: 377.2 (M+1)⁺. ¹H NMR (400 MHz, METHANOL-d₄) b 8.27 (s, 1H), 6.59-6.30 (m, 1H), 5.69-5.53 (m, 1H), 3.86 (s, 2H), 3.10 (s, 1H), 3.04-2.86 (m, 1H), 2.74-2.46 (m, 2H), 0.77-0.61 (m, 2H), 0.60-0.41 (m, 2H).

### Example 25: ethyl ((((2R,3S,5R)-5-(6-amino-2-fluoro-9H-purin-9-yl)-2-ethynyl-2-(hydroxymethyl)tetrahydrofuran-3-yl)oxy)carbonyl)glycinate

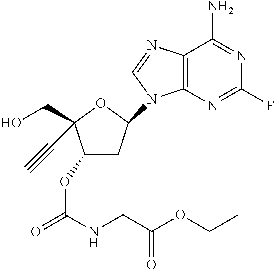 [SMILES: C#C[C@]1(CO)O[C@@H](n2cnc3c(N)nc(F)nc32)C[C@@H]1OC(=O)NCC(=O)OCC]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting ethyl glycinate hydrochloride for dodecan-1-amine. LCMS (ESI) m/z calcd for C₁₇H₁₉FN₆O₆: 422.1. Found: 423.2 (M+1)⁺. ¹H NMR (400 MHz, DMSO-d₆, 80° C.), δ 8.28 (s, 1H), 7.89-7.12 (m, 3H), 6.31 (t, J=6.9 Hz, 1H), 5.49 (dd, J=4.4, 6.8 Hz, 1H), 5.24 (t, J=6.2 Hz, 1H), 4.15 (q, J=7.2 Hz, 2H), 3.85-3.75 (m, 2H), 3.75-3.60 (m, 2H), 3.43 (s, 1H), 2.95 (td, J=6.9, 14.0 Hz, 1H), 2.57-2.52 (m, 1H), 1.23 (t, J=7.2 Hz, 3H).

### Example 26: tert-butyl ((((2R,3S,5R)-5-(6-amino-2-fluoro-9H-purin-9-yl)-2-ethynyl-2-(hydroxymethyl)tetrahydrofuran-3-yl)oxy)carbonyl)glycinate

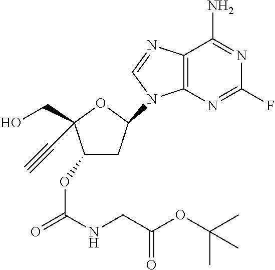 [SMILES: C#C[C@]1(CO)O[C@@H](n2cnc3c(N)nc(F)nc32)C[C@@H]1OC(=O)NCC(=O)OC(C)(C)C]

The title compound was prepared as described herein for the synthesis of (2R,3S,5R)-5-(6-amino-2-fluoro-9H-purin-9-yl)-2-ethynyl-2-(hydroxymethyl)tetrahydrofuran-3-yl dodecylcarbamate, substituting t-butylglycinate hydrochloride for dodecan-1-amine. LCMS (ESI) m/z calcd for C₁₉H₂₃FN₆O₆: 450.2. Found: 451.2 (M+1)⁺. ¹H NMR (400 MHz, DMSO-d₆, 80° C.) δ 8.28 (s, 1H), 7.75-7.47 (m, 2H), 7.46-7.23 (m, 1H), 6.31 (t, J=6.8 Hz, 1H), 5.49 (dd, J=4.4, 6.8 Hz, 1H), 3.79-3.59 (m, 4H), 3.43 (s, 1H), 2.94 (td, J=6.9, 13.8 Hz, 1H), 2.60-2.52 (m, 1H), 1.44 (s, 9H).

### Example 27: pentyl ((((2R,3S,5R)-5-(6-amino-2-fluoro-9H-purin-9-yl)-2-ethynyl-2-(hydroxymethyl)tetrahydrofuran-3-yl)oxy)carbonyl)glycinate

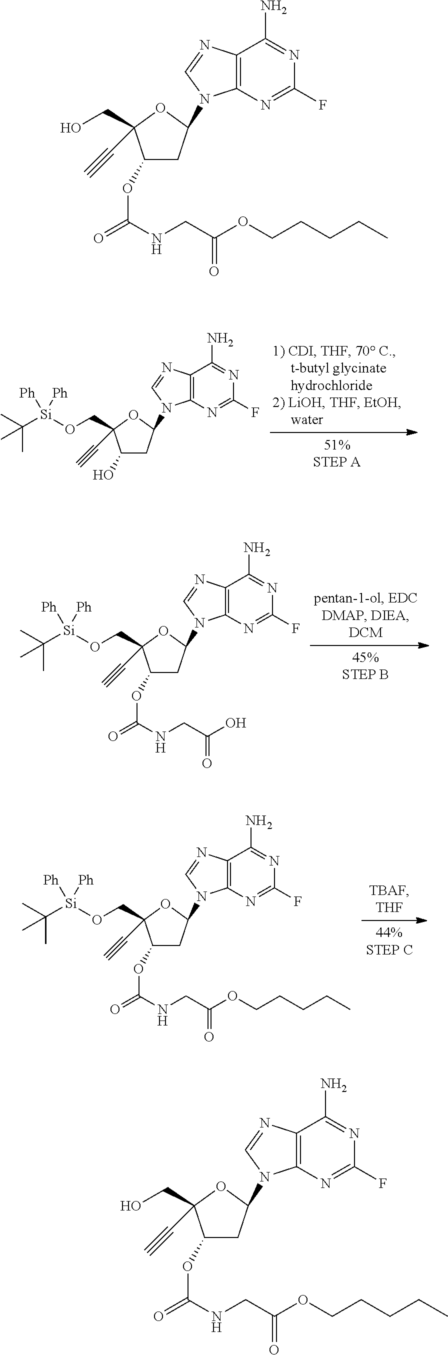

Step A: ((((2R,3S,5R)-5-(6-amino-2-fluoro-9H-purin-9-yl)-2-(((tert-butyldiphenylsilyl)oxymethyl)-2-ethynyltetrahydrofuran-3-yl)oxy)carbonyl)glycine. A solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (150 mg, 0.282 mmol) in THF (6 mL) was treated with CDI (91.0 mg, 0.564 mmol), and stirred at 70° C. for 1.5 h. The reaction mixture was treated with additional CDI (˜30 mg), and stirred at 70° C. for 1 h. The mixture was cooled slightly, treated with glycine tert-butyl ester hydrochloride (189 mg, 1.13 mmol), TEA (0.236 mL, 1.69 mmol), and stirred at RT for 4 days. The mixture was treated with additional glycine tert-butyl ester hydrochloride (189 mg, 1.13 mmol), TEA (0.236 mL, 1.69 mmol), THF (3 mL), and stirred at RT for an additional 18 h. The reaction was concentrated and purified by flash chromatography [silica gel, 0-75% (3:1 EtOAc:EtOH)/hexanes] to afford tert-butyl ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycinate as an impure, white solid. A portion (79 mg, 0.115 mmol) of this material was dissolved in THF (1.5 mL) and EtOH (0.5 mL), and the solution treated with 2M aqueous LiOH (0.287 mL, 0.573 mmol). After stirring at RT for 4.5 h the solution was treated with an additional portion (0.15 mL) of 2M LiOH. After an additional 30 minutes, the reaction mixture was diluted with saturated aqueous NH₄Cl, acidified with 1 N HCl to pH ˜6, and extracted with EtOAc. The combined organics were washed with brine, dried over Na₂SO₄, filtered, and concentrated to give crude ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycine (36.8 mg, 51%) as a clear film. The material was used without further purification. LCMS (ESI) m/z calcd for C₃₁H₃₃FN₆O₆Si: 632.2. Found: 633.3 (M+1)⁺. ¹H NMR (400 MHz, CDCl₃) δ 7.99 (s, 1H), 7.70-7.61 (m, 4H), 7.47-7.32 (m, 6H), 6.70-6.38 (m, 3H), 5.75 (dd, J=4.4, 6.6 Hz, 1H), 5.56 (br t, J=5.0 Hz, 1H), 4.10-3.88 (m, 4H), 2.92-2.81 (m, 1H), 2.78-2.68 (m, 1H), 2.58 (s, 1H), 1.08 (s, 9H).

Step B: pentyl ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycinate. A solution of ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycine (36 mg, 0.057 mmol) in DCM (1 mL) was treated with pentan-1-ol (0.012 mL, 0.114 mmol), DMAP (6.95 mg, 0.057 mmol), EDC (32.7 mg, 0.171 mmol), DIEA (0.050 mL, 0.284 mmol), and stirred at RT for 18 h. LCMS showed some remaining starting material. The reaction was treated with additional DCM (0.5 mL), pentan-1-ol (30 μl), DMAP (11 mg), EDC (38 mg), DIEA (0.1 mL), and stirred at RT for 5.5 h. The reaction was concentrated and purified by flash chromatography (silica gel, 0-100% EtOAc/hexanes) to give pentyl ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycinate (18 mg, 45%) as clear film. LCMS (ESI) m/z calcd for C₃₆H₄₃FN₆O₆Si: 702.3. Found: 703.4 (M+1)⁺. ¹H NMR (400 MHz, CDCl₃) δ 7.95 (s, 1H), 7.72-7.62 (m, 4H), 7.47-7.33 (m, 6H), 6.47 (t, J=6.7 Hz, 1H), 5.90-5.70 (m, 3H), 5.37 (br t, J=5.1 Hz, 1H), 4.19 (t, J=6.7 Hz, 2H), 4.08-3.91 (m, 4H), 2.92-2.81 (m, 1H), 2.71 (ddd, J=4.3, 6.2, 13.8 Hz, 1H), 2.59 (s, 1H), 1.73-1.64 (m, 2H), 1.40-1.32 (m, 4H), 1.09 (s, 9H), 0.95-0.88 (m, 3H).

Step C: pentyl ((((2R,3S,5R)-5-(6-amino-2-fluoro-9H-purin-9-yl)-2-ethynyl-2-(hydroxymethyl)tetrahydrofuran-3-yl)oxy)carbonyl)glycinate. An ice cold solution of pentyl ((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)glycinate (32 mg, 0.046 mmol) in THF (1 mL) was treated with TBAF (1 M in THF) (0.068 mL, 0.068 mmol) and stirred at 0° C. for 40 min. The reaction was quenched with AcOH (˜0.5 mL), diluted with water, and extracted with EtOAc. The combined organics were washed with brine 4×, dried over Na₂SO₄, filtered, and concentrated. Purification by flash chromatography (silica gel, 0-20% MeOH/DCM) followed by RP-HPLC (C18, MeCN/water, 0.1% formic acid) afforded the title compound (9.8 mg, 44%) as a white solid. LCMS (ESI) m/z calcd for C₃₆H₄₃FN₆O₆Si: 464.2. Found: 463.3 (M+1)⁺. ¹H NMR (400 MHz, DMSO-d₆, 80° C.) b 8.28 (s, 1H), 7.63-7.35 (m, 2H), 6.31 (t, J=6.8 Hz, 1H), 5.49 (dd, J=4.3, 6.7 Hz, 1H), 5.24 (t, J=6.1 Hz, 1H), 4.09 (t, J=6.6 Hz, 2H), 3.86-3.75 (m, 2H), 3.75-3.59 (m, 2H), 3.42 (s, 1H), 2.94 (td, J=6.9, 13.8 Hz, 1H), 2.60-2.52 (m, 1H), 1.70-1.53 (m, 2H), 1.40-1.25 (m, 4H), 0.93-0.82 (m, 3H).

### Example 28: 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl diacetate

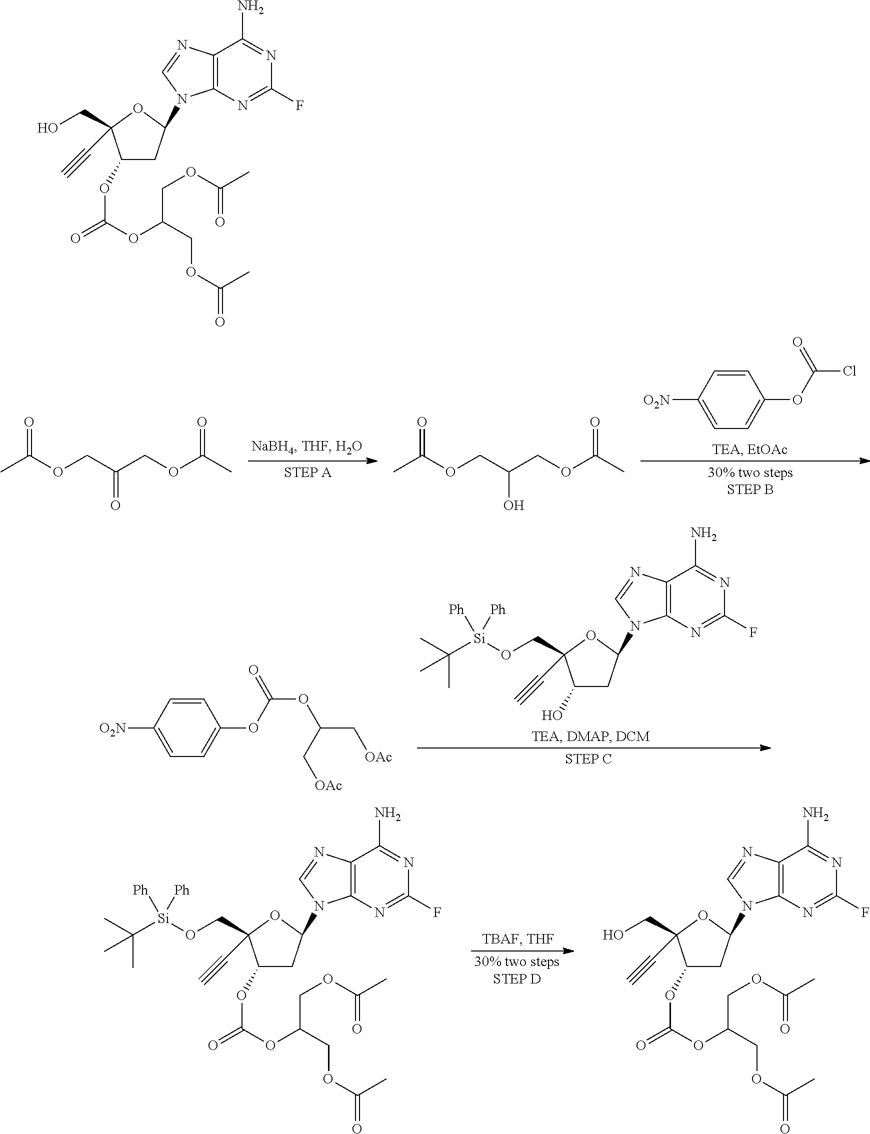

Step A: 2-hydroxypropane-1,3-diyl diacetate. A mixture of 2-oxopropane-1,3-diyl diacetate (100 mg, 0.574 mmol) in THF (2.0 mL) and water (0.1 mL) was treated at 0° C. with NaBH₄ (21.7 mg, 0.574 mmol) and the mixture was stirred for 30 min. Excess borohydride was destroyed by dropwise addition of glacial acetic acid (0.01 mL), the solution was diluted with DCM, washed with water, aqueous NaHCO₃ and dried over sodium sulfate. Concentrated and used in the next step without further purification.

Step B: 2-(((4-nitrophenoxy)carbonyl)oxy)propane-1,3-diyl diacetate. A mixture of 2-hydroxypropane-1,3-diyl diacetate and TEA (0.096 mL, 0.69 mmol) in EtOAc (3.0 mL) was treated with 4-nitrophenyl carbonochloridate (116 mg, 0.574 mmol) (a white ppt formed) and the mixture was stirred at RT for 3 h (solution turned bright yellow). The reaction was diluted with EtOAc, the precipitate removed by filtration and the filtrate concentrated. Purification by preparative TLC (DCM/MeOH, 4%) afforded the desired product as colorless oil.

Step C: 2-(((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)oxy)propane-1,3-diyl diacetate. A mixture of (2R,3S,5R)-5-(6-amino-2-fluoro-4,5-dihydro-9H-purin-9-yl)-2-(((tert-butyldiphenylsilyl)oxy)methyl)-2-ethynyltetrahydrofuran-3-ol (33 mg, 0.062 mmol) and 2-(((4-nitrophenoxy)carbonyl)oxy)propane-1,3-diyl diacetate (25.3 mg, 0.074 mmol) in DCM (2.0 mL) was treated with TEA (0.017 mL, 0.12 mmol) followed by DMAP (7.6 mg, 0.062 mmol) and the mixture was stirred at RT for 16 h. LCMS showed desired product forming. Concentrated and purified by preparative TLC (DCM/MeOH, 4%) to give the desired product which was impure and was used in the next step without further purification.

Step D: 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl diacetate. To a solution of 2-(((((2R,3S,5R)-5-(6-amino-2-fluoro-9H-purin-9-yl)-2-(((tert-butyldiphenylsilyl)oxy)methyl)-2-ethynyltetrahydrofuran-3-yl)oxy)carbonyl)oxy)propane-1,3-diyl diacetate in THF (4.0 mL) was added TBAF (1M, THF) (0.093 mL, 0.093 mmol) and the mixture was stirred at RT for 1.5 h. The mixture was concentrated and the residue purified by RP-HPLC (C18, MeCN/water, 0.1% formic acid) to give the desired product as a white solid (30%, two steps). LCMS (ESI) m/z calcd for C₂₀H₂₂FN₅O₉: 495. Found: 496 (M+1)⁺. ¹H NMR (400 MHz, METHANOL-d₄) δ 8.27 (d, J=1.4 Hz, 1H), 6.45 (dd, J=7.9, 6.2 Hz, 1H), 5.58-5.64 (m, 1H), 5.26-5.34 (m, 1H), 5.14-5.19 (m, 1H), 4.80 (s, 11H), 4.20-4.49 (m, 4H), 3.83-3.95 (m, 2H), 3.05-3.22 (m, 1H), 2.66-2.79 (m, 1H), 2.04-2.14 (m, 6H).

### Example 29: 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl dipentanoate

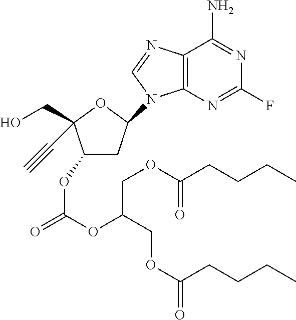 [SMILES: C#C[C@]1(CO)O[C@@H](n2cnc3c(N)nc(F)nc32)C[C@@H]1OC(=O)OC(COC(=O)CCCC)COC(=O)CCCC]

The title compound was prepared as described herein for the synthesis of 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl diacetate, substituting 2-hydroxypropane-1,3-diyl dipentanoate for 2-hydroxypropane-1,3-diyl diacetate is step B. LCMS (ESI) m/z calcd for C₂₆H₃₄FN₅O₉: 579. Found: 580 (M+1)⁺. ¹H NMR (400 MHz, CDCl₃) δ 7.83 (s, 1H), 6.89 (br s, 2H), 6.37 (dd, J=9.2, 5.6 Hz, 1H), 5.66 (dd, J=6.2, 1.4 Hz, 1H), 5.11-5.19 (m, 1H), 4.40 (ddd, J=14.3, 12.2, 4.3 Hz, 2H), 4.24 (ddd, J=12.3, 6.6, 5.7 Hz, 2H), 4.05 (d, J=12.6 Hz, 1H), 3.95 (br d, J=12.6 Hz, 1H), 3.22 (ddd, J=14.1, 9.2, 6.3 Hz, 1H), 2.70 (s, 1H), 2.60 (ddd, J=14.0, 5.6, 1.4 Hz, 1H), 2.35 (td, J=7.5, 4.3 Hz, 4H), 1.57-1.67 (m, 4H), 1.29-1.43 (m, 4H), 0.92 (br t, J=7.3 Hz, 3H), 0.91 (br t, J=7.3 Hz, 3H).

### Example 30: 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl dinonanoate

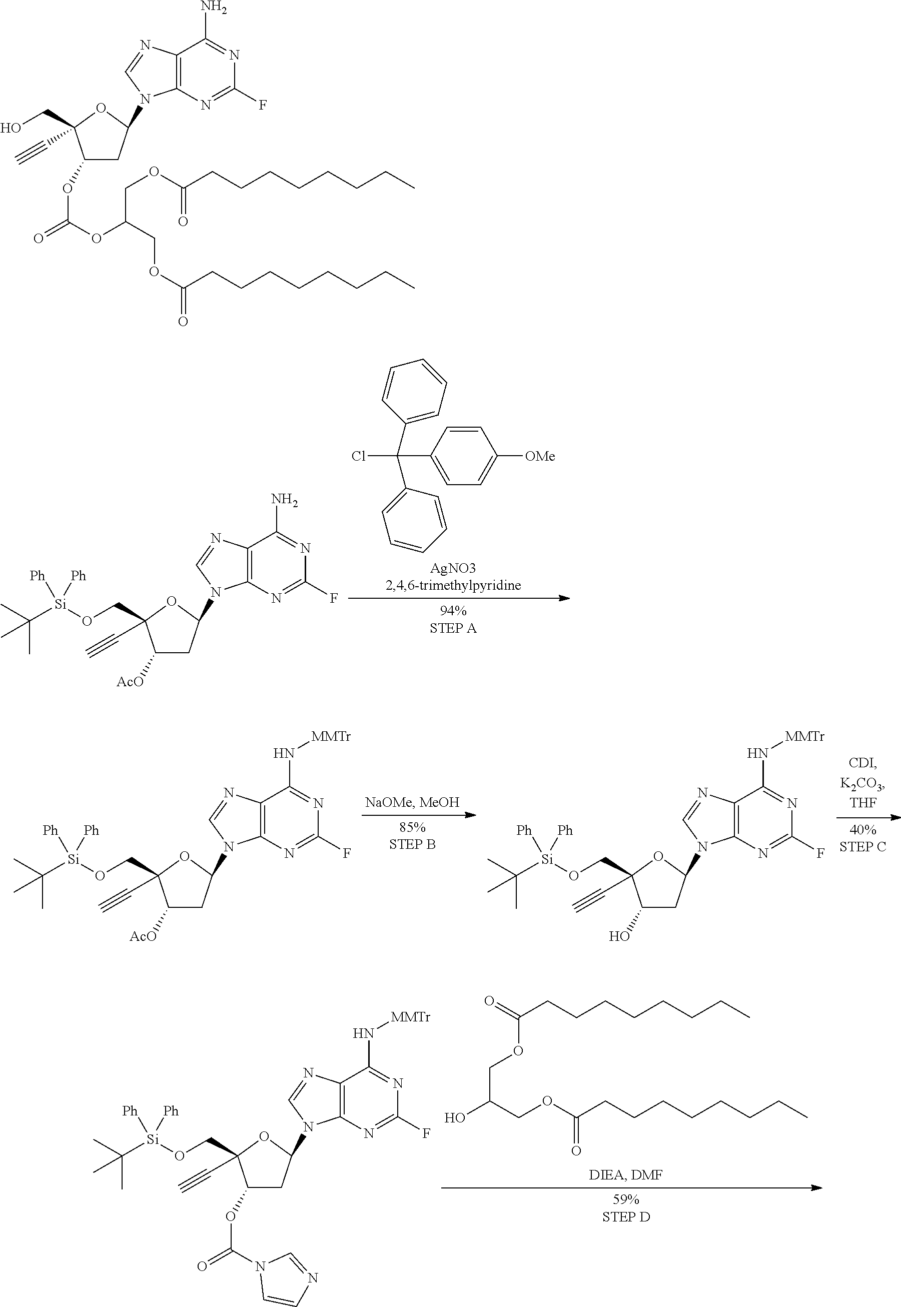

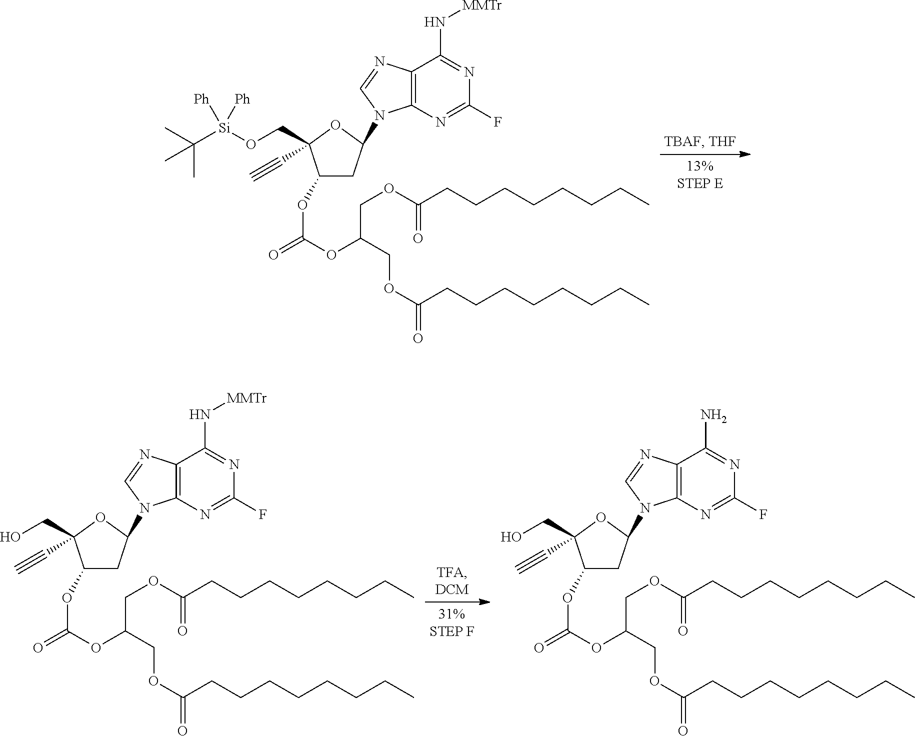

Step A: (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl acetate. (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (5.00 g, 8.72 mmol) was dissolved in DCM (100 mL). To the resulting solution was added 2,4,6-trimethylpyridine (23.0 mL, 174 mmol), silver(I)nitrate (14.8 g, 87.0 mmol) and (chloro(4-methoxyphenyl)methylene)dibenzene (26.9 g, 87.0 mmol). The mixture was stirred for 30 min at RT. The reaction mixture was filtered and the solid rinsed with DCM. The filtrate was mixed with 10% aqueous citric acid and the phases separated. The aqueous phase was extracted with DCM. The combined DCM solutions were washed with saturated aqueous NaHCO₃, dried over Na₂SO₄, and concentrated to dryness at reduced pressure. The crude residue was subjected to flash chromatography (silica gel, 1:1 EtOAc/petroleum ether) followed by preparative RP-HPLC (C18, MeCN/water, 0.1% formic acid) to afford the title compound (7.0 g, 94%) as a white solid. LCMS (ESI) m/z calcd for C₅₀H₄₈FN₅O₅Si: 845. Found: 846 (M+1)⁺.

Step B: (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-ol. (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl acetate (600 mg, 0.709 mmol) was dissolved in THF (2.5 mL)/MeOH (2.5 mL). Sodium methoxide (77 mg, 0.426 mmol) was added, and the resulting mixture was stirred for 10 min at RT. The reaction mixture was quenched with AcOH (1 mL), and concentrated under vacuum. The residue was purified by flash chromatography (silica gel, 50:1 DCM/MeOH) to give the desired product (500 mg, 85%) as a white solid. LCMS (ESI) m/z calcd for C₄₈H₄₆FN₅O₄Si: 803. Found: 804 (M+1)⁺. ¹H NMR (400 MHz, CDCl₃) δ 7.90 (s, 1H), 7.73-7.61 (m, 4H), 7.47-7.29 (m, 13H), 7.28-7.22 (m, 5H), 6.87-6.77 (m, 2H), 6.35 (dd, J=7.1, 5.3 Hz, 1H), 4.84 (t, J=6.4 Hz, 1H), 4.08-3.84 (m, 2H), 3.80 (s, 3H), 2.92 (dt, J=13.5, 6.0 Hz, 1H), 2.72 (s, 1H), 2.60 (dt, J=13.5, 6.8 Hz, 1H), 1.06 (s, 9H).

Step C: (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl 1H-imidazole-1-carboxylate. To a stirred mixture of (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-ol (400 mg, 0.498 mmol) and CDI (161 mg, 0.995 mmol) in THF (1 mL) at RT was added K₂CO₃ (206 mg, 1.49 mmol). The reaction mixture was stirred at 30° C. for 3 h. LCMS indicated complete reaction. The reaction mixture was concentrated under vacuum. The residue was subjected to flash chromatography (silica gel, 1:1 EtOAc/petroleum ether) to give the desired product (200 mg, 40%) as a white solid. LCMS (ESI) m/z calcd for: C₅₂H₄₈FN₇O₅Si: 897. Found: 898 (M+1)⁺.

Step D: 2-(((((2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl)oxy)carbonyl)oxy)propane-1,3-diyl dinonanoate. To a mixture of (2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl 1H-imidazole-1-carboxylate (240 mg, 0.267 mmol) and 2-hydroxypropane-1,3-diyl dinonanoate (398 mg, 1.07 mmol, prepared as described herein for the synthesis of 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dinonanoate, step B) in DMF (5 mL) stirred at RT was added DIEA (0.933 mL, 5.34 mmol). The reaction mixture was stirred at 60° C. for 16 h. LCMS indicated complete reaction. The reaction was diluted with water (50 mL), and extracted with EtOAc (3×50 mL). The organic phase was washed with saturated brine 50 mL, dried over Na₂SO₄ and evaporated under vacuum to give a residue. The residue was purified by flash chromatography (silica gel, 3:1 EtOAc/petroleum ether) to give the desired product (210 mg, 59%) as a white solid. LCMS (ESI) m/z calcd for: C₇₀H₈₄FN₅O₁₀Si: 1202. Found: 1203 (M+1)⁺. 1H NMR (400 MHz, DMSO) 58.37 (s, 1H), 8.03 (s, 1H), 7.60-7.57 (m, 2H), 7.54-7.51 (m, 2H), 7.43-7.34 (m, 3H), 7.29-7.24 (m, 14H), 7.23-7.15 (m, 5H), 6.86-6.82 (m, 2H), 6.41-6.39 (m, 1H), 5.81-5.76 (m, 1H), 4.01-3.98 (m, 4H), 3.81-3.75 (m, 2H), 3.72-3.71 (m, 4H), 2.33-2.28 (m, 6H), 1.55-1.47 (m, 6H) 1.32-1.29 (m, 24H), 0.89-0.87 (m, 6H).

Step E: 2-(((((2R,3S,5R)-2-ethynyl-5-(2-fluoro-6-(((4-methoxyphenyl)diphenylmethyl)amino)-9H-purin-9-yl)-2-(hydroxymethyl)tetrahydrofuran-3-yl)oxy)carbonyl)oxy)propane-1,3-diyl dinonanoate. To 2-(((((2R,3S,5R)-2-(((tert-butyldiphenylsilyl)oxy)methyl)-2-ethynyl-5-(2-fluoro-6-(((4-methoxyphenyl)diphenylmethyl)amino)-9H-purin-9-yl)tetrahydrofuran-3-yl)oxy)carbonyl)oxy)propane-1,3-diyl dinonanoate (500 mg, 0.416 mmol) dissolved in THF (25 mL) stirred at room temperature was added TBAF (0.42 mL, 0.42 mmol, 1N in THF). The reaction mixture was stirred at RT for 1 h. LCMS indicated complete reaction. The reaction was diluted with water (100 mL), and extracted with EtOAc (3×100 mL). The organic phase was washed with saturated aqueous NH₄Cl (2×100 mL), dried over Na₂SO₄ and evaporated in vacuo to give a residue. The residue was purified by flash chromatography (silica gel, 1:1 EtOAc/petroleum ether) to give the desired (50 mg, 13% yield) as colorless oil. LCMS (ESI) m/z calcd for: C₅₄H₆₆FN₅O₁₀: 963. Found: 964 (M+1)⁺.

Step F: 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl dinonanoate. To a mixture of 2-(((((2R,3S,5R)-2-ethynyl-5-(2-fluoro-6-(((4-methoxyphenyl)diphenylmethyl)amino)-9H-purin-9-yl)-2-(hydroxymethyl)tetrahydrofuran-3-yl)oxy)carbonyl)oxy)propane-1,3-diyl dinonanoate (50 mg, 0.052 mmol) in DCM (2 mL) was added TFA (0.2 ml). The reaction mixture was stirred at RT for 1 h. LCMS indicated complete reaction. The reaction was diluted with MeOH (5 mL) and concentrated to dryness at reduced pressure. The crude residue was subjected to RP-HPLC purification (C18, MeCN/water, 0.1% formic acid) to afford the title compound (11 mg, 31%). LCMS (ESI) m/z calcd for: C₃₄H₅₀FN₅O₉: 691. Found: 692 (M+1)⁺. ¹H NMR (400 MHz, METHANOL-d₄) δ 8.35 (s, 1H), 6.46-6.42 (m, 1H), 5.59-5.57 (m, 1H), 5.16-5.13 (m, 1H), 4.45-4.37 (m, 2H), 4.26-4.21 (m, 2H), 3.89-3.83 (m, 2H), 3.15 (s, 1H), 3.10-3.03 (m, 1H), 2.74-2.69 (m, 1H), 2.38-2.34 (m, 4H), 1.63-1.60 (m, 4H), 1.32-1.29 (m, 20H), 0.89-0.87 (m, 6H).

### Example 31: (2R,3S,5R)-5-(6-amino-2-fluoro-9H-purin-9-yl)-2-ethynyl-2-(hydroxymethyl)tetrahydrofuran-3-yl (6,18-dioxo-1,5-dioxacyclooctadecan-3-yl) carbonate

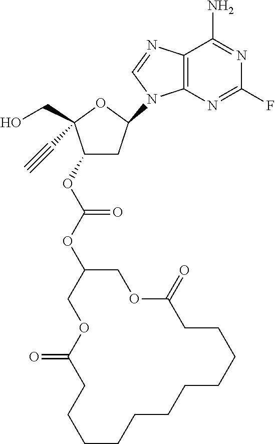 [SMILES: C#C[C@]1(CO)O[C@@H](n2cnc3c(N)nc(F)nc32)C[C@@H]1OC(=O)OC1COC(=O)CCCCCCCCCCCC(=O)OC1]

The title compound was prepared as described herein for the synthesis of 2-(((((2R,3S,5R)-5-(6-amino-2-fluoro-9H-purin-9-yl)-2-ethynyl-2-(hydroxymethyl)tetrahydrofuran-3-yl)oxy)carbonyl)oxy)propane-1,3-diyl dinonanoate, substituting 3-hydroxy-1,5-dioxacyclooctadecane-6,18-dione for 2-hydroxypropane-1,3-diyl dinonanoate in step D. LCMS (ESI) m/z calcd for C₂₉H₃₈FN₅O₉: 619. Found: 620 (M+1)⁺. ¹H NMR (400 MHz, CDCl₃) δ 7.82 (s, 1H), 6.35 (dd, J=9.6, 5.3 Hz, 1H), 6.06 (br, s, 2H), 5.65 (d, J=5.6 Hz, 1H), 5.13-5.08 (m, 1H), 4.49-4.38 (m, 2H), 4.27-4.23 (m, 2H), 4.06 (d, J=12.8 Hz, 1H), 3.95 (d, J=12.8 Hz, 1H), 3.29-3.21 (m, 1H), 2.70 (s, 1H), 2.57 (dd, J=13.6, 5.2 Hz, 1H), 2.40-2.35 (m, 4H), 1.68-1.64 (m, 4H), 1.40-1.24 (m, 14H).

### Example 32: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dipentanoate

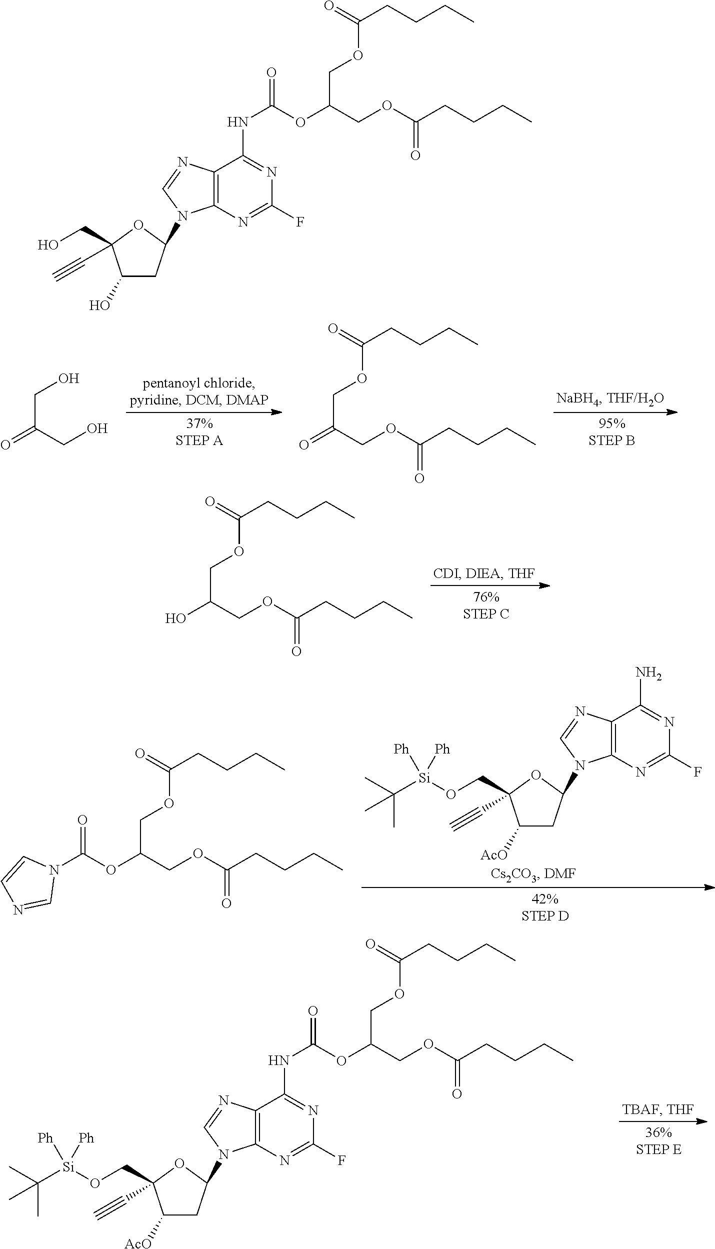

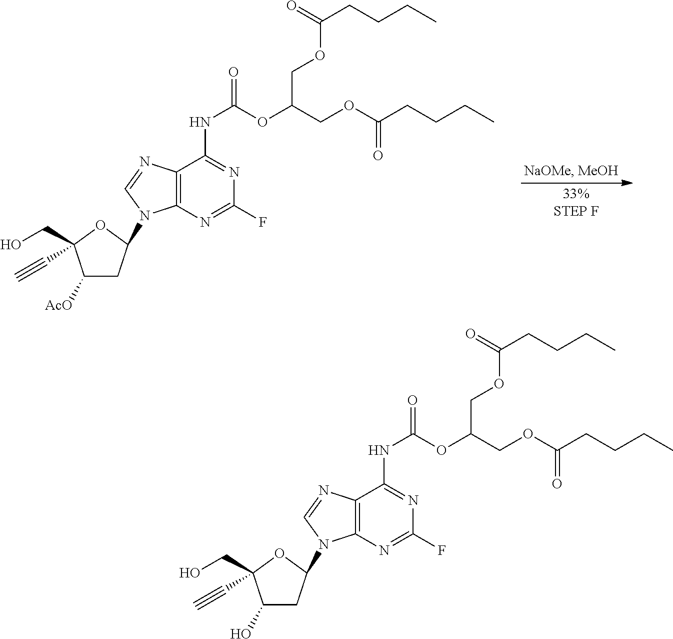 [SMILES: C#C[C@]1(CO)O[C@@H](n2cnc3c(NC(=O)OC(COC(=O)CCCC)COC(=O)CCCC)nc(F)nc32)C[C@@H]1OC(C)=O ; C#C[C@]1(CO)O[C@@H](n2cnc3c(NC(=O)OC(COC(=O)CCCC)COC(=O)CCCC)nc(F)nc32)C[C@@H]1O]

Step A: 2-oxopropane-1,3-diyl dipentanoate. 1,3-Dihydroxypropan-2-one (10.0 g, 111 mmol) was dissolved in DCM (150 mL). To the solution was added pyridine (26.3 g, 333 mmol) and pentanoyl chloride (29.4 g, 244 mmol) was added at 0° C. The reaction mixture was stirred at RT overnight. TLC indicated complete reaction. The reaction mixture was filtered and the solid was washed with dichloromethane. The filtrate was evaporated in vacuum to remove DCM. The residue was extracted with EtOAc (250 mL) and water (200 mL). The organic phase was washed sequentially with saturated brine (200 mL), saturated aqueous NaHCO₃ (200 mL), 0.1 M aqueous HCl (200 mL), saturated brine (200 mL), dried over sodium sulfate and evaporated in vacuum to give the crude product as yellow oil. The residue was subjected to flash chromatography (silica gel, 3:1 petroleum ether/EtOAc) to give the desired product (10.5 g, 37%) as a white solid. LCMS (ESI) m/z calcd for C₁₃H₂₂O₅: 258. Found: 259 (M+1)⁺. ¹H NMR (400 MHz, CDCl₃) δ 4.74 (s, 4H), 2.42 (t, J=7.6 Hz, 4H), 1.67-1.58 (m, 4H), 1.43-1.28 (m, 4H), 0.91 (t, J=7.2 Hz, 6H).

Step B: 2-hydroxypropane-1,3-diyl dipentanoate. 2-Oxopropane-1,3-diyl dipentanoate (5.50 g, 21.3 mmol) was dissolved in THF (45 mL)/water (3 mL). The resulting solution was treated with NaBH₄ (1.6 g, 42.6 mmol) in several portions at 0° C. The resulting mixture was stirred for 40 minutes at RT. TLC indicated complete reaction. The reaction mixture was quenched by adding approximately 6 mL of glacial acetic acid. The solvent was evaporated in vacuum to remove THF. The residue was partitioned between EtOAc (100 mL) and water (100 mL). The organic phase was washed with saturated brine (100 mL), dried over Na₂SO₄ and evaporated in vacuum to give the desired product (25 g, 95%) as a white solid. LCMS (ESI) m/z calcd for C₁₃H₂₄O₅: 260. Found: 261 (M+1)⁺. ¹H NMR (400 MHz, DMSO-d₆) δ 5.25 (d, J=5.2 Hz, 1H), 4.10-3.90 (m, 4H), 2.32-2.22 (m, 4H), 1.52-1.43 (m, 4H), 1.34-1.19 (m, 4H), 0.85 (t, J=7.2 Hz, 6H).

Step C: 2-((1H-imidazole-1-carbonyl)oxy)propane-1,3-diyl dipentanoate. 2-Hydroxypropane-1,3-diyl dipentanoate (3.0 g, 11.5 mmol) was dissolved in THF (30 mL). CDI (5.61 g, 34.6 mmol) and DIEA (6.04 ml, 34.6 mmol) were added. The resulting mixture was stirred for 16 h at RT. LCMS indicated complete reaction. The reaction mixture was concentrated under vacuum. The residue was subjected to preparative TLC (silica gel, 1:1 EtOAc/petroleum ether) to give the desired product (1.2 g, 76%) as an off white oil. LCMS (ESI) m/z calcd for C₁₇H₂₆N₂O₆: 354. Found: 355 (M+1)⁺. ¹H NMR (300 MHz, DMSO-d₆) δ 8.23 (t, J=1.2 Hz, 1H), 7.56 (t, J=1.5 Hz, 1H), 7.09 (dd, J=1.5, 0.9 Hz, 1H), 5.42-5.36 (m, 1H), 4.45 (dd, J=12.3, 3.6 Hz, 2H), 4.33 (dd, J=12.3, 6.6 Hz, 2H), 2.31 (t, J=7.2 Hz, 4H), 1.54-1.38 (m, 4H), 1.32-1.12 (m, 4H), 0.80 (t, J=7.2 Hz, 6H).

Step D: 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)propane-1,3-diyl dipentanoate. (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (1.00 g, 1.74 mmol), and Cs₂CO₃ (1704 mg, 5.23 mmol) was dissolved in DMF (10 mL). To the solution was added 2-((1H-imidazole-1-carbonyl)oxy)propane-1,3-diyl dipentanoate (803 mg, 2.27 mmol) at 0° C. The resulting mixture was stirred for 2 h at RT. LCMS indicated complete reaction. The reaction was diluted with water, and extracted with EtOAc (3×30 mL). The organic phases were combined, washed with saturated brine, dried over Na₂SO₄ and concentrated under vacuum. The residue was subjected to flash chromatography (silica gel, 1:1 EtOAc/petroleum ether) to give the desired product (700 mg, 42%) as a white solid. LCMS (ESI) m/z calcd for C₄₄H₅₄FN₅O₁₀Si: 859. Found: 860 (M+1)⁺. ¹H NMR (400 MHz, DMSO-d₆) δ 11.12 (s, 1H), 8.56 (s, 1H), 7.55 (t, J=6.4 Hz, 4H), 7.44-7.35 (m, 2H), 7.31 (t, J=7.2 Hz, 4H), 6.45 (t, J=6.8 Hz, 1H), 5.83 (dd, J=7.2, 5.2 Hz, 1H), 5.23-5.21 (m, 1H), 4.33-4.28 (m, 2H), 4.23-4.17 (m, 2H), 3.92 (d, J=10.4 Hz, 1H), 3.77 (d, J=17.2 Hz, 1H), 3.74 (s, 1H), 3.25-3.18 (m, 1H), 2.68-2.60 (m, 1H), 2.29 (t, J=7.2 Hz, 4H), 2.11 (s, 3H), 1.50-1.42 (m, 4H), 1.29-1.12 (m, 4H), 0.92 (s, 9H), 0.77 (t, J=7.2 Hz, 6H).

Step E: 2-(((9-((2R,4S,5R)-4-acetoxy-5-ethynyl-5-(hydroxymethyl)tetrahydrofuran-2-yl)-2-fluoro-9H-purin-6-yl)carbamoyl)oxy) propane-1,3-diyl dipentanoate. 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)propane-1,3-diyl dipentanoate (1400 mg, 1.63 mmol) was dissolved in THF (30 mL). The resulting solution was treated with TBAF (3.26 mL, 1 M in THF, 3.26 mmol) and stirred for 1 h at RT. LCMS indicated complete reaction. The reaction mixture was concentrated under vacuum. Water was added, the mixture was extracted with EtOAc (3×30 mL). The organic phases were combined, washed with brine, dried over Na₂SO₄ and concentrated under vacuum. The residue was subjected to preparative TLC (silica gel, 10:1 DCM/MeOH) to give impure product. This material was subjected to preparative SFC chromatography (CHIRALPAK IG, 20×250 mm column, 5 um; Mobile Phase A: CO₂, Mobile Phase B: 2 mM NH₃/MeOH; Flow rate: 40 mL/min; isocratic at 50% B) to give the desired product (450 mg, 36%). LCMS (ESI) m/z calcd for C₂₈H₃₆FN₅O₁₀: 621. Found: 622 (M+1)*. ¹H NMR (400 MHz, CDCl₃) δ 8.48 (s, 1H), 8.15 (s, 1H), 6.44-6.40 (m, 1H), 5.80-5.76 (m, 1H), 5.39-5.34 (m, 1H), 4.40-4.36 (m, 2H), 4.08-3.88 (m, 2H), 4.04 (d, J=12 Hz, 1H), 3.94 (d, J=12 Hz, 1H), 3.18-3.12 (m, 1H), 2.66 (s, 1H), 2.60-2.52 (m, 1H), 2.34 (t, J=7.6 Hz, 4H), 2.18 (s, 3H), 1.64-1.55 (m, 4H), 1.40-1.21 (m, 4H), 0.89 (t, J=7.3 Hz, 6H).

Step F: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dipentanoate. To a solution of 2-(((9-((2R,4S,5R)-4-acetoxy-5-ethynyl-5-(hydroxymethyl)tetrahydrofuran-2-yl)-2-fluoro-9H-purin-6-yl)carbamoyl)oxy)propane-1,3-diyl dipentanoate (400 mg, 0.643 mmol) in 1:1 THF/MeOH (20 mL) was added sodium methoxide (70 mg, 0.39 mmol). After stirring at RT for 1 h, the reaction mixture was quenched with AcOH (30 mL) and concentrated under vacuum. The residue was subjected to RP-HPLC purification (C18, MeCN/water, 0.05% formic acid) to afford the title compound (120 mg, 32%), LCMS (ESI) m/z calcd for C₂₆H₃₄FN₅O₉: 579. Found: 580 (M+1)⁺. ¹H NMR (400 MHz, METHANOL-d₄) δ 8.50 (s, 1H), 6.42 (dd, J=7.6, 4.4 Hz, 1H), 5.39-5.34 (m, 1H), 4.78 (t, J=7.2 Hz, 1H), 4.41 (dd, J=12.0, 3.6 Hz, 2H), 4.30 (dd, J=12.0, 6.4 Hz, 2H), 3.86-3.75 (m, 2H), 3.11 (s, 1H), 2.84-2.78 (m, 1H), 2.70-2.63 (m, 1H), 2.36 (t, J=7.2 Hz, 4H), 1.62-1.55 (m, 4H), 1.38-1.28 (m, 4H), 0.88 (t, J=7.6 Hz, 6H).

### Example 33: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dinonanoate

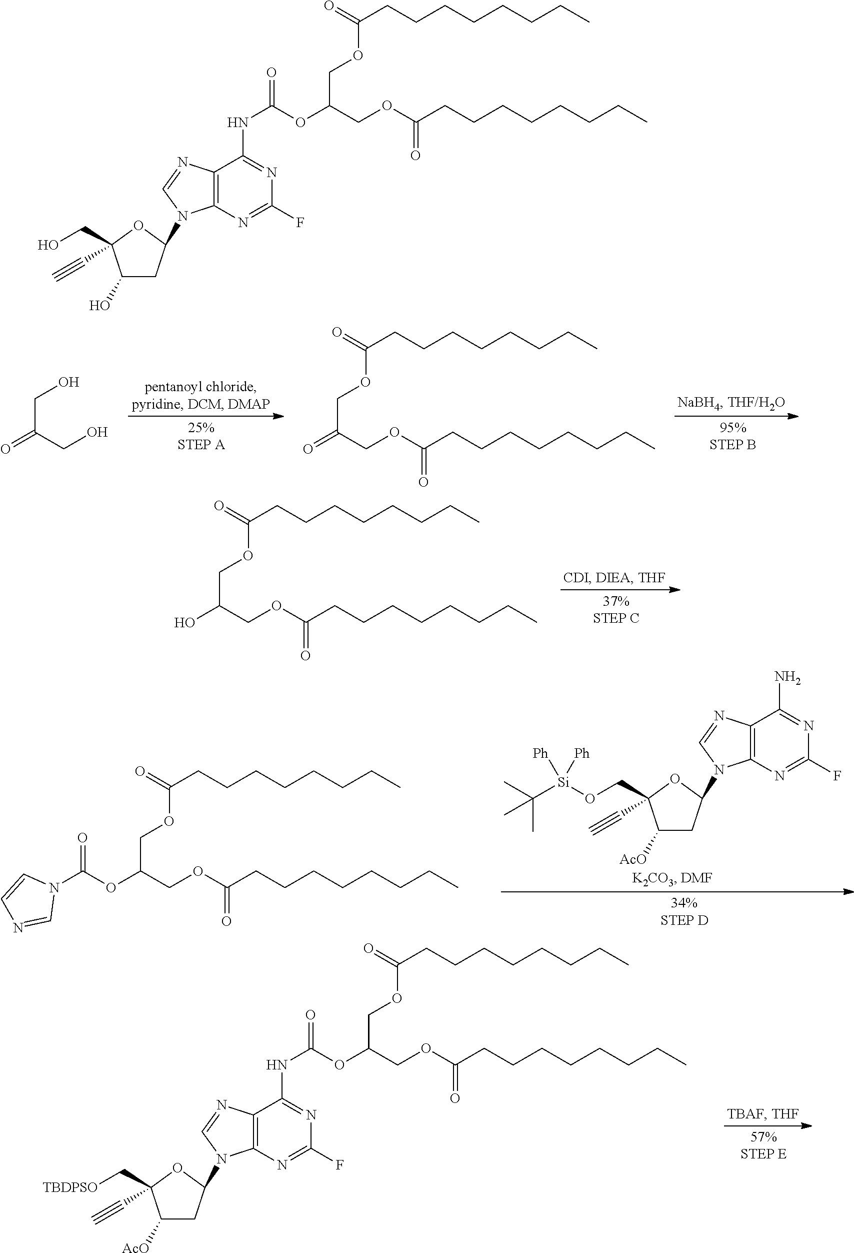

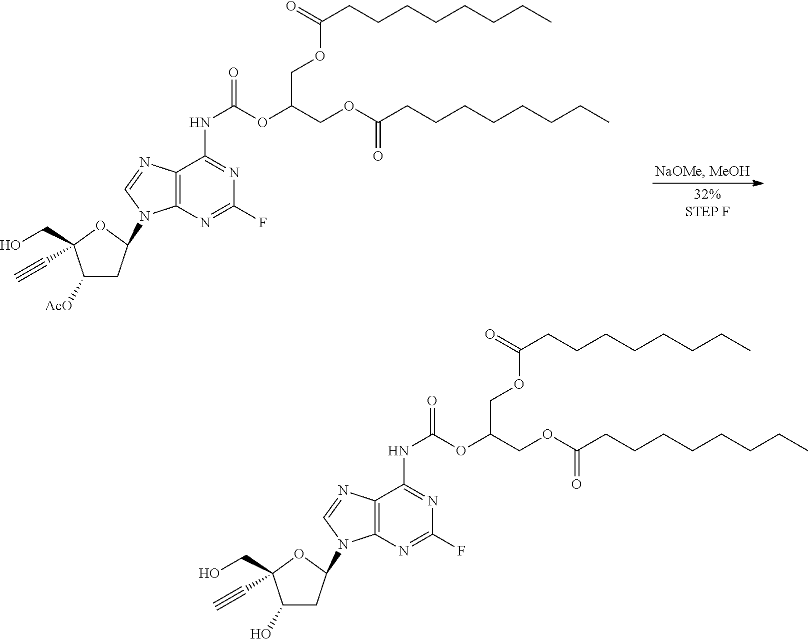 [SMILES: C#C[C@]1(CO)O[C@@H](n2cnc3c(NC(=O)OC(COC(=O)CCCCCCCC)COC(=O)CCCCCCCC)nc(F)nc32)C[C@@H]1OC(C)=O ; C#C[C@]1(CO)O[C@@H](n2cnc3c(NC(=O)OC(COC(=O)CCCCCCCC)COC(=O)CCCCCCCC)nc(F)nc32)C[C@@H]1O]

Step A: 2-oxopropane-1,3-diyl dinonanoate. To a solution of 1,3-dihydroxypropan-2-one (20.0 g, 222 mmol) in DCM (300 mL) at 0° C. was added pyridine (52.6 g, 666 mmol) followed by nonanoyl chloride (85.9 g, 488 mmol). The reaction mixture was stirred at RT overnight. The reaction mixture was filtered and the solid was washed with DCM. The filtrate was evaporated in vacuum to remove DCM. The residue was partitioned between EtOAc (500 mL) and water (500 mL). The organic phase was washed sequentially with saturated brine (400 mL), saturated aqueous Na₂CO₃ (400 mL), 0.1 M aqueous HCl (400 mL) and saturated brine 400 mL. After drying over Na₂SO₄, the solution was concentrated at reduced pressure to afford a yellow oil. The residue was subjected to flash chromatography (silica gel, 3:1 EtOAc/petroleum ether) to give the desired product (23 g, 25%) as a white solid. LCMS (ESI) m/z calcd for C₂₁H₃₈O₅: 370. Found: 371 (M+1)⁺. ¹H NMR (300 MHz, CDCl₃) δ 4.75 (s, 4H), 2.48-2.27 (m, 4H), 1.72-1.60 (m, 4H), 1.37-1.26 (m, 20H), 0.93-0.83 (m, 6H).

Step B: 2-hydroxypropane-1,3-diyl dinonanoate. To a solution of 2-oxopropane-1,3-diyl dinonanoate (22.0 g, 59.4 mmol) in THF (30 mL)/water (3 mL) at 0° C. was added NaBH₄ (3.37 g, 89 mmol) in several portions. The resulting mixture was stirred for 40 minutes at RT. TLC indicated complete reaction. The reaction mixture was mixed with water and extracted with EtOAc (3×50 mL). The combined EtOAc extracts were washed with brine, dried over Na₂SO₄, and concentrated under vacuum to give the desired product (21 g, 95%) as a white solid. LCMS (ESI) m/z calcd for C₂₁H₄₀O₅: 372. Found: 373 (M+1)⁺. ¹H NMR (400 MHz, DMSO-d₆) δ 5.26 (d, J=5.2 Hz, 1H), 5.02-4.81 (m, 1H), 4.17-3.80 (m, 4H), 2.35-2.23 (m, 4H), 1.56-1.49 (m, 4H), 1.30-1.67 (m, 20H), 0.86 (t, J=6.6 Hz, 6H).

Step C: 2-((1H-imidazole-1-carbonyl)oxy)propane-1,3-diyl dinonanoate. 2-hydroxypropane-1,3-diyl dinonanoate (1000 mg, 2.68 mmol) was dissolved in THF (10 mL) and the solution treated with CDI (1306 mg, 8.05 mmol) followed by DIEA (1.41 mL, 8.05 mmol). The resulting mixture was stirred for 16 h at RT. LCMS indicated complete reaction. The reaction mixture was concentrated under vacuum. The residue was subjected to preparative TLC (silica gel, 1:1 EtOAc/petroleum ether) to give the desired product (500 mg, 37%) as off white oil. LCMS (ESI) m/z calcd for C₂₅H₄₂N₂O₆: 466. Found: 467 (M+1)⁺. ¹H NMR (400 MHz, DMSO-d₆) δ 8.20 (s, 1H), 7.54 (t, J=1.6 Hz, 1H), 7.10-7.05 (m, 1H), 5.39-5.34 (m, 1H), 4.43 (dd, J=12.4, 3.6 Hz, 2H), 4.31 (dd, J=12.4, 6.4 Hz, 2H), 2.30-2.26 (m, 4H), 1.49-1.42 (m, 4H), 1.27-1.15 (m, 20H), 0.82 (t, J=7.6 Hz, 6H).

Step D: 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)propane-1,3-diyl dinonanoate. To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (800 mg, 1.394 mmol), in DMF (10 mL) at 0° C. was added K₂CO₃ (193 mg, 1.39 mmol) followed by 2-((1H-imidazole-1-carbonyl)oxy)propane-1,3-diyl dinonanoate (976 mg, 2.09 mmol). The resulting mixture was stirred for 60 h at RT. LCMS indicated complete reaction. The reaction was diluted with water, and extracted with EA (3×20 mL). The organic phases were combined, washed with saturated brine, dried over Na₂SO₄ and concentrated under vacuum. The residue was subjected to flash chromatography (silica gel, 1:1 EtOAc/petroleum ether) to give the desired product (500 mg, 34%) as a white solid. LCMS (ESI) m/z calcd for C₅₂H₇₀FN₅O₁₀Si: 971. Found: 972 (M+1)⁺. ¹H NMR (400 MHz, DMSO-d₆) δ 11.13 (s, 1H), 8.56 (s, 1H), 7.57-7.53 (m, 4H), 7.45-7.35 (m, 2H), 7.33-7.28 (m, 4H), 6.45 (t, J=6.8 Hz, 1H), 5.84-5.81 (m, 1H), 5.26-5.21 (m, 1H), 4.34-4.15 (m, 4H), 3.93 (d, J=10.8 Hz, 1H), 3.76 (d, J=10.8 Hz, 1H), 3.74 (s, 1H), 3.24-3.16 (m, 1H), 2.66-2.60 (m, 1H), 2.28 (t, J=7.2 Hz, 4H), 2.11 (s, 3H), 1.50-1.43 (m, 4H), 1.26-1.08 (m, 20H), 0.93 (s, 9H), 0.87-0.72 (m, 6H).

Step E: 2-(((9-((2R,4S,5R)-4-acetoxy-5-ethynyl-5-(hydroxymethyl)tetrahydrofuran-2-yl)-2-fluoro-9H-purin-6-yl)carbamoyl)oxy)propane-1,3-diyl dinonanoate. To a solution of 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)propane-1,3-diyldinonanoate (450 mg, 0.463 mmol) in THF (20 mL) was added TBAF (0.93 mL, 1M in THF, 0.93 mmol). The resulting mixture was stirred for 1 h at RT. LCMS indicated complete reaction. The reaction mixture was concentrated under vacuum. Water was added and the mixture was extracted with EA (3×20 mL). The organic phases were combined, washed with brine, dried over Na₂SO₄ and concentrated under vacuum. The residue was subjected to preparative TLC (10:1 DCM/MeOH) to give the impure product. This material was subjected to preparative SFC (CHIRALPAK IF-2, 2×25 cm column, 5 um; Mobile Phase A: CO₂, Mobile Phase B: EtOH; Flow rate: 40 mL/min; isocratic at 40% B) to give the desired product (250 mg, 57%), LCMS (ESI) m/z calcd for C₃₅H₅₂FN₅O₁₀: 733. Found: 734 (M+1)⁺. ¹H NMR (400 MHz, CDCl₃) δ 8.33 (s, 1H), 8.10 (s, 1H), 6.42 (t, J=6.8 Hz, 1H), 5.78 (br s, 1H), 5.36 (br s, 1H), 4.38 (dd, J=12.4, 3.6 Hz, 2H), 4.27 (dd, J=12.4, 5.6 Hz, 2H), 4.04 (d, J=12.4 Hz, 1H), 3.94 (d, J=12.4 Hz, 1H), 3.14 (br s, 1H), 2.66 (s, 1H), 2.59-2.52 (m, 1H), 2.33 (t, J=7.6 Hz, 4H), 2.18 (s, 3H), 1.62 (q, J=7.2 Hz, 4H), 1.26 (d, J=10.4 Hz, 20H), 0.86 (t, J=6.4 Hz, 6H).

Step F: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)propane-1,3-diyl dinonanoate. To a solution of 2-(((9-((2R,4S,5R)-4-acetoxy-5-ethynyl-5-(hydroxymethyl)tetrahydrofuran-2-yl)-2-fluoro-9H-purin-6-yl)carbamoyl)oxy)propane-1,3-diyl dipentanoate (400 mg, 0.643 mmol) in 1:1 THF/MeOH (20 mL) was added sodium methoxide (70 mg, 0.39 mmol, 30% in MeOH). The resulting mixture was stirred for 1 h at RT. LCMS indicated complete reaction. The reaction mixture was quenched with AcOH (30 mL) and concentrated under vacuum. The residue was subjected to RP-HPLC purification (C18, MeCN/water, 0.1% formic acid) to give the desired product (120 mg, 32%), LCMS (ESI) m/z calcd for C₃₄H₅₀FN₅O₉: 691. Found: 692 (M+1)⁺. ¹H NMR (400 MHz, METHANOL-d₄) δ 8.51 (s, 1H), 6.43 (dd, J=7.2 Hz, 1H), 5.40-5.35 (m, 1H), 4.77 (t, J=7.2 Hz, 1H), 4.43-4.39 (m, 2H), 4.34-4.29 (m, 2H), 3.87-3.75 (m, 2H), 3.10 (s, 1H), 2.83-2.77 (m, 1H), 2.69-2.62 (m, 1H), 2.37-2.33 (t, J=7.2 Hz, 4H), 1.63-1.56 (m, 4H), 1.37-1.15 (m, 20H), 0.86 (t, J=6.8 Hz, 6H).

### Example 34: 6,18-dioxo-1,5-dioxacyclooctadecan-3-yl (9-((2R,4S,5R)-5-ethynyl-4-hydroxy-5-(hydroxymethyl)tetrahydrofuran-2-yl)-2-fluoro-9H-purin-6-yl)carbamate

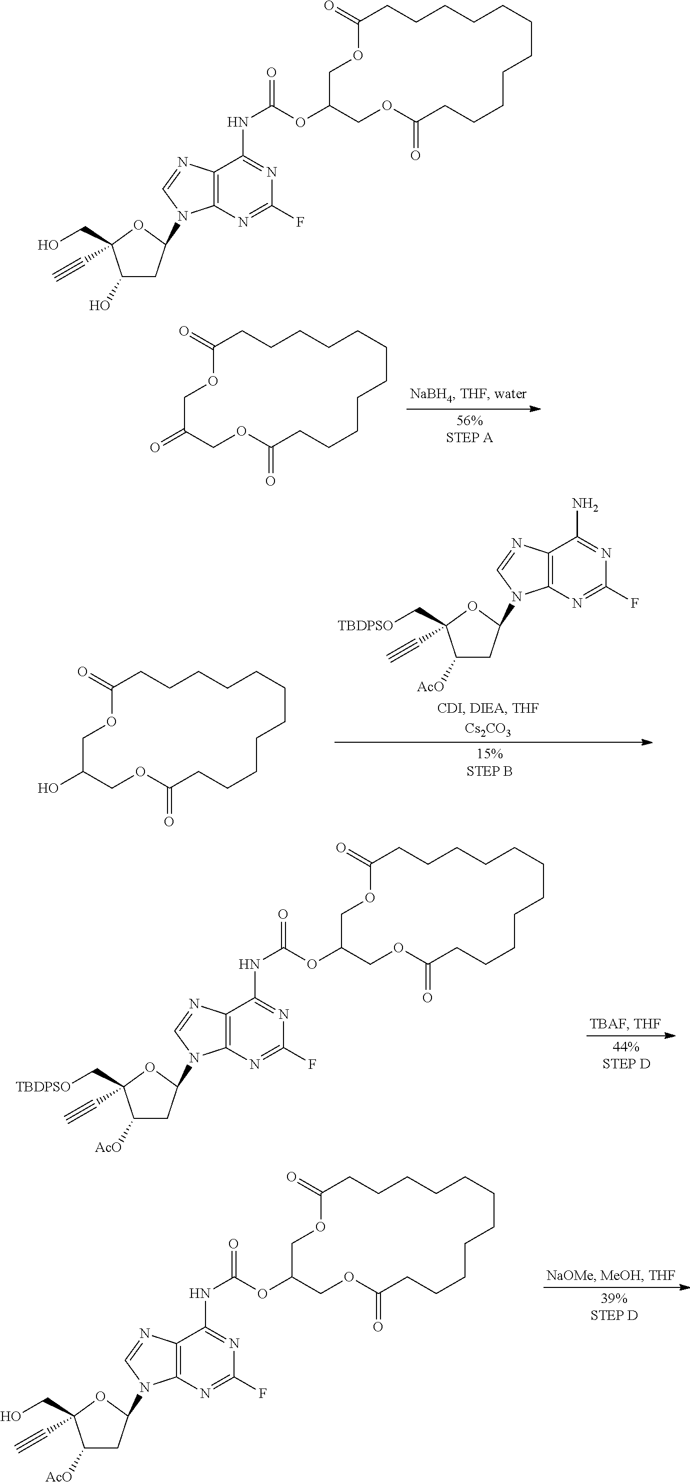

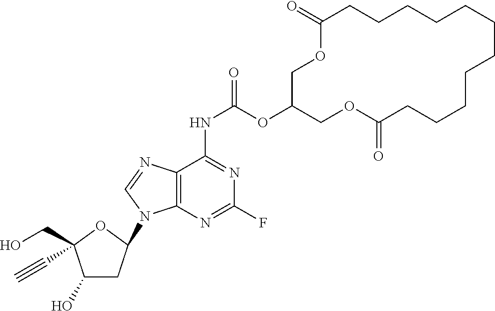 [SMILES: C#C[C@]1(CO)O[C@@H](n2cnc3c(NC(=O)OC4COC(=O)CCCCCCCCCCCC(=O)OC4)nc(F)nc32)C[C@@H]1O]

Step A: 3-hydroxy-1,5-dioxacyclooctadecane-6,18-dione. To a stirred solution of 1,5-dioxacyclooctadecane-3,6,18-trione (4.0 g, 13.41 mmol) in THF (30 mL) and water (3 mL) at 0° C. was added NaBH₄ (0.507 g, 13.4 mmol) portionwise. The reaction mixture was stirred at 15° C. for 1 h. LCMS indicated complete reaction. The reaction mixture was quenched with 1 mM aqueous HCl (50 mL) and extracted with DCM (3×50 mL). The combined organic layer was dried over Na₂SO₄ and concentrated under vacuum to give the desired product (2.5 g, 56%) as a colorless oil. LCMS (ESI) m/z calcd for C₁₆H₂₈O₅: 300. Found: 301 (M+1)⁺. ¹H NMR (400 MHz, DMSO-d₆) δ 5.34 (d, J=5.6 Hz, 1H), 5.01-4.91 (m, 1H), 5.12-3.91 (m, 4H), 1.58-1.46 (m, 4H), 1.27-1.22 (m, 18H).

Step B: (2R,3S,5R)-2-(((tert-butyldiphenylsilyl)oxy)methyl)-5-(6-((((6,18-dioxo-1,5-dioxacyclooctadecan-3-yl)oxy)carbonyl)amino)-2-fluoro-9H-purin-9-yl)-2-ethynyltetrahydrofuran-3-yl acetate. To a solution of 3-hydroxy-1,5-dioxacyclooctadecane-6,18-dione (2.00 g, 6.66 mmol)), in THF (15 mL) was added CDI (2.16 g, 13.3 mmol) and DIEA (2.33 mL, 13.3 mmol). The reaction mixture was stirred at 15° C. for 1 h. Then (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (1.91 g, 3.33 mmol) and Cs₂CO₃ (2.60 g, 7.99 mmol) were added and the reaction was stirred at 15° C. for 2 h. LCMS indicated complete reaction. The solvent was concentrated under vacuum. The residue was subjected to flash chromatography (silica gel, 2:1 EtOAc/petroleum ether) to give the desired product (1.0 g, 15%) as a colorless oil. LCMS (ESI) m/z calcd for C₄₇H₅₈FN₅O₁₀Si: 899. Found: 900 (M+1)⁺. ¹H NMR (400 MHz, CDCl₃) δ 8.16 (s, 1H), 8.06 (s, 1H), 7.66-7.63 (m, 4H), 7.50-7.32 (m, 6H), 6.53 (t, J=6.8 Hz, 1H), 5.81 (dd, J=6.8, 4.0 Hz, 1H), 5.34-5.29 (m, 1H), 4.48-4.38 (m, 2H), 4.29 (dd, J=11.6, 4.8 Hz, 2H), 4.12 (dd, J=14.4, 7.2 Hz, 1H), 4.05 (d, J=11.2 Hz, 1H), 3.95 (d, J=11.2 Hz, 1H), 2.88-2.68 (m, 2H), 2.39-2.32 (m, 4H), 2.16 (s, 3H), 1.67-1.62 (m, 4H), 1.34-1.24 (m, 14H), 1.08 (s, 9H).

Step C: (2R,3S,5R)-5-(6-((((6,18-dioxo-1,5-dioxacyclooctadecan-3-yl)oxy)carbonyl)amino)-2-fluoro-9H-purin-9-yl)-2-ethynyl-2-(hydroxymethyl)tetrahydrofuran-3-yl acetate. To a solution of (2R,3S,5R)-2-(((tert-butyldiphenylsilyl)oxy)methyl)-5-(6-((((6,18-dioxo-1,5-dioxacyclooctadecan-3-yl)oxy)carbonyl)amino)-2-fluoro-9H-purin-9-yl)-2-ethynyltetrahydrofuran-3-yl acetate (1.00 g, 1.11 mmol) in THF (5 mL) was added TBAF (2.22 mL, 2.22 mmol, 1N in THF). The reaction mixture was stirred at 20° C. for 16 h. LCMS indicated complete reaction. The reaction was diluted with water (50 mL) and the mixture was extracted with EtOAc (3×60 mL). The combined organic layer was dried over Na₂SO₄ and concentrated under vacuum. The residue was subjected to flash chromatography (silica gel, 10:1 DCM/MeOH) followed by preparative HPLC (CHIRALPAK IE, 2×25 cm column, 5 um; Mobile Phase A: MTBE containing 10 mM NH₃/MeOH), Mobile Phase B: EtOH; Flow rate: 20 mL/min; isocratic at 15% B over 11 min) to give the title compound (370 mg, 44%) as a colorless oil. LCMS (ESI) m/z calcd for C₃₁H₄₀FN₅O₁₀: 661. Found: 662 (M+1)⁺. ¹H NMR (400 MHz, CDCl₃) δ 8.18 (s, 1H), 8.05 (s, 1H), 6.41 (dd, J=8.8, 5.6 Hz, 1H), 5.78 (dd, J=6.4, 2.4 Hz, 1H), 5.35-5.30 (m, 1H), 4.74 (dd, J=10.8, 3.6 Hz, 1H), 4.46-4.40 (m, 2H), 4.32-4.27 (m, 2H), 4.15-3.91 (m, 2H), 3.19-3.12 (m, 1H), 2.65 (s, 1H), 2.57-2.52 (m, 1H), 2.46-2.27 (m, 4H), 2.18 (s, 3H), 1.67-1.62 (m, 4H), 1.34-1.24 (m, 14H).

Step D: 6,18-dioxo-1,5-dioxacyclooctadecan-3-yl (9-((2R,4S,5R)-5-ethynyl-4-hydroxy-5-(hydroxymethyl)tetrahydrofuran-2-yl)-2-fluoro-9H-purin-6-yl)carbamate. To a solution of (2R,3S,5R)-5-(6-((((6,18-dioxo-1,5-dioxacyclooctadecan-3-yl)oxy)carbonyl)amino)-2-fluoro-9H-purin-9-yl)-2-ethynyl-2-(hydroxymethyl)tetrahydrofuran-3-yl acetate (370 mg, 0.559 mmol) in 1:1 THF/MeOH (10 mL) was added sodium methoxide (60.4 mg, 0.336 mmol) in methanol (5 mL) dropwise over 1 min. The resulting mixture was stirred for 30 min at 20° C. LCMS indicated complete reaction. The reaction was quenched with water (30 mL) and the mixture was extracted with EtOAc (3×30 mL). The combined organic layer was dried over Na₂SO₄ and concentrated under vacuum. The residue was subjected to RP-HPLC purification (C18, MeCN/water, 0.1% formic acid)) to give the desired product (140 mg, 39%) as a white solid. LCMS (ESI) m/z calcd for C₂₉H₃₈FN₅O₉: 619. Found: 620 (M+1)⁺. ¹H NMR (400 MHz, CDCl₃) δ 8.56 (s, 1H), 8.06 (s, 1H), 6.41 (dd, J=8.0, 6.0 Hz, 1H), 5.31 (dd, J=10.4, 5.2 Hz, 1H), 4.76 (dd, J=5.2, 3.6 Hz, 1H), 4.45-4.29 (m, 4H), 4.06 (d, J=12.8 Hz, 1H), 3.88 (d, J=12.8 Hz, 1H), 3.09-3.02 (m, 1H), 2.81 (s, 1H), 2.58-2.53 (m, 1H), 2.40-2.36 (m, 4H), 1.67-1.63 (m, 4H), 1.39-1.21 (m, 14H).

### Examples 35 and 36: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)-3-(nonyloxy)propyl nonanoate—diastereomers 1 and 2

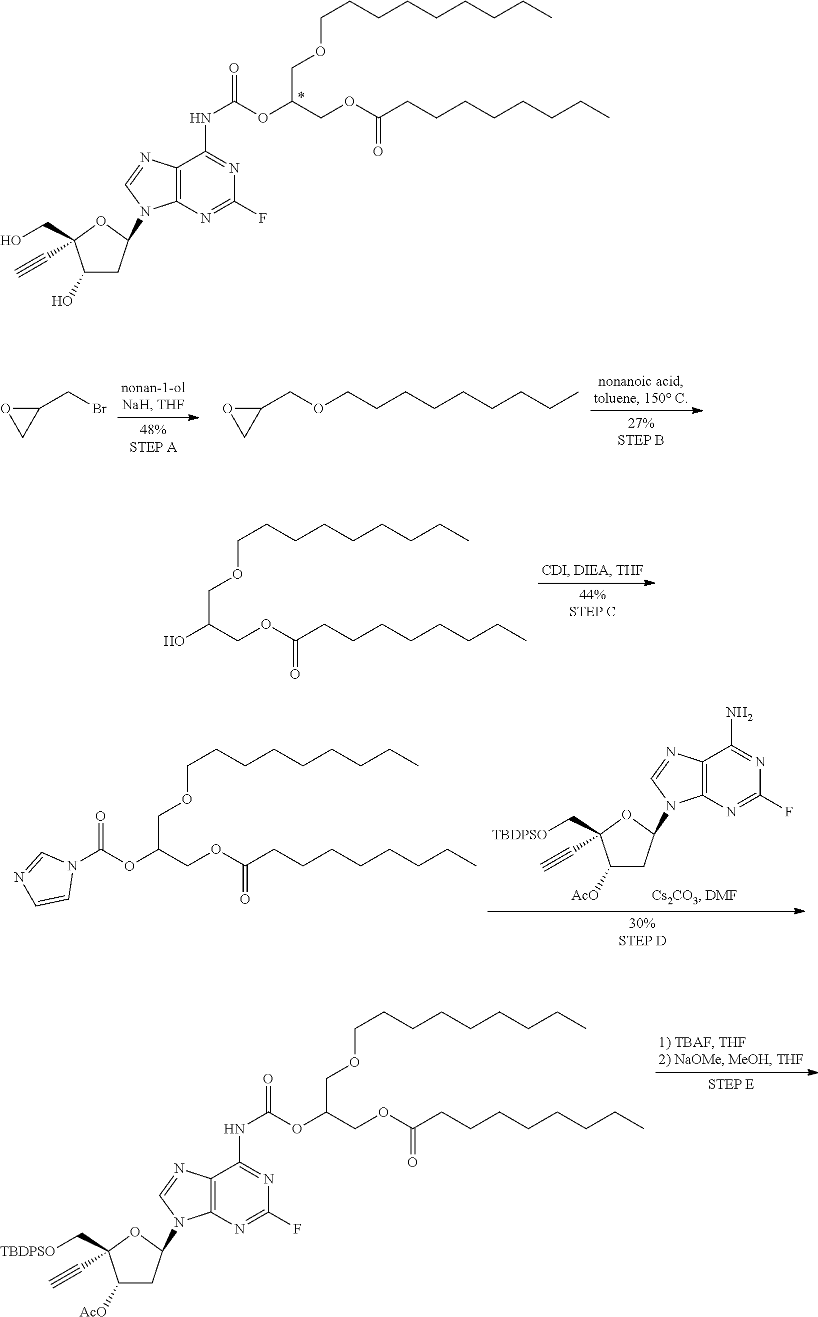

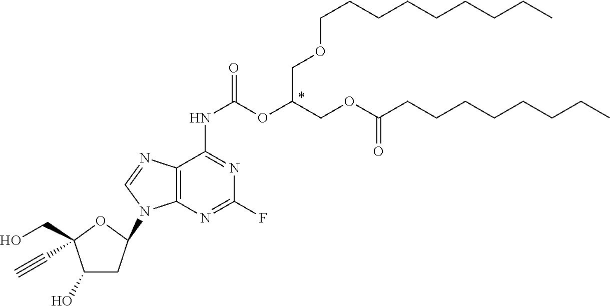 [SMILES: C#C[C@]1(CO)O[C@@H](n2cnc3c(NC(=O)OC(COCCCCCCCCC)COC(=O)CCCCCCCC)nc(F)nc32)C[C@@H]1O]

Step A: 2-((nonyloxy)methyl)oxirane. Nonan-1-ol (15.0 g, 104 mmol) was dissolved in THF (50 mL), and the solution treated with sodium hydride (10.4 g, 156 mmol, 60%). After stirring at 0° C. for 1 h, 2-(bromomethyl)oxirane (5.22 g, 38.1 mmol) was added with dropwise. After 5 h at RT, the reaction mixture was diluted with water (100 mL) and extracted with EtOAc (3×50 mL). The combined EtOAc extracts were dried over Na₂SO₄ and concentrated at reduced pressure. The crude material was subjected to flash chromatography (silica gel, 10:1 petroleum ether/EtOAc)) to give the desired product (11.0 g, 48%) as a yellow oil. ¹H NMR (400 MHz, CDCl₃) δ 3.70 (dd, J=11.2, 2.8 Hz, 1H), 3.54-3.33 (m, 3H), 3.16-3.09 (m, 1H), 2.79 (t, J=4.8 Hz, 1H), 2.64-2.53 (m, 1H), 1.64-1.51 (m, 2H), 1.34-1.26 (m, 12H), 0.92-0.79 (m, 3H).

Step B: 2-hydroxy-3-(nonyloxy)propyl nonanoate. To a solution of 2-((nonyloxy)methyl)oxirane (8 g, 39.9 mmol) in toluene (15 mL) was added nonanoic acid (18.96 g, 120 mmol), and the resulting mixture was stirred for 40 h at 150° C. LCMS indicated complete reaction. The solvent was removed in vacuo. The residue was partioned between DCM (100 mL) and aqueous K₂CO₃ and the phases separated. The organic phase was dried over Na₂SO₄ and concentrated under vacuum. The residue was subjected to flash chromatography (silica gel, 8:1 petroleum ether/EtOAc) to give the desired product (4.5 g, 27%) as a yellow oil. LCMS (ESI) m/z calcd for C₂₁H₄₂O₄: 358. Found: 359 (M+1)⁺. ¹H NMR (400 MHz, CDCl₃) δ 4.22-4.06 (m, 2H), 4.02-3.97 (m, 1H), 3.55-3.34 (m, 4H), 2.34 (t, J=7.6 Hz, 2H), 1.64-1.53 (m, 4H), 1.33-1.14 (m, 22H), 0.89-0.83 (m, 6H).

Step C: 1-(nonanoyloxy)-3-(nonyloxy)propan-2-yl 1H-imidazole-1-carboxylate. 2-hydroxy-3-(nonyloxy)propyl nonanoate (4.5 g, 12.55 mmol) was dissolved in THF (30 mL), and the solution treated with CDI (4.07 g, 25.1 mmol) and DIEA (4.38 mL, 25.1 mmol). After stirring at RT for 2 h, the solution was concentrated to dryness at reduced pressure and the residue subjected to flash chromatography (silica gel, 1:1 EtOAc/petroleum ether) to give the desired product (2.5 g, 44%) as a yellow oil. LCMS (ESI) m/z calcd for C₂₅H₄₄N₂O₅: 452. Found: 453 (M+1)⁺. ¹H NMR (400 MHz, CDCl₃) δ 8.14 (d, J=5.6 Hz, 1H), 7.43 (t, J=1.6 Hz, 1H), 7.08 (d, J=2.0 Hz, 1H), 5.39-5.34 (m, 1H), 4.45 (dd, J=12.4, 4.0 Hz, 1H), 4.32 (dd, J=12.4, 6.8 Hz, 1H), 3.75-3.66 (m, 2H), 3.51-3.45 (m, 2H), 2.34-2.28 (m, 2H), 1.60-1.53 (m, 4H), 1.31-1.24 (m, 22H), 0.89-0.86 (m, 6H).

Step D: 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)-3-(nonyloxy)propyl nonanoate. 1-(nonanoyloxy)-3-(nonyloxy)propan-2-yl 1H-imidazole-1-carboxylate (2.50 g, 5.52 mmol) was dissolved in DMF (10 mL) and the solution treated with (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (1.90 g, 3.31 mmol) and Cs₂CO₃ (3.60 g, 11.1 mmol) and the resulting mixture was stirred at RT. After 1 h the reaction mixture was diluted with water (80 mL) and extracted with EtOAc (3×80 mL). The combined EtOAc extracts were dried over Na₂SO₄ and concentrated under vacuum. The residue was subjected to flash chromatography (silica gel, 10:1 DCM/MeOH) to give the desired product (1.6 g, 30%) as a colorless oil. LCMS (ESI) m/z calcd for C₅₂H₇₂FN₅O₉Si: 957. Found: 958 (M+1)⁺. ¹H NMR (400 MHz, CDCl₃) δ 8.21-7.99 (m, 2H), 7.67-7.64 (m, 4H), 7.46-7.36 (m, 6H), 6.53 (t, J=6.8 Hz, 1H), 5.81 (dd, J=6.8, 3.6 Hz, 1H), 5.34-5.18 (m, 1H), 4.41 (dd, J=12.0, 4.0 Hz, 1H), 4.29 (dd, J=12.0, 6.8 Hz, 1H), 4.05 (d, J=11.2 Hz, 1H), 3.96 (d, J=11.2 Hz, 1H), 3.69-3.60 (m, 2H), 3.51-3.43 (m, 2H), 2.89-2.82 (m, 1H), 2.74-2.64 (m, 1H), 2.59 (s, 1H), 2.33 (t, J=7.6 Hz, 2H), 2.16 (s, 3H), 1.64-1.56 (m, 4H), 1.32-1.24 (m, 22H), 1.09 (s, 9H), 0.90-0.84 (m, 6H).

Step E: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)-3-(nonyloxy)propyl nonanoate—diastereomers 1 and 2. To a solution of 2-(((9-((2R,4S,5R)-4-acetoxy-5-(((tert-butyldiphenylsilyl)oxy)methyl)-5-ethynyltetrahydrofuran-2-yl)-2-fluoro-9H-purin-6-yl)carbamoyl)oxy)-3-(nonyloxy)propyl nonanoate (1.6 g, 1.670 mmol) in THF (10 mL) was added 1M TBAF/THF (3.34 mL, 3.34 mmol), and the resulting solution was stirred at RT. After 16 h, the reaction mixture was diluted with water (30 mL) and extracted with EtOAc (3×30 mL). The combined organic phases were dried over Na₂SO₄ and concentrated under vacuum. The residue was subjected to flash chromatography (silica gel, 2:1 petroleum ether/EtOAc) to give 2-(((9-((2R,4S,5R)-4-acetoxy-5-ethynyl-5-(hydroxymethyl)tetrahydrofuran-2-yl)-2-fluoro-9H-purin-6-yl)carbamoyl)oxy)-3-(nonyloxy)propyl nonanoate as a mixture of C-2 diastereomers as a colorless oil. The diastereomers were separated by preparative HPLC (CHIRALPAK IE, 3×25 cm column, 5 um; Mobile Phase A: MTBE containing 10 mM NH₃/MeOH, Mobile Phase B: EtOH; Flow rate: 35 mL/min; isocratic at 15% B over 12 minutes). Then the diastereomers were independently subjected to NaOMe saponification as described herein, followed by RP-HPLC purification (C18, MeCN/10 mM NH₄HCO₃+0.1% NH4OH) to afford the title compounds as white solids, both in 9% yield. The C-2 absolute configurations were not determined. Data for diastereomer 1: LCMS (ESI) m/z calcd for C₃₄H₅₂FN₅O₈: 677. Found: 678 (M+1)⁺. ¹H NMR (400 MHz, CDCl₃) δ 8.41 (s, 1H), 8.00 (s, 1H), 6.40 (dd, J=8.8, 5.6 Hz, 1H), 5.26 (dd, J=6.4, 3.6 Hz, 1H), 4.73 (d, J=5.6 Hz, 1H), 4.38-4.27 (m, 2H), 4.07 (d, J=12.4 Hz, 1H), 3.88 (d, J=12.4 Hz, 1H), 3.66 (d, J=4.8 Hz, 2H), 3.52-3.40 (m, 2H), 3.13-3.06 (m, 1H), 2.82 (s, 1H), 2.55-2.49 (m, 1H), 2.32 (t, J=7.6 Hz, 2H), 1.62-1.52 (m, 4H), 1.47-1.24 (m, 22H), 0.88-0.84 (m, 6H). Data for diastereomer 2: LCMS (ESI) m/z calcd for C₃₄H₅₂FN₅O₈: 677. Found: 678 (M+1)⁺. ¹H NMR (400 MHz, CDCl₃) δ 8.36 (s, 1H), 8.02 (s, 1H), 6.40 (dd, J=8.8, 5.6 Hz, 1H), 5.26 (dd, J=6.8, 4.0 Hz, 1H), 4.73 (d, J=5.4 Hz, 1H), 4.40-4.31 (m, 2H), 4.07 (d, J=12.6 Hz, 1H), 3.88 (d, J=12.6 Hz, 1H), 3.66 (d, J=4.8 Hz, 2H), 3.53-3.39 (m, 2H), 3.12-3.07 (m, 1H), 2.82 (s, 1H), 2.55-2.49 (m, 1H), 2.32 (t, J=7.6 Hz, 2H), 1.62-1.52 (m, 4H), 1.47-1.24 (m, 22H), 0.88-0.84 (m, 6H).

### Examples 37 and 38: 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)-3-(pentyloxy)propyl pentanoate—diastereomers 1 and 2

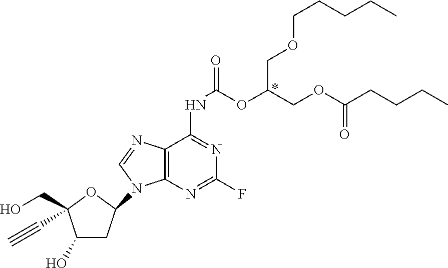 [SMILES: C#C[C@]1(CO)O[C@@H](n2cnc3c(NC(=O)OC(COCCCCC)COC(=O)CCCC)nc(F)nc32)C[C@@H]1O]

The title compounds were prepared as described herein for the synthesis of 2-(((9-((2R,4S,5R)-5-ethynyl-4-hydroxy-5-(hydroxymethyl)tetrahydrofuran-2-yl)-2-fluoro-9H-purin-6-yl)carbamoyl)oxy)-3-(nonyloxy)propyl nonanoate, substituting pentan-1-ol for nonan-1-ol in step A. The C-2 absolute configurations were not determined. Data for diastereomer 1: LCMS (ESI) m/z calcd for C₂₆H₃₆FN₅O₈: 565. Found: 566 (M+1)⁺. ¹H NMR (400 MHz, CDCl₃) δ 8.68 (s, 1H), 8.04 (s, 1H), 6.40 (dd, J=8.0, 6.0 Hz, 1H), 5.28-5.25 (m, 1H), 5.00 (br, s, 1H), 4.75 (s, 1H), 4.41-4.28 (m, 2H), 4.08 (d, J=12.4 Hz, 1H), 3.89 (d, J=12.4 Hz, 1H), 3.68 (dd, J=5.2, 0.8 Hz, 2H), 3.54-3.43 (m, 2H), 3.12-3.08 (m, 1H), 2.81 (s, 1H), 2.66 (br, s, 1H), 2.57-2.51 (m, 1H), 2.32 (t, J=7.6 Hz, 2H), 1.62-1.54 (m, 4H), 1.38-1.28 (m, 6H), 0.90-0.86 (m, 6H). Data for diastereomer 2: LCMS (ESI) m/z calcd for C₂₆H₃₆FN₅O₈: 565. Found: 566 (M+1)⁺. ¹H NMR (400 MHz, CDCl₃) δ 8.68 (s, 1H), 8.04 (s, 1H), 6.41 (d, J=6.0 Hz, 1H), 5.27 (s, 1H), 4.73 (s, 1H), 4.42-4.28 (m, 2H), 4.08 (d, J=12.4 Hz, 1H), 3.89 (d, J=12.4 Hz, 1H), 3.67 (d, J=8.0 Hz, 2H), 3.50-3.43 (m, 2H), 3.09 (br, s, 1H), 2.81 (s, 1H), 2.66 (br, s, 1H), 2.54-2.51 (m, 1H), 2.33 (t, J=7.6 Hz, 2H), 1.64-1.56 (m, 4H), 1.38-1.30 (m, 6H), 0.90-0.87 (m, 6H).

### Anti-HIV Activity

### PSV Assay

A pseudotyped virus assay (PSV) was used to assess the potency of the HIV inhibitor. Replication defective virus was produced by co-transfection of a plasmid containing an NL4-3 provirus [containing a mutation in the envelope open reading frame (ORF) and a luciferase reporter gene replacing the nef ORF] and a CMV-promoter expression plasmid containing an ORF for various HIV gp160 envelope clones. The harvested virus was stored at −80 C in small aliquots and the titer of the virus measured to produce a robust signal for antiviral assays.

The PSV assay was performed by using U373 cells stably transformed to express human CD4, the primary receptor for HIV entry and either human CXCR4 or human CCR5 which are the co-receptors required for HIV entry as target cells for infection. Molecules of interest (including, but not limited to small molecule inhibitors of HIV, neutralizing antibodies of HIV, antibody-drug conjugate inhibitors of HIV, peptide inhibitors of HIV, and various controls) are capable of being diluted into tissue culture media and diluted via serial dilution to create a dose range of concentrations, and this was carried out for Example 1. This dose-range was applied to U373 cells and the pre-made pseudotyped virus added. The amount of luciferase signal produced after 3 days of culture was used to reflect the level of pseudotyped virus infection. An IC₅₀, or the concentration of inhibitor required to reduce PSV infection by 50% from the infection containing no inhibitor was calculated. Assays to measure cytotoxity were performed in parallel to ensure the antiviral activity observed for an inhibitor was distinguishable from reduced target cell viability. IC₅₀ values were determined from a 10 point dose response curve using 3-4-fold serial dilution for each compound, which spans a concentration range >1000 fold.

These values are plotted against the molar compound concentrations using the standard four parameter logistic equation:

y=((Vmax*x\hat{ }n)/(K\hat{ }n+x\hat{ }n))+Y2

where:

Y2=minimum y n=slope factor

Vmax=maximum y x=compound concentration [M]

K=EC₅₀

The resulting data is shown in Table 3.

TABLE 3

| Example | WT IC₅₀ (μM) | M184V IC₅₀ (μM) |
|---|---|---|
| EFdA | 0.0065 | 0.0351 |
| 1 | >25 | >25 |
| 2 | >25 | >25 |
| 3 | 2.0140 | 8.1730 |
| 4 | 2.6610 | >25 |
| 5 | 15.4200 | >25 |
| 6 | 0.5605 | 1.0800 |
| 7 | 1.2840 | 2.4120 |
| 8 | 0.1419 | 1.1300 |
| 9 | 0.0049 | 0.0221 |
| 10 | 0.0097 | 0.0313 |
| 11 | 2.3110 | 3.2110 |
| 12 | 0.0170 | 0.0663 |
| 13 | 0.0072 | 0.0178 |
| 14 | 1.5540 | 2.4080 |
| 15 | >15 | >15 |
| 16 | 15.0000 | 22.4000 |
| 17 | >15 | >15 |
| 18 | 2.0320 | 9.1080 |
| 19 | 1.7690 | 9.4390 |
| 20 | 10.5400 | >25 |
| 21 | 14.7200 | >25 |
| 22 | 11.6900 | >25 |
| 23 | >25 | >25 |
| 24 | 1.4800 | 3.7360 |
| 25 | 1.1830 | 1.2580 |
| 26 | 16.1200 | 17.3200 |
| 27 | >25 | >25 |
| 28 | 0.0106 | 0.0392 |
| 29 | 0.0077 | 0.0283 |
| 30 | 0.0077 | 0.0293 |
| 31 | 0.0151 | 0.0478 |
| 32 | 0.0075 | 0.0269 |
| 33 | 0.0054 | 0.0243 |
| 34 | 0.0132 | 0.0678 |
| 35 | 0.0112 | 0.0331 |
| 36 | 0.0085 | 0.0229 |
| 37 | 0.0095 | 0.0324 |
| 38 | 0.0070 | 0.0234 |

### Rat Pharmacokinetics Data for Compounds 9 and 30 (Examples 9 and 30)

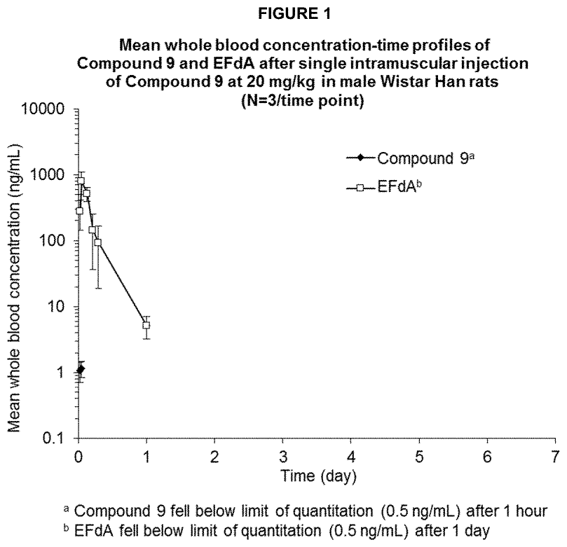

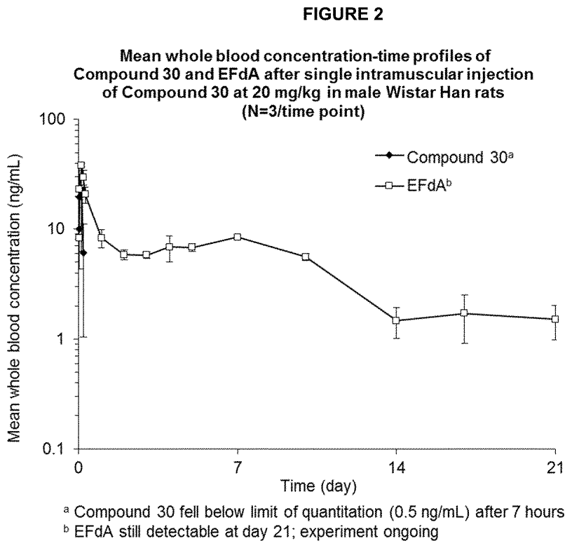

The pharmacokinetics of Compounds 9 and 30 after a single intramuscular (IM) injection were evaluated in Male Wistar Han rats. The test compound was suspended in a 2% P407, 2% PEG3350, 3.5% Mannitol formulation at 10 mg/mL concentration. A single dose of 20 mg/kg of test compound was injected (2 mL/kg) IM into the right gastrocnemius muscle (n=3). Blood samples were collected via a lateral tail vein at the following time points: Day 1 [30 min, 1 h, 3 h, 5 h, 7 h], Days 2-5, Days 7, 10, 14, 17, 21, 24, 28, 31, 35, 38, 42, 45, 49, 52, 56, 59, 63, 66 and 70, etc. To evaluate test compound and EFdA concentrations, approximately 150 μL of blood was collected into a NaFL/Na2EDTA tube. Exactly 150 μL of blood was then pipetted into a new tube, mixed with 150 μL of 100 mM ammonium acetate pH4, (some of the prodrugs required mixing with 1.5 μL of FA as stabilizer), vortexed, immediately frozen on dry ice and stored at −80° C. until analysis. For analysis of test compound and EFdA, frozen blood samples were thawed and mixed with 200 μL of internal standard solution (20 ng/mL Glipizide in acetonitrile), vortexed for 10 min at 750 rpm and centrifuged at 6000 rpm for 10 min. The supernatants were then analyzed by UPLC/MS-MS (Triple Quad™ 6500+). Pharmacokinetic parameters were evaluated using a non-compartmental model of the non-compartmental analysis tool, Pharsight Phoenix WinNonlin® 6.4 software. Results are provided in FIGS. 1 and 2.

1. A compound of the formula (I): 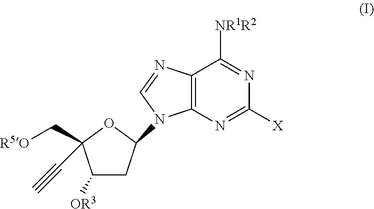
wherein:
X is selected from the group consisting of NH₂, F and Cl;
R¹ and R² are each independently selected from the group consisting of H, 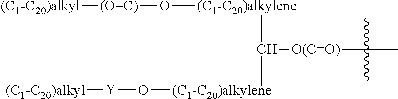
wherein Y is a bond or —C(═O)—; and 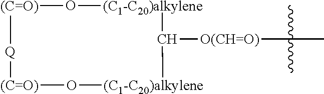
wherein Q is (C₃-C₁₅)alkylene;
R³ is selected from the group consisting of H and —C(═O)—Z—R⁴, wherein Z is a bond or —O—, and wherein R⁴ is selected from the group consisting of:
(1) NR⁵R⁶ wherein R⁵ is H or (C₁-C₆)alkyl and R⁶ is selected from the group consisting of (C₁-C₂₀)alkyl, (C₃-C₆)cycloalkyl; —(CH₂)ₘ—(C═O)—O—(C₁-C₂₀)alkyl wherein m is an integer ranging from 1 to 10; or wherein R⁵ and R⁶ join to form a (C₃-C₆)heterocycle; 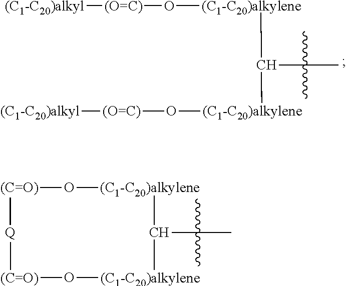
wherein Q is (C₃-C₁₅)alkylene; and
(4) —(C₁-C₆)alkyl-aryl-R¹¹, wherein R¹¹ is selected from H and —O(C═O)—(C₁-C₂₀)alkyl
and R^{5′} is selected from the group consisting of H, 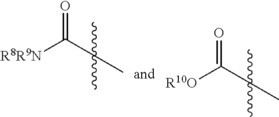
wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₀)alkyl; 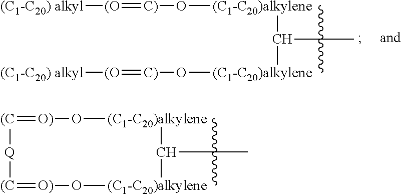
wherein Q is (C₃-C₁₅)alkylene;
wherein when R³ and R^{5′} are each H and X is F or Cl, both R¹ and R² cannot be H;
or a pharmaceutically acceptable salt thereof.

2. The compound according to claim 1, wherein X is F.

3. The compound according to claim 1, wherein R¹ and R² are each H.

4. The compound according to claim 1, wherein R¹ is H and R² is selected from 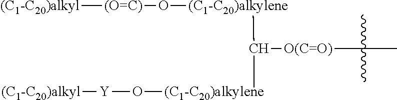
wherein Y is a bond or —C(═O)—
; and 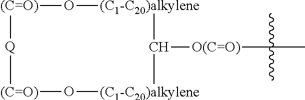
wherein Q is (C₃-C₁₅)alkylene.

5. The compound according to claim 1, wherein R³ is H.

6. The compound according to claim 1, wherein R³ is C(═O)—Z—R⁴,

7. The compound according to claim 6, wherein Z is a bond and R⁴ is NR⁵R⁶, R⁵ is H or (C₁-C₆)alkyl and R⁶ is selected from the group consisting of (C₁-C₂₀)alkyl, (C₃-C₆)cycloalkyl; —(CH₂)ₘ—(C═O)—O—(C₁-C₂₀)alkyl wherein m is an integer ranging from 1 to 10; or wherein R⁵ and R⁶ join to form a (C₃-C₆)heterocycle.

8. The compound according to claim 6, wherein Z is —O— and R⁴ is:

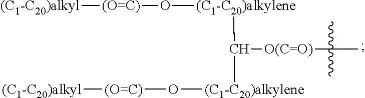

9. The compound according to claim 1, wherein R^{5′} is H.

10. The compound according to claim 1, wherein R^{5′} is: 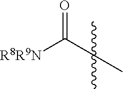
wherein R⁸ is H or (C₁-C₆)alkyl and R⁹ is (C₁-C₂₀)alkyl.

11. The compound according to claim 1, wherein R^{5′} is: 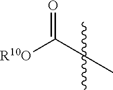
wherein R¹⁰ is selected from —(C₁-C₆)alkyl-aryl-R¹¹, wherein R¹¹ is selected from H and —O(C═O)—(C₁-C₂₀)alkyl; 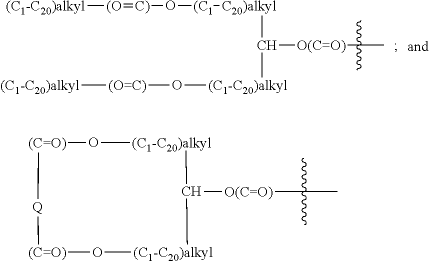
wherein Q is (C₃-C₁₅)alkyl;

12. A compound of the formula (II): 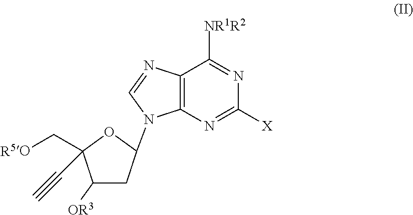
wherein:
X is selected from the group consisting of NH₂, F and Cl;
R¹ and R² are each independently selected from the group consisting of H, 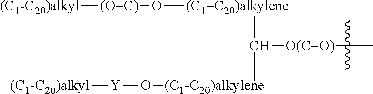
wherein Y is a bond or —C(═O)—
and 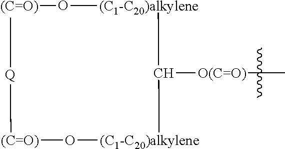
wherein Q is (C₃-C₁₅)alkylene;
R³ is selected from the group consisting of H and —C(═O)—Z—R⁴, wherein Z is a bond or —O—, and wherein R⁴ is selected from the group consisting of:
(1) NR⁵R⁶ wherein R⁵ is H or (C₁-C₆)alkyl and R⁶ is selected from the group consisting of (C₁-C₂₀)alkyl, (C₃-C₆)cycloalkyl; —(CH₂)ₘ—(C═O)—O—(C₁-C₂₀)alkyl wherein m is an integer ranging from 1 to 10; or wherein R⁵ and R⁶ join to form a (C₃-C₆)heterocycle; 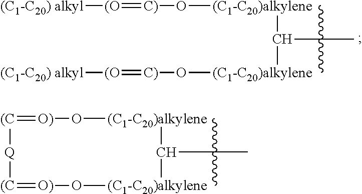
wherein Q is (C₃-C₁₅)alkylene; and
(4) —(C₁-C₆)alkyl-aryl-R¹¹, wherein R¹¹ is selected from H and —O(C═O)—(C₁-C₂₀)alkyl
and R^{5′} is selected from the group consisting of H, 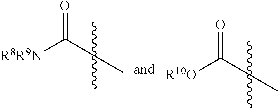
wherein R⁸ is H or (C₁-C₆)alkyl; R⁹ is (C₁-C₂₀)alkyl, and R¹⁰ is selected from —(C₁-C₆)alkylene-aryl-R¹¹, wherein R¹¹ is selected from H and —O(C═O)—(C₁-C₂₀)alkyl; 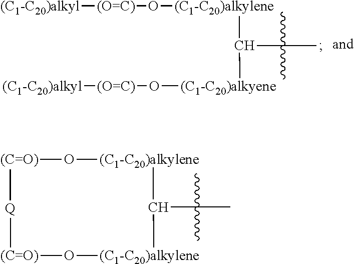
wherein Q is (C₃-C₁₅)alkylene;
wherein when R³ and R⁵ are each H and X is F or Cl, both R¹ and R² cannot be H;
or a pharmaceutically acceptable salt thereof.

13. A compound selected from the group consisting of: 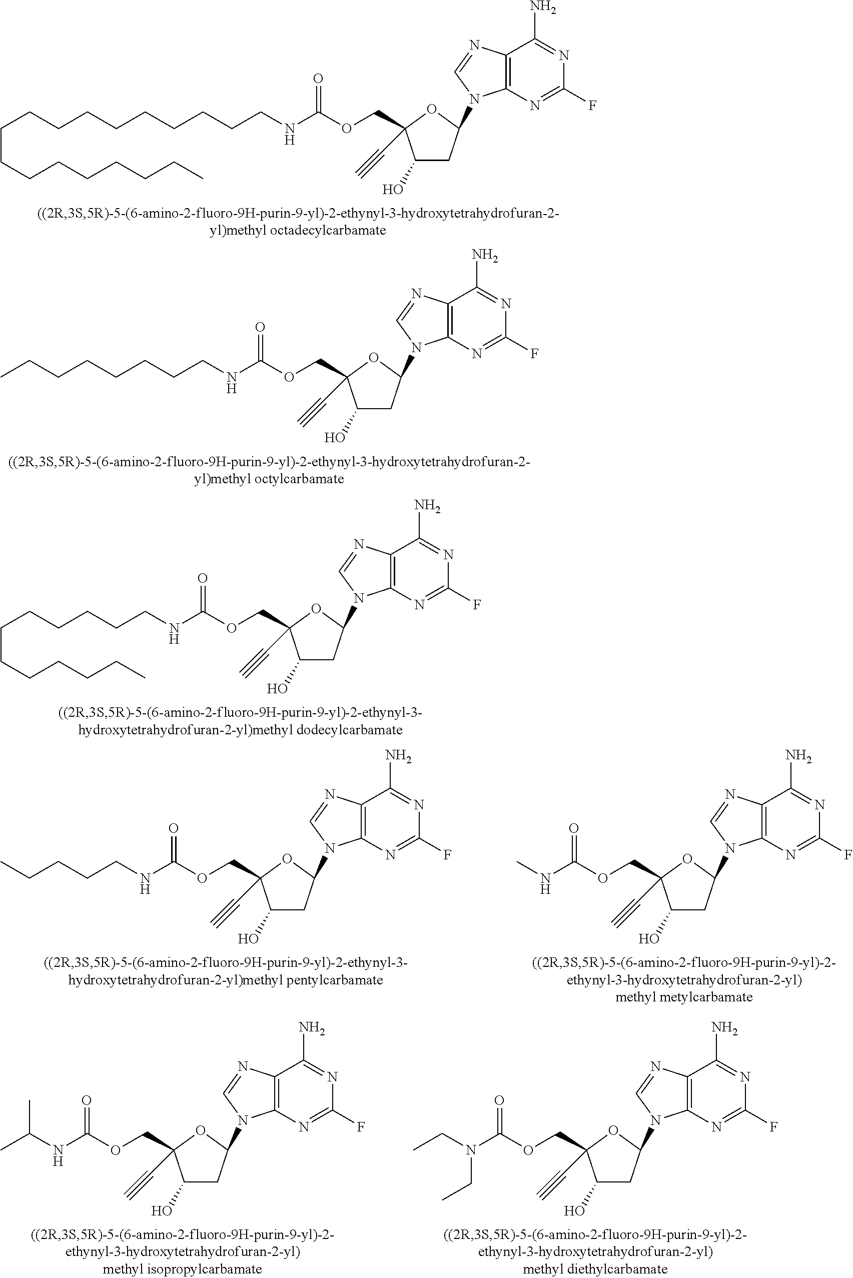 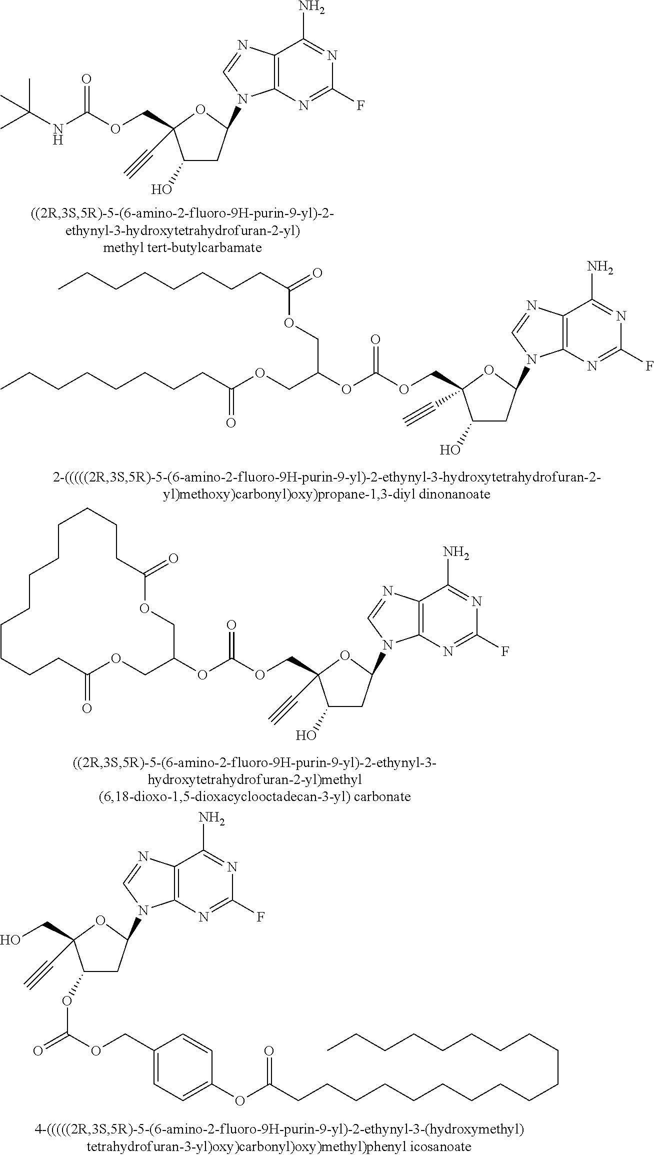 [SMILES: C#C[C@]1(COC(=O)NC(C)(C)C)O[C@@H](n2cnc3c(N)nc(F)nc32)C[C@@H]1O ; C#C[C@]1(COC(=O)OC(COC(=O)CCCCCCCC)COC(=O)CCCCCCCC)O[C@@H](n2cnc3c(N)nc(F)nc32)C[C@@H]1O ; C#C[C@]1(COC(=O)OC2COC(=O)CCCCCCCCCCCC(=O)OC2)O[C@@H](n2cnc3c(N)nc(F)nc32)C[C@@H]1O ; C#C[C@]1(CO)O[C@@H](n2cnc3c(N)nc(F)nc32)C[C@@H]1OC(=O)OCc1ccc(OC(=O)CCCCCCCCCCCCCCCCCCC)cc1] 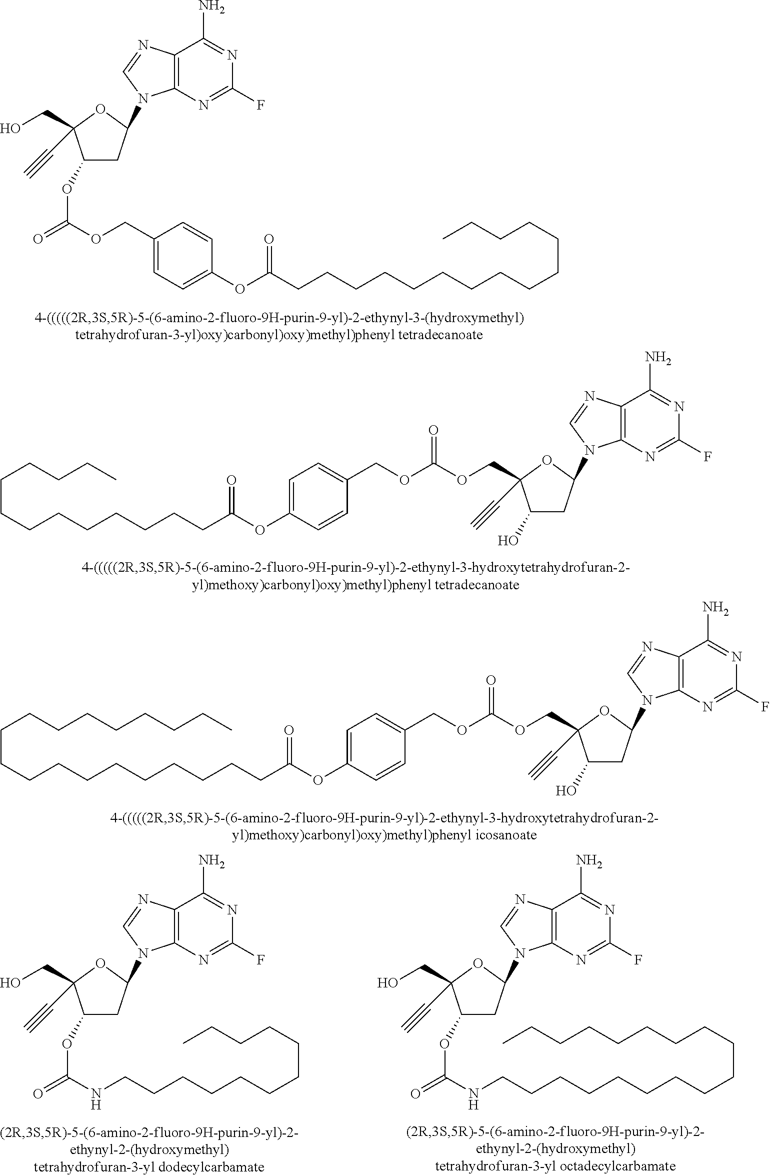 [SMILES: C#C[C@]1(CO)O[C@@H](n2cnc3c(N)nc(F)nc32)C[C@@H]1OC(=O)OCc1ccc(OC(=O)CCCCCCCCCCCCCCC)cc1 ; C#C[C@]1(COC(=O)OCc2ccc(OC(=O)CCCCCCCCCCCCC)cc2)O[C@@H](n2cnc3c(N)nc(F)nc32)C[C@@H]1O ; C#C[C@]1(COC(=O)OCc2ccc(OC(=O)CCCCCCCCCCCCCCCCCCC)cc2)O[C@@H](n2cnc3c(N)nc(F)nc32)C[C@@H]1O ; C#C[C@]1(CO)O[C@@H](n2cnc3c(N)nc(F)nc32)C[C@@H]1OC(=O)NCCCCCCCCCCCC ; C#C[C@]1(CO)O[C@@H](n2cnc3c(N)nc(F)nc32)C[C@@H]1OC(=O)NCCCCCCCCCCCCCCCCCC] 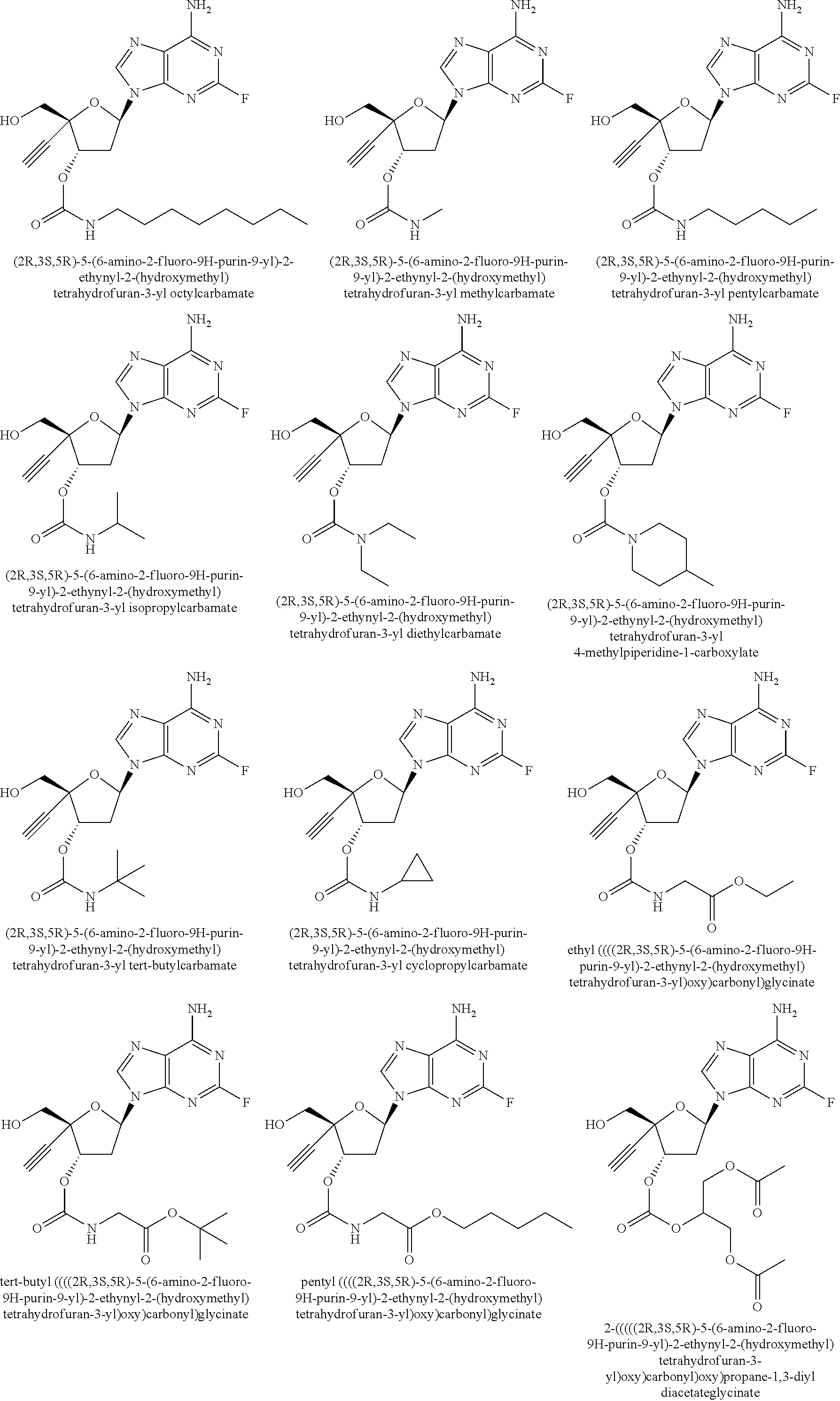 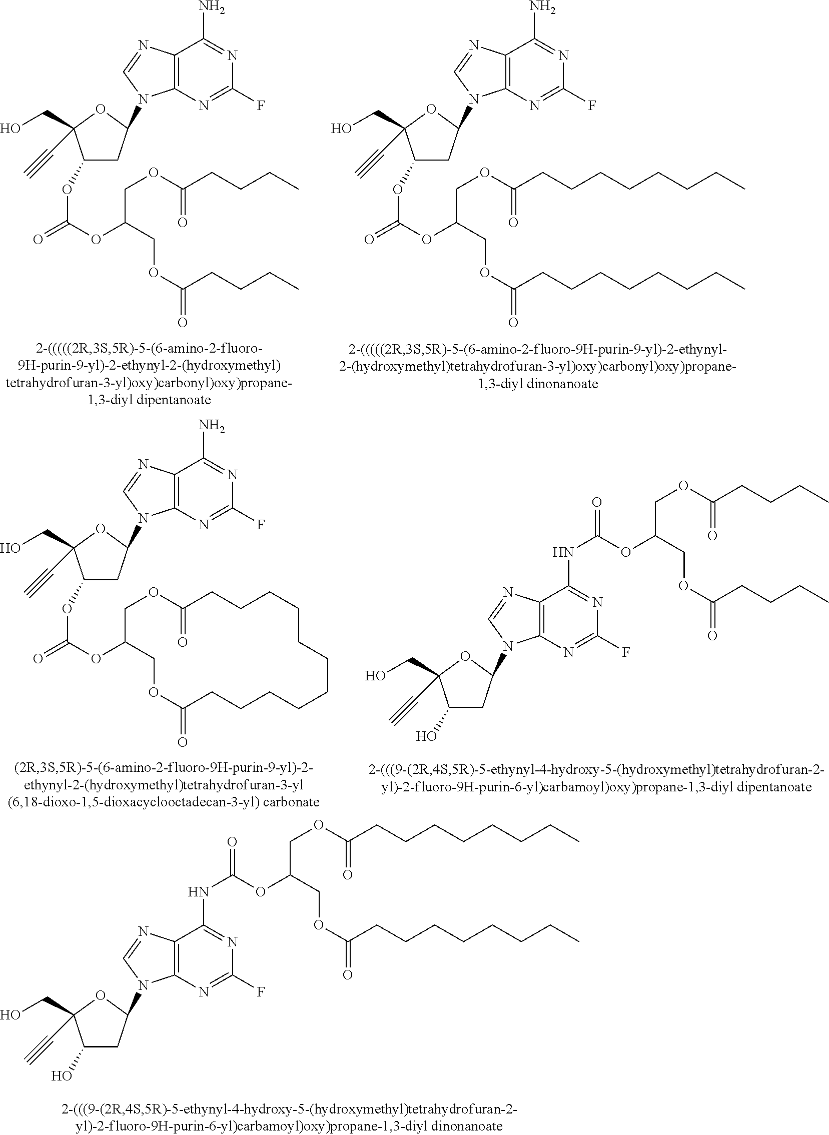 [SMILES: C#C[C@]1(CO)O[C@@H](n2cnc3c(N)nc(F)nc32)C[C@@H]1OC(=O)OC(COC(=O)CCCC)COC(=O)CCCC ; C#C[C@]1(CO)O[C@@H](n2cnc3c(N)nc(F)nc32)C[C@@H]1OC(=O)OC(COC(=O)CCCCCCCC)COC(=O)CCCCCCCC ; C#C[C@]1(CO)O[C@@H](n2cnc3c(N)nc(F)nc32)C[C@@H]1OC(=O)OC1COC(=O)CCCCCCCCCCCC(=O)OC1 ; C#C[C@]1(CO)O[C@@H](n2cnc3c(NC(=O)OC(COC(=O)CCCC)COC(=O)CCCC)nc(F)nc32)C[C@@H]1O ; C#C[C@]1(CO)O[C@@H](n2cnc3c(NC(=O)OC(COC(=O)CCCCCCCC)COC(=O)CCCCCCCC)nc(F)nc32)C[C@@H]1O] 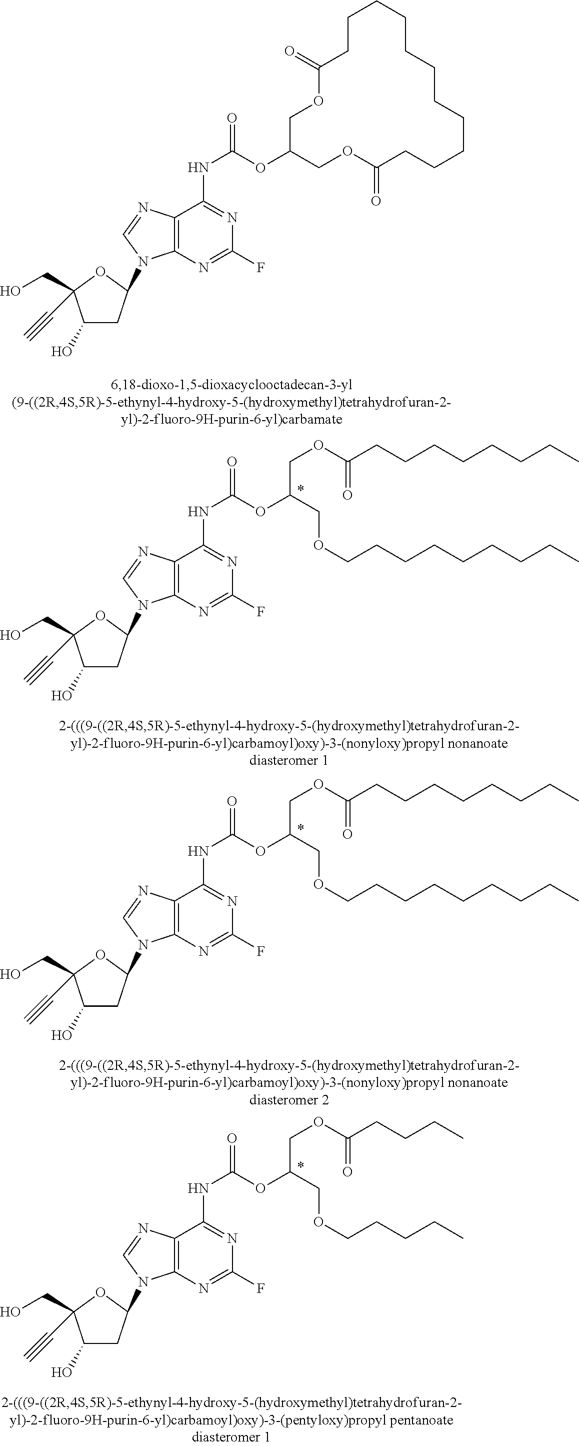 [SMILES: C#C[C@]1(CO)O[C@@H](n2cnc3c(NC(=O)OC4COC(=O)CCCCCCCCCCCC(=O)OC4)nc(F)nc32)C[C@@H]1O ; C#C[C@]1(CO)O[C@@H](n2cnc3c(NC(=O)OC(COCCCCCCCCC)COC(=O)CCCCCCCC)nc(F)nc32)C[C@@H]1O ; C#C[C@]1(CO)O[C@@H](n2cnc3c(NC(=O)OC(COCCCCCCCCC)COC(=O)CCCCCCCC)nc(F)nc32)C[C@@H]1O ; C#C[C@]1(CO)O[C@@H](n2cnc3c(NC(=O)OC(COCCCCC)COC(=O)CCCC)nc(F)nc32)C[C@@H]1O] 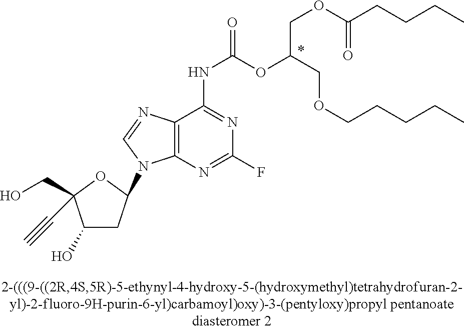 [SMILES: C#C[C@]1(CO)O[C@@H](n2cnc3c(NC(=O)OC(COCCCCC)COC(=O)CCCC)nc(F)nc32)C[C@@H]1O]
and pharmaceutically acceptable salts thereof.

14. A pharmaceutical composition comprising a compound according to claim 1, or a pharmaceutically acceptable salt thereof, and a pharmaceutically acceptable excipient.

15. The composition of claim 14, wherein the composition is present in parenteral form.

16. The composition of claim 14, wherein the composition is in a tablet form.

17. A method of treating an HIV infection in a subject comprising administering to the subject a compound of claim 1, or a pharmaceutically acceptable salt thereof.

18. (canceled)

19. A method of preventing an HIV infection in a subject at risk for developing an HIV infection, comprising administering to the subject a compound of claim 1, or a pharmaceutically acceptable salt thereof.

20-24. (canceled)
